(19) United States
(12) Patent Application Publication
(10) Pub. No.: US 2016/0054307 A1
(43) Pub. Date: Feb. 25, 2016

(54) LED ASSAY READER WITH TOUCHSCREEN CONTROL AND BARCODE SAMPLE ID

(71) Applicant: Immunetics, Inc., Boston, MA (US)

(72) Inventors: Richard A. Pinkowitz, Newton, MA (US); Michael J. Ockham, Tewksbury, MA (US); Gregory V. Williams, Jamaica Plains, MA (US); Neil X. Krueger, Roslindale, MA (US); Andrew Han, Cambridge, MA (US); Lynda S. Mungo, Attleboro, MA (US); James L. Dowling, New Boston, NH (US); Jason S. Minahan, Billerica, MA (US); Jack Kessler, Southborough, MA (US); Gary Chevalier, Merrimack, NH (US); Roger T. Lacroix, Bedford, NH (US)

(21) Appl. No.: 14/665,574

(22) Filed: Mar. 23, 2015

Related U.S. Application Data
(62) Division of application No. 14/513,991, filed on Oct. 14, 2014.
(60) Provisional application No. 61/889,874, filed on Oct. 11, 2013.

(51) Int. Cl. G01N 33/50; G01N 21/27

(52) U.S. Cl. CPC G01N 33/5091; G01N 21/274; G01N 21/272; G01N 2333/90245

(57) ABSTRACT

Assay devices, assay detection systems, and methods comprising same for analytical tests, medical assays, diagnostic tests, medical diagnosis, risk assessment, or quality control purposes are provided. These devices, systems, and methods are designed to be employed at the point of care, such as in emergency rooms, operating rooms, hospital laboratories and other clinical laboratories, doctor's offices, in the field, or in any situation in which a rapid and accurate result is desired. The systems and methods process samples, such as clinical, biological, or blood sample, and read data from colorimetric based biochemical assays to provide an indication of the presence or absence of a bacterial, fungal, or viral contaminants therein. The assay devices include an optical reader apparatus and barcode scanner for reading and matching the test results to identification information provided by the barcodes to facilitate ease of tracking compliant and noncompliant samples.

### RELATED APPLICATION

This application is a divisional application of U.S. application Ser. No. 14/513,991, filed Oct. 14, 2014; which claims the benefit of U.S. Provisional Application No. 61/889,874, filed Oct. 11, 2013; the contents of which is hereby incorporated by reference.

### FIELD OF THE INVENTION

This invention relates to an optical reader apparatus, software, assay device, and assay detection systems and methods comprising same for rapid, high throughput, and easy detection of contaminants in a sample. The assay devices and detection systems are particularly useful for providing point-of-care testing for a variety of medical applications and quality control tests. Such assay detection systems comprise an optical reader apparatus and software utilizing colorimetric assays and methods for detecting the presence of a bacterial, fungal pathogen, or contaminants in a biological sample (for example, fluid, blood, urine, saliva, etc.).

### BACKGROUND OF THE INVENTION

On Mar. 1, 2004, American Association of Blood Banks (AABB) standards mandated that United States blood centers commence testing all platelet units for bacterial contamination. This new standard was based on the significant risks to transfusion patients associated with contaminated platelet units. (Standards for blood banks and transfusion services. Bethesda: American Association of Blood Banks; 2004.) Further safeguards were made effective in January 2011 with the implementation of AABB Interim directive 5.1.5.1.1, which specified that such bacterial testing methods must have prior FDA clearance or must demonstrate equivalent sensitivity to FDA-cleared methods (Interim standard 5.1.5.1.1. Association bulletin 10-02 (May 3, 2010). Bethesda, Md.: AABB, 2010.)

Bacteria are the most common contaminating infectious agents found in platelet units. It has been estimated that the rate of bacterial contamination for apheresis platelet donations is 1 in 5000, making platelets the most frequent source of transfusion related infection (Eder A F et al., “Bacterial screening of apheresis platelets and the residual risk of septic transfusion reactions: the American Red Cross experience (2004-2006)” Transfusion (2007) 47:1134-1142). Since platelets must be stored at 20-24° C. in order to maintain function, small numbers of bacteria that are mostly introduced from a donor's skin flora into platelet units can multiply to very high numbers in a matter of days (Brecher M E et al., “Growth of bacteria in inoculated platelets: implications for bacteria detection and the extension of platelet storage” Transfusion (2000) 40:1308-1312) Skin flora are not the only source of bacteria that contaminate platelet units; many strains of Gram-positive and Gram-negative bacteria have been identified in contaminated platelet units, including Staphylococcus, Pseudomonas, Bacillus, and Streptococcus species (Jacobs M R et al. “Relationship between bacterial load, species virulence, and transfusion reaction with transfusion of bacterially contaminated platelets” Clin Infect Dis (2008) 46:1214-1220).

Approximately 4 million platelet units are transfused per year in the U.S., of which up to 4000 are potentially contaminated. Contaminated platelet units have been identified as a cause of sepsis-related morbidity and mortality. Even at early time points in the mandatory maximum five-day storage time limit post-collection, microbial growth may reach significant levels. A quick and easy assay device for detecting bacterial contamination in a sample is needed. Such an assay device could track and match samples that are free of contamination and facilitate a ready supply of biological and clinical products that are free of bacterial and fungal contamination and safe for use by humans.

This innovation is extremely significant due to the great demand in the blood bank industry for a rapid, sensitive, and specific assay device for bacteria in platelet units. Therefore, it would be advantageous to have an assay device that enables the user to detect and measure bacterial, fungal, or pathogenic contamination in biological samples, such as blood, and a means for matching the data to sample identification, patient ID, product ID, or other biometric signatures. It is also desirable to have such assay devices that can provide fast, efficient, and high throughput analyses and increase the efficiency and time of providing safes samples for use in a variety of medical applications.

Therefore, it is an object herein to provide assay devices, software, assay detection systems, and methods using same for assessing bacterial, fungal, or pathogenic contamination in biological samples. Such devices and systems may be adapted for any chemical assays, nucleic acid assays, fluorometric assays, chemiluminescent, bioluminescent assays, or colorimetric assay that are useful for assessing the quality and compliance of a biological sample, or used in conjunction with point-of-care diagnostic assays.

### SUMMARY OF THE INVENTION

The present invention provides an optical reader apparatus, software, and barcode scanner for monitoring and data collection of colorimetric-based biochemical assays. The optical reader apparatus has an integrated mechanical agitation (sample mixing) function coupled to a sample mixing subsystem. The optical reader apparatus is capable of measuring optical density of single or multiple samples simultaneously and independently, being fully random-access and asynchronous in operation. In one embodiment, the optical system of the reader is solid state, with LED illumination emitting visible wavelengths for transmission through a test sample. In some embodiments, solid state detectors are used to monitor and quantify optical density changes in the sample via the level of light transmitted. The optical reader apparatus's optical system is self-calibrating and reads up to 2.0 standard optical density (OD) units, and can discriminate between positive and negative samples in an automated fashion.

The optical reader apparatus is equipped with onboard single-board computer and data storage, a touchscreen monitor for user interaction, and standard USB 2.0 ports for connectivity and data retrieval. In certain embodiments, the ISBT Barcodes (1D and 2D formats) can be used to identify and track test samples, by attaching a barcode scanner to the optical reader apparatus through a USB port. Similarly, USB ports can be used to download data onto a flash drive or similar device. The user interface allows multiple simultaneous user logins with security features to protect assays in process and assay data, including screen locking/timeout and PIN-based login. Color coded labels having unique ID numbers, with corresponding onscreen display of sample-specific color is available for enhanced sample ID and tracking.

The optical reader apparatus is designed in both hardware and software to maximize utility of colorimetric based biochemical assays, such as the BacTX® assay described in U.S. Pat. Nos. 7,598,054 and 8,450,079, or similar colorimetric assays, in screening or clinical laboratory settings, and has ease-of-use features to streamline workflow and reduce the possibility of data entry or sample identity errors. The reader and its software are tailored for specific assays and achieve semi-automated operation relative to use of standard laboratory equipment, and consequently the reader requires much less input and hands-on manipulation from the user following an initial sample preparation step.

The assay device, assay detection system, and methods comprising same enable identification and tracking of samples, such as biological or clinical samples, with specific application to ensuring the safety of platelets for transfusion and for the diagnosis of UTI and bacterial infection of the central nervous system. Such assay device and assay detection system utilize a rapid, sensitive, and specific assay for the detection of bacteria in samples such as platelet units, urine, and cerebrospinal fluid (CSF) samples. The assay is based on the detection of peptidoglycan, a cell wall component of all bacteria. Present in both Gram-negative and Gram-positive bacteria, peptidoglycan can be used to detect bacterial species known as human pathogens and as frequent contaminants of platelet units as well as less common contaminants or slow growing bacterial pathogens. Further, since peptidoglycan is a major structural component of the cell wall it can be easily and rapidly detected in bacterial populations. The assay may also be used to detect β-glucan, a cell wall component of true fungi, such as yeast and molds.

The assay detection system described herein provides real time diagnostic testing that can be done in a rapid time frame so that the resulting test is performed faster than comparable tests that do not employ this system. For example, the exemplified BacTx® assay for eight samples is performed in about 30-45 minutes. In addition, with the devices, methods, and systems provided herein, assays can be performed on site and in the field, such as in a doctor's office, at a bedside, in a stat laboratory, testing facility, operating rooms, hospital laboratories and other clinical laboratories, emergency room or other such locales, particularly where rapid and accurate results are required.

Further features and advantages will be described in the following drawings, detailed description and claims.

### BRIEF DESCRIPTION OF THE DRAWINGS

FIG. 1 is the System Architecture overview.

FIG. 2 is a flow chart of the Single Board Computer (SBC) instrument controller

FIG. 3 is the Interface Board Controller (IBC) Hardware architecture.

FIG. 4 is the Interface Board Controller (IBC) Software architecture.

FIG. 5 is a top perspective view of the mixing subsystem with sample vial holder and reaction tube.

FIG. 6 is a partial bottom view of the mixing subsystem with inserted sample vial holder.

FIG. 7 is a partial top internal view of the mixing subsystem with sample vial holder and reaction tube.

FIG. 8 is a partial top perspective left view of an assay device with cover removed.

FIG. 9 is a partial left side perspective view of an assay device with cover removed.

FIG. 10 is a top perspective view of an assay device.

FIG. 11 is a top perspective left view of an assay device.

FIG. 12 is a partial end perspective right view of an assay device.

FIG. 13 is a top view of an assay device.

FIG. 14 is a left side view of an assay device.

FIG. 15 is a perspective view leftwards into an assay device with cover removed.

FIG. 16 is a perspective view rightwards into an assay device with cover removed.

FIG. 17 is a top perspective view of an assay device with barcode reader. Said barcode reader is connected to the optical reader apparatus through a USB port

FIG. 18 is a top perspective view of an assay reader and bottom perspective view of the assay reader cover.

FIG. 19 is a top perspective view of an assay device showing the graphical user interface.

FIG. 20 shows a top view of a sample vial holder.

FIG. 21 shows a partial right side perspective view downwards to a sample vial holder.

FIG. 22 shows a right view of a sample vial holder.

FIG. 23 shows a front view of a sample vial holder.

FIG. 24 shows a partial right side perspective view of a sample vial holder.

FIG. 25 shows a sample mixing motion for a sample vial holder.

FIGS. 26-29 show in schematic form the basic functions that may be required in an assay device for use in accordance with the invention, as applied to the BacTx® assay.

FIG. 30 depicts a representative Platelet Unit Barcode.

FIG. 31 shows a partial left side perspective view of an assay device.

FIG. 32 shows a histogram of 505 BacTx Specificity Assays (501 negative assays and 4 Initially Reactive assays) in comparison to the assay cutoff.

FIG. 33 depicts the START ASSAY field on the ASSAY GUI page.

FIG. 34 depicts the GUI presenting the user with the following two fields: the BacTx® ID field and the field.

FIG. 35 depicts the reaction tube in the reaction well indicated on the GUI.

FIG. 36 depicts another view of the START ASSAY field on the ASSAY GUI page.

FIG. 37 depicts the GUI presenting the user with the following two fields: the BACTX® ID LABEL field and the field.

FIG. 38 depicts the GUI presenting the user with the following two fields: the PRODUCT INFORMATION CODE field and the BACTX® REAGENT LOT NUMBER field.

FIG. 39 depicts when User removes 300 μl of a processed sample and transfers it into a reagent tube.

FIG. 40 depicts completed steps 1 to 5 in Use Case 0.

FIG. 41 depicts when User touches the field and populates the DIN using the onscreen keyboard.

FIG. 42 depicts when User removes 300 μl of a processed sample and transfers it into a reagent tube.

FIG. 43 depicts when User activates the CONTROL ASSAY field on the ASSAY GUI page.

FIG. 44 depicts when User populates the BACTX® REAGENT LOT NUMBER by scanning the reagent tube lot ID label (this is optional).

FIG. 45 depicts when User activates the STAT ASSAY field on the ASSAY GUI page.

FIG. 46 depicts when GUI requests user to populate the STAT ID field.

FIG. 47 depicts when User can then populate the PRODUCT IDENTIFICATION CODE and the BACTX® REAGENT LOT NUMBER (by scanning) but both of these fields are optional.

FIG. 48 depicts when User navigates to the ACCOUNT LOGIN page by LOGIN button that is present in top bar of several of the GUI screens.

FIG. 49 depicts when User activates the LOGOUT button the top bar in the GUI.

FIG. 50 depicts the GUI displaying the MONITOR page.

FIG. 51 depicts another view of the GUI displaying the MONITOR page.

FIG. 52 depicts when the user inserts a formatted USB FOB into the front USB port of the BacTx® reader.

FIG. 53 depicts when the user selects the assay results of interest.

FIG. 54 depicts the BacTx® Assay Testing Algorithm.

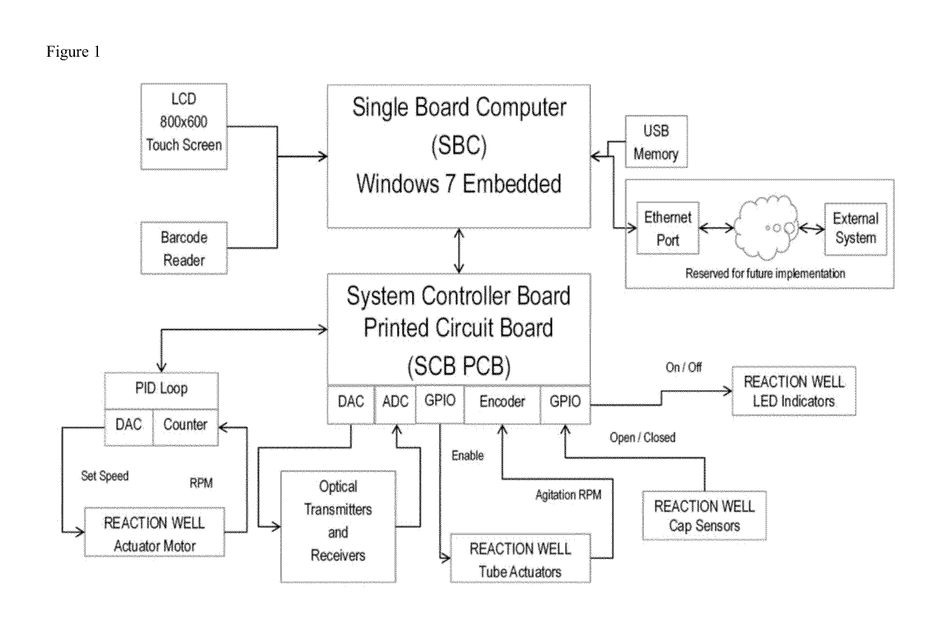

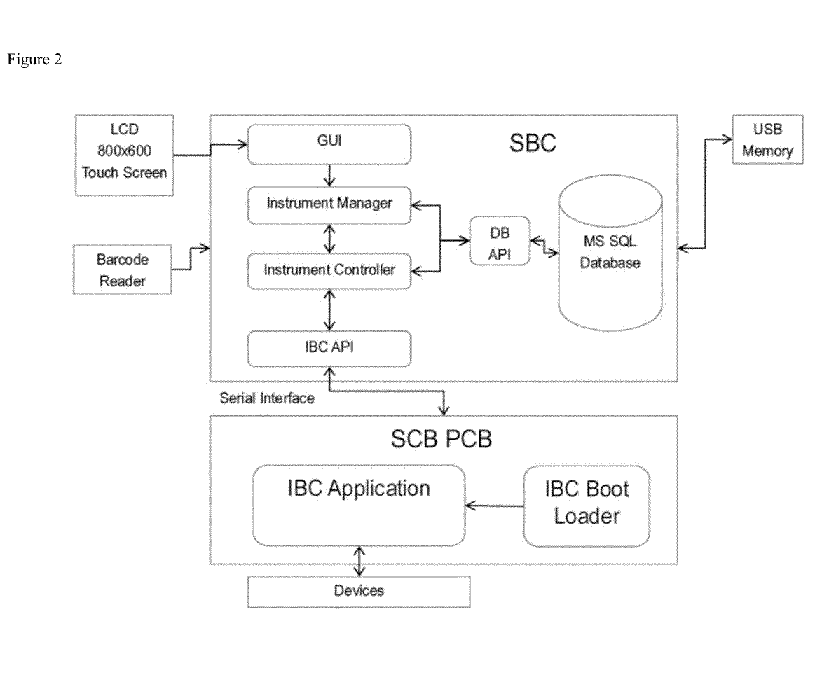

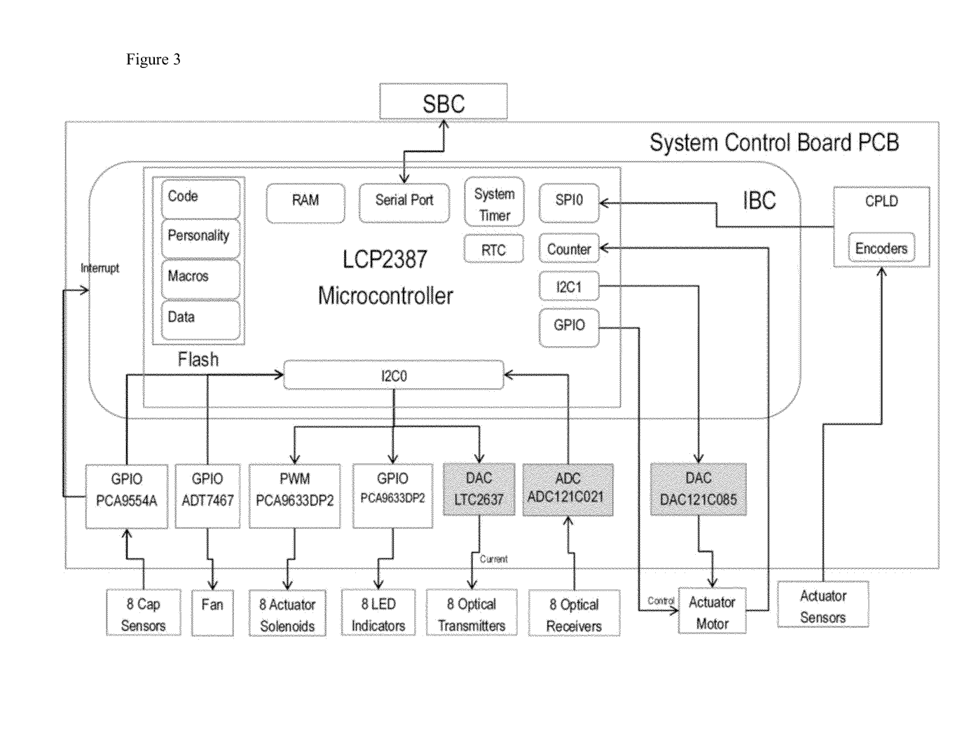

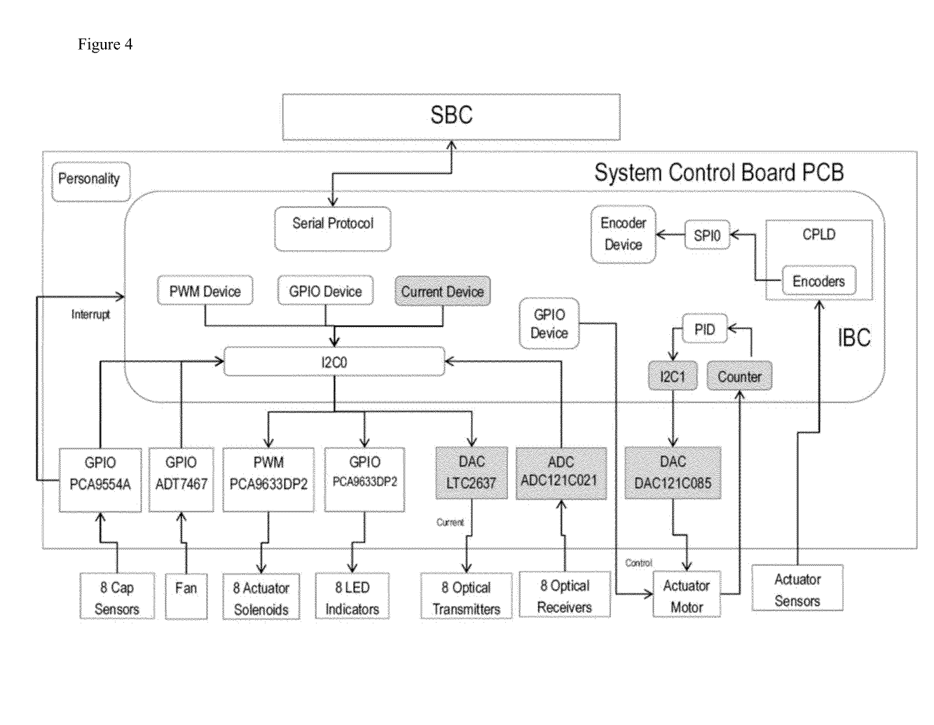

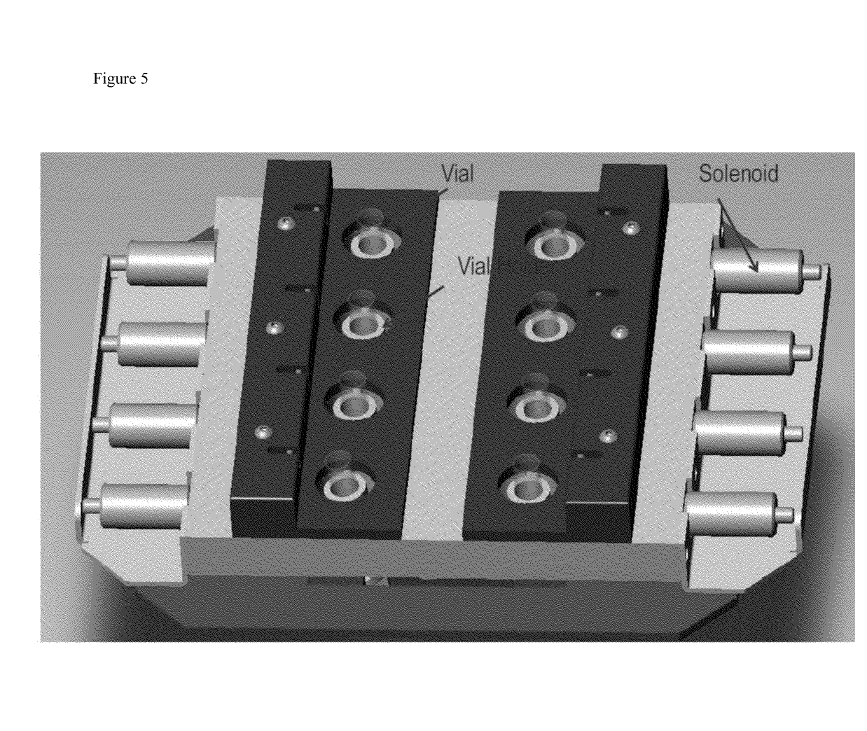

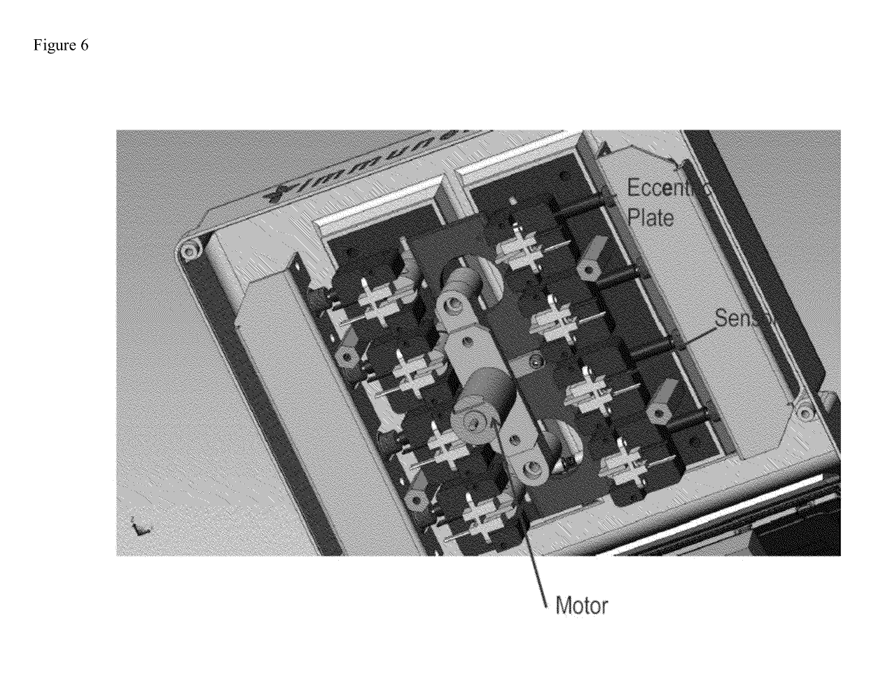

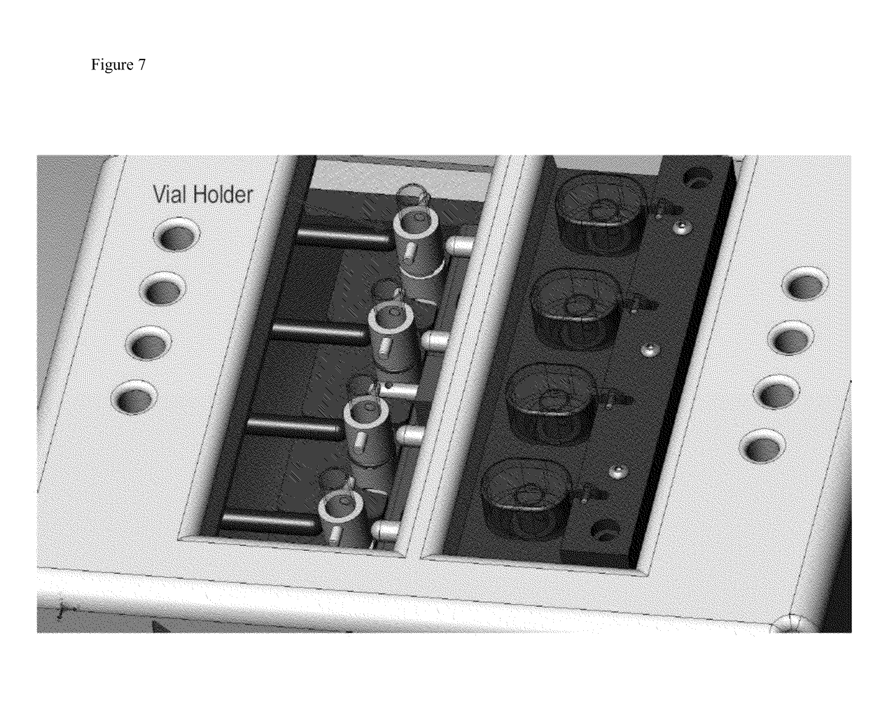

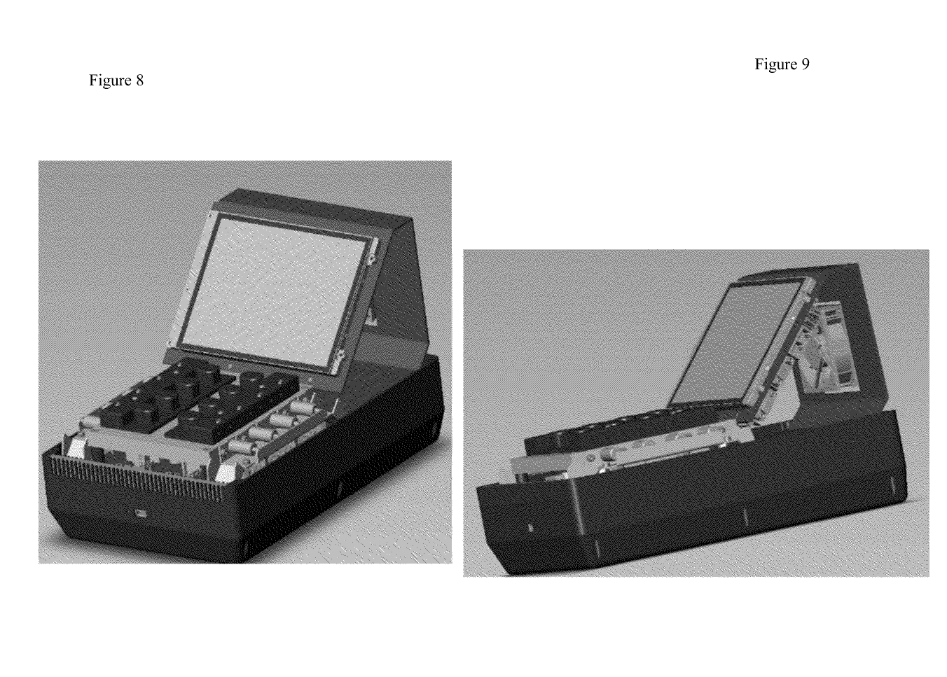

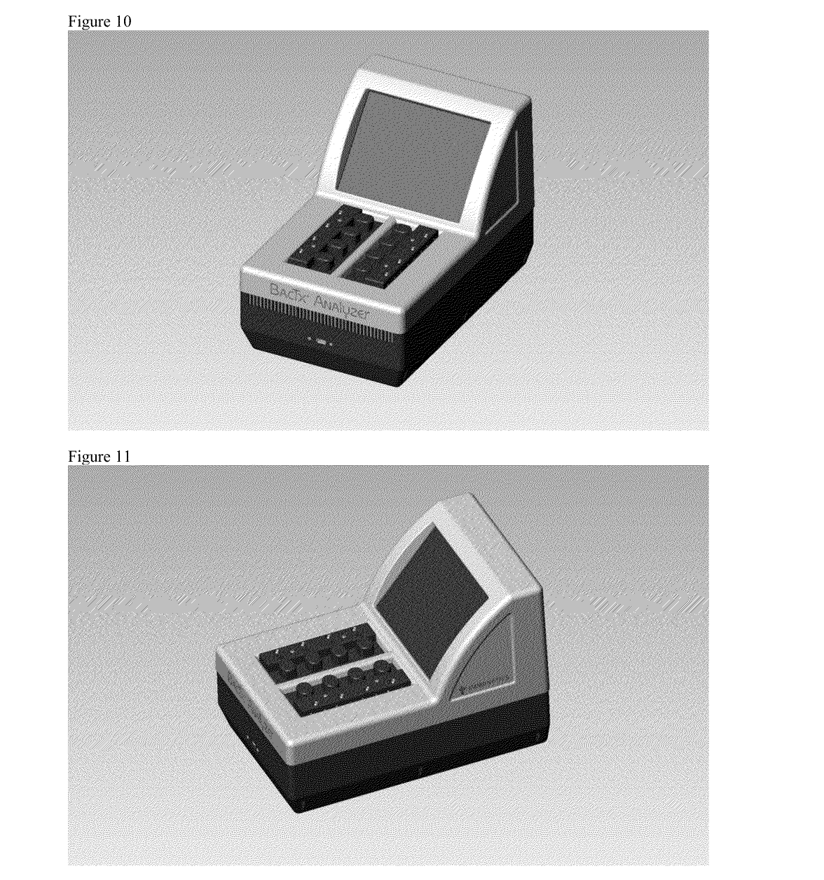

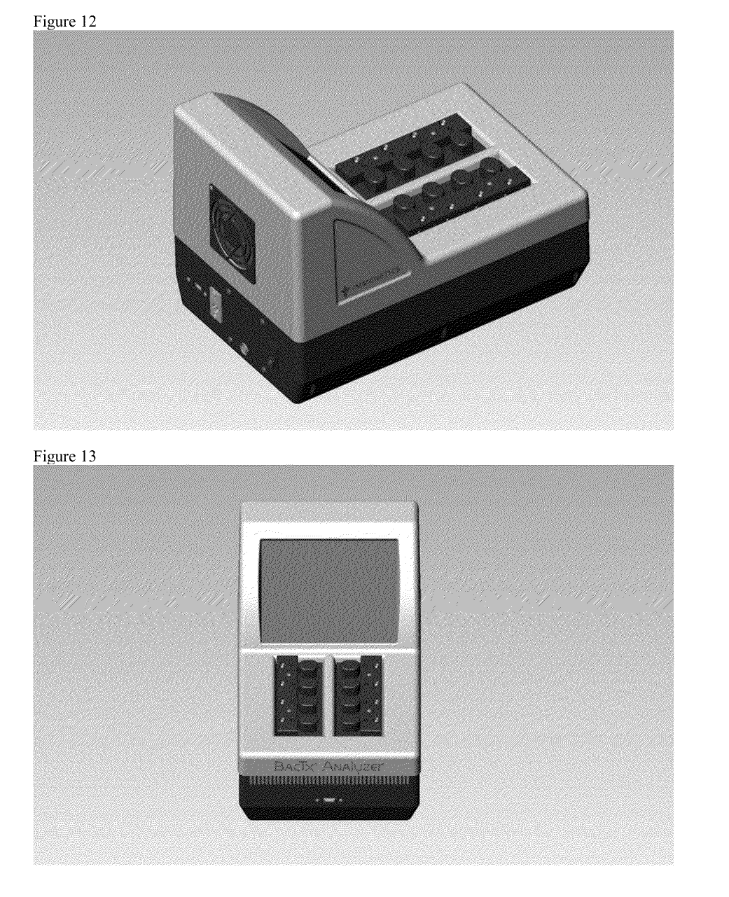

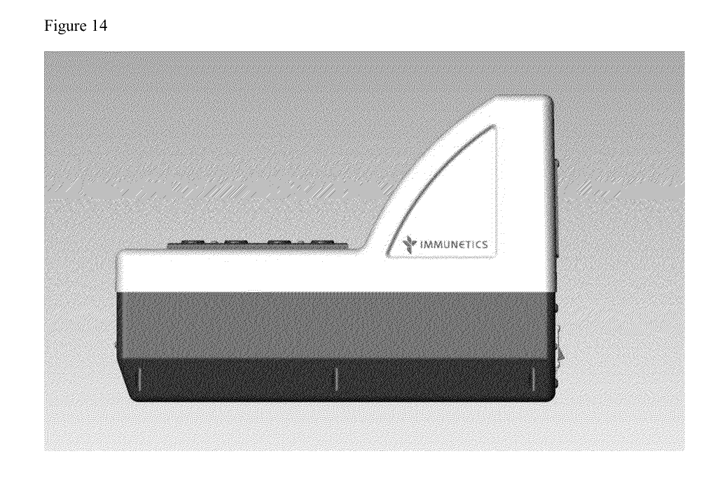

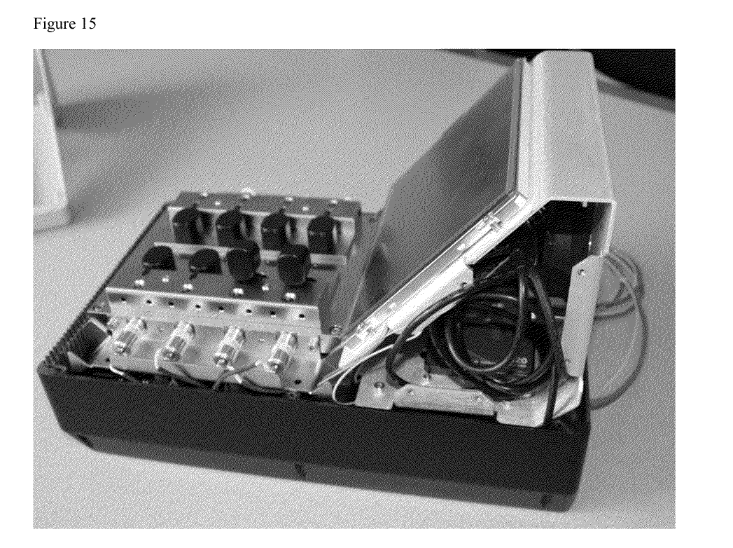

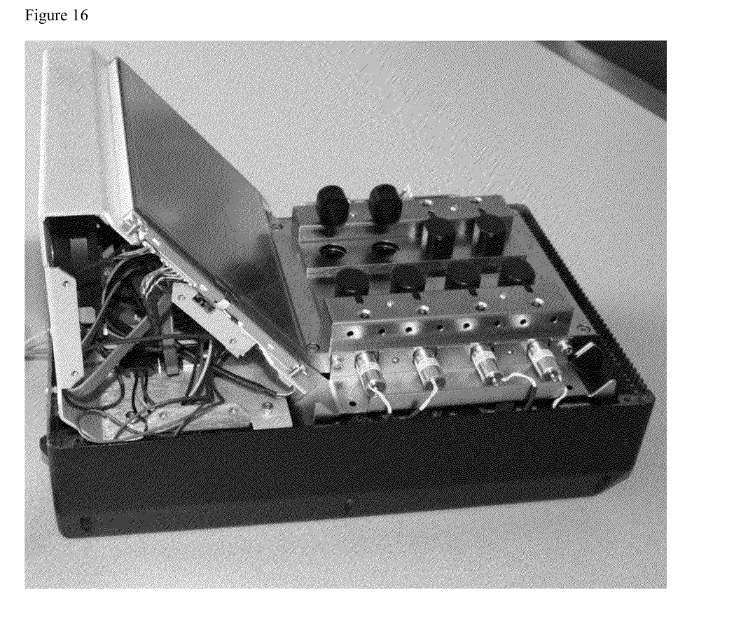

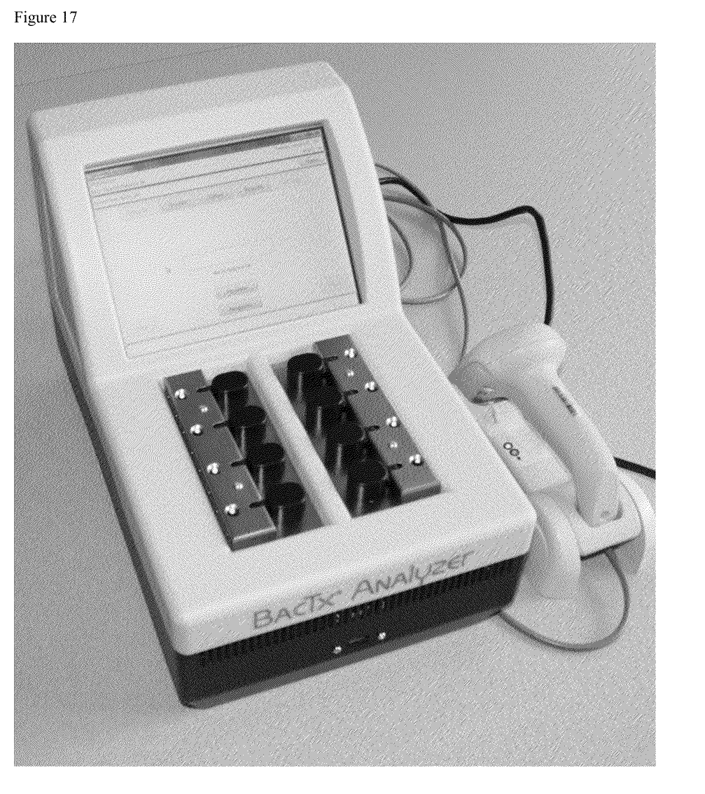

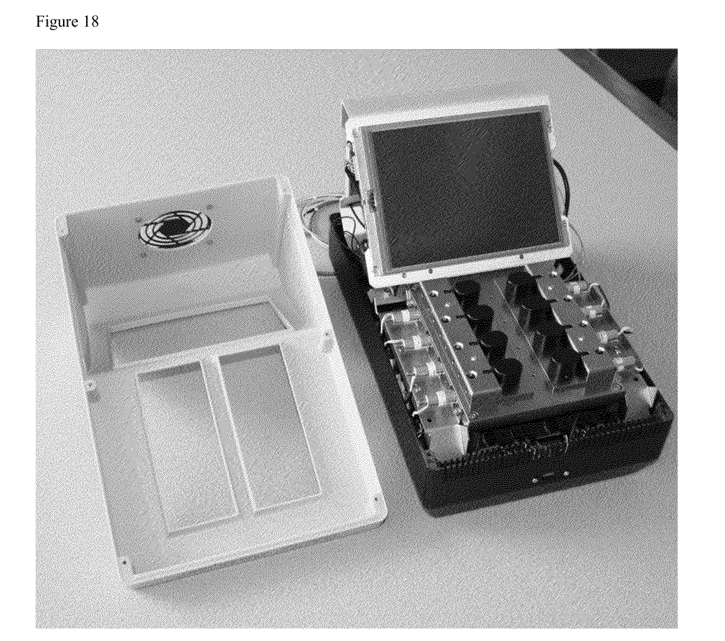

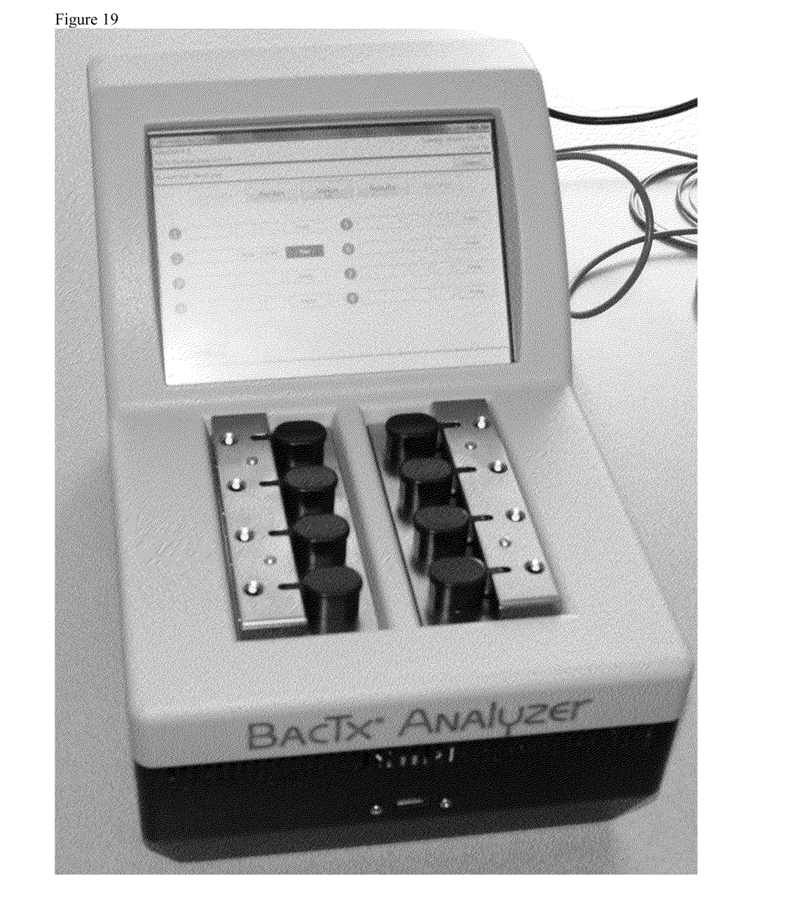

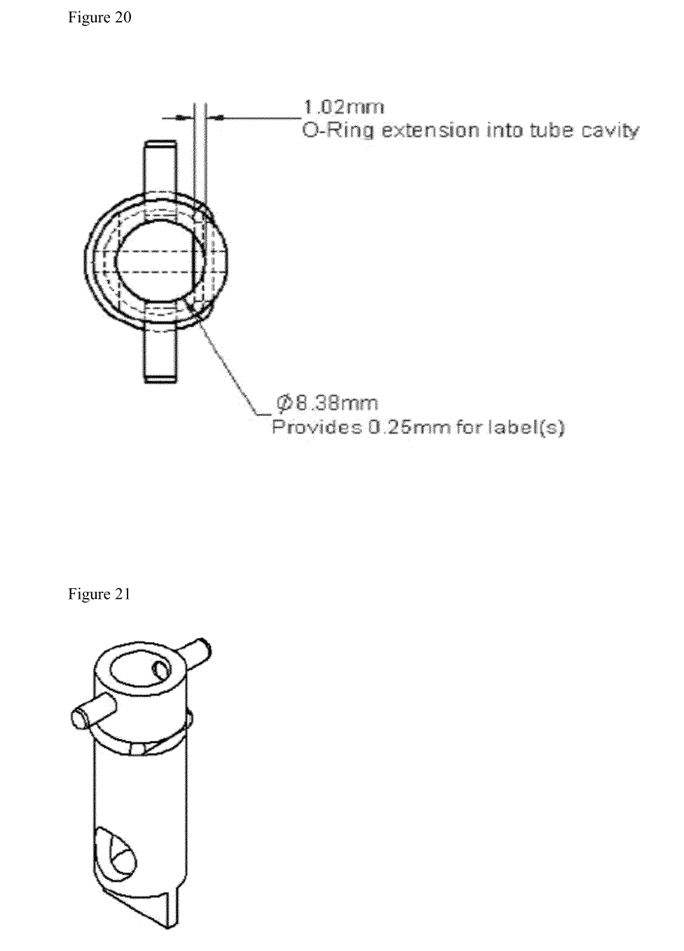

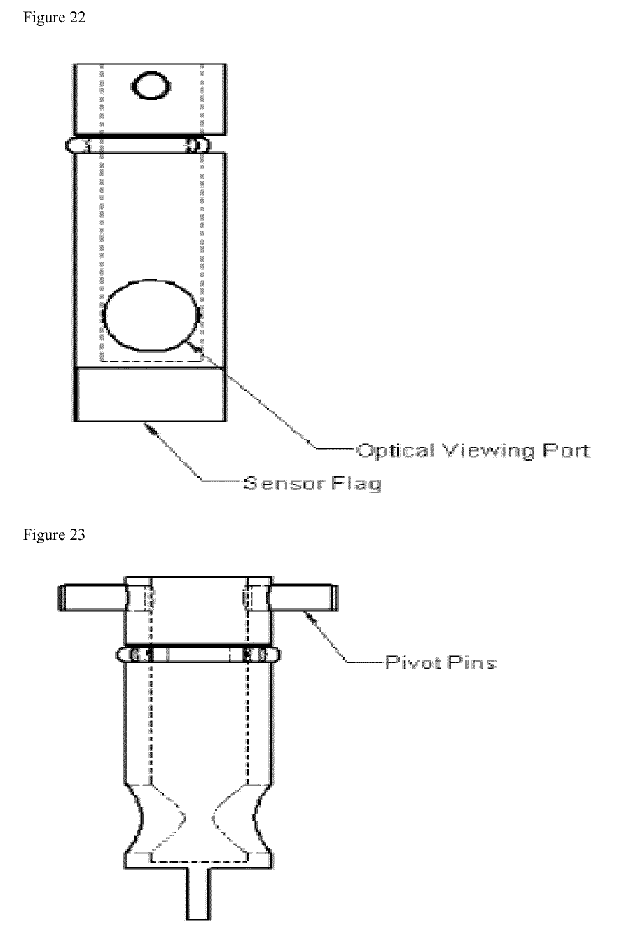

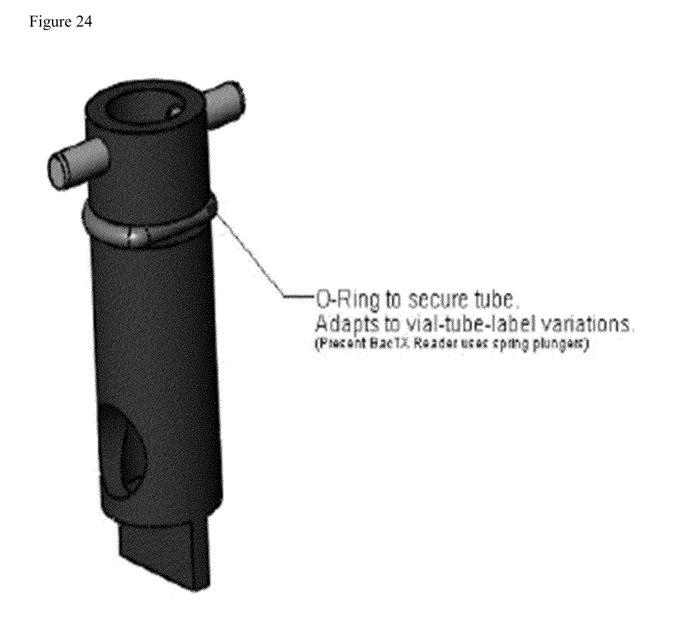

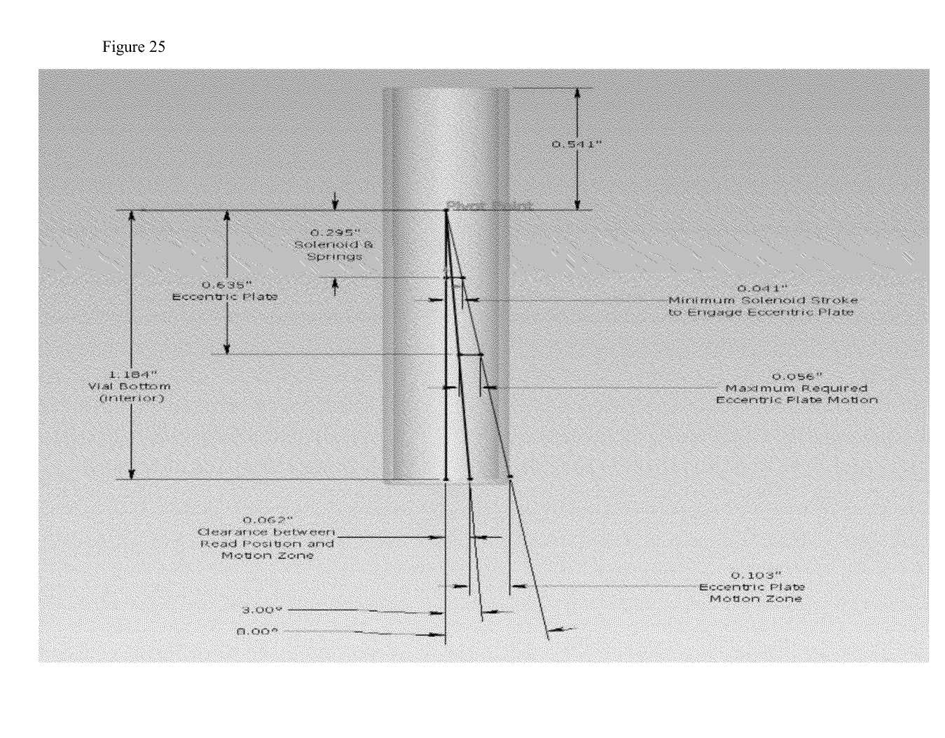

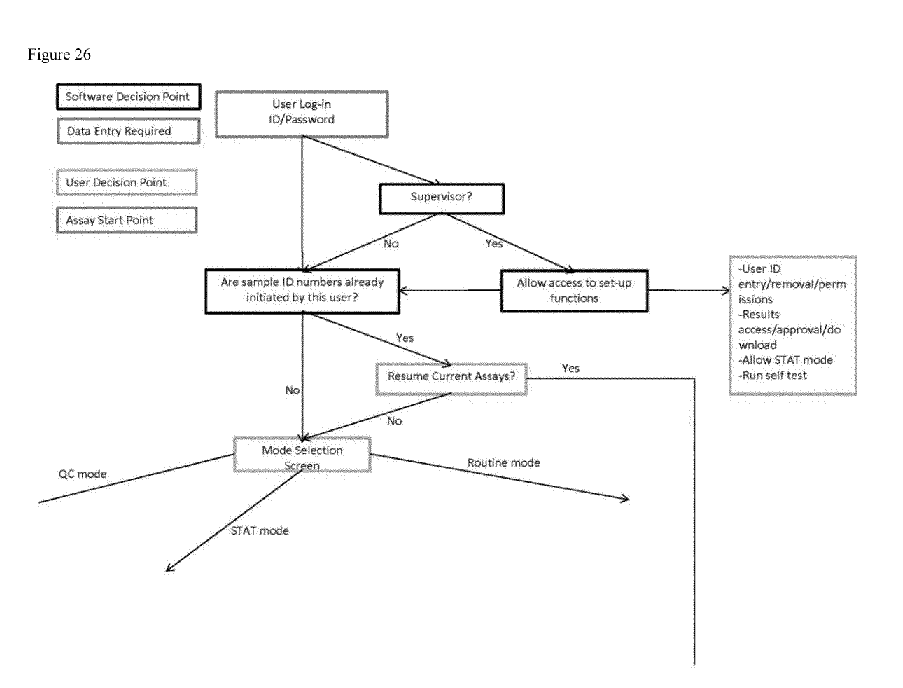

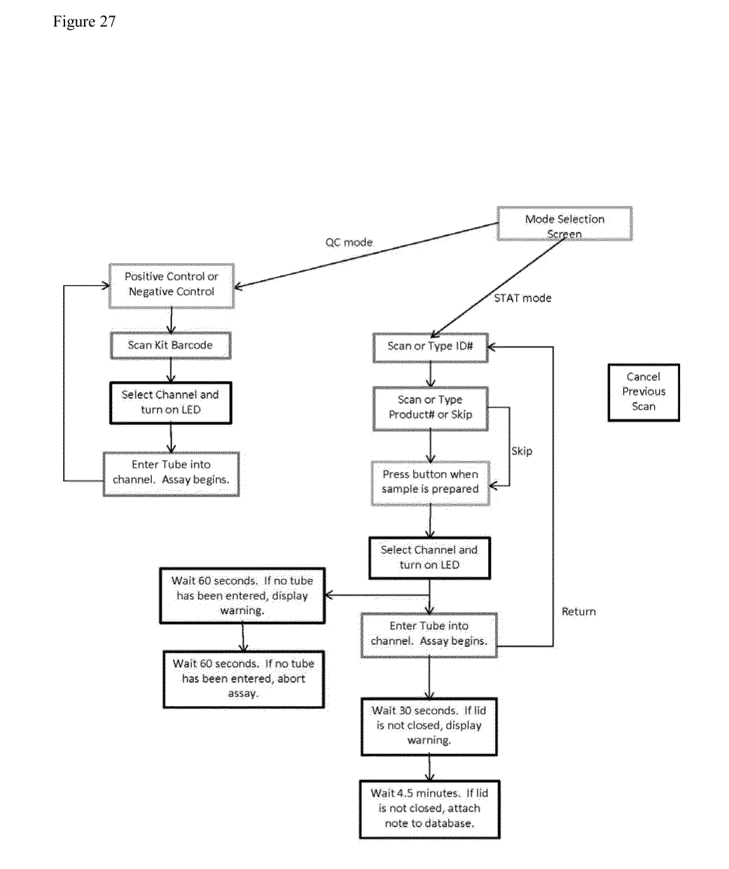

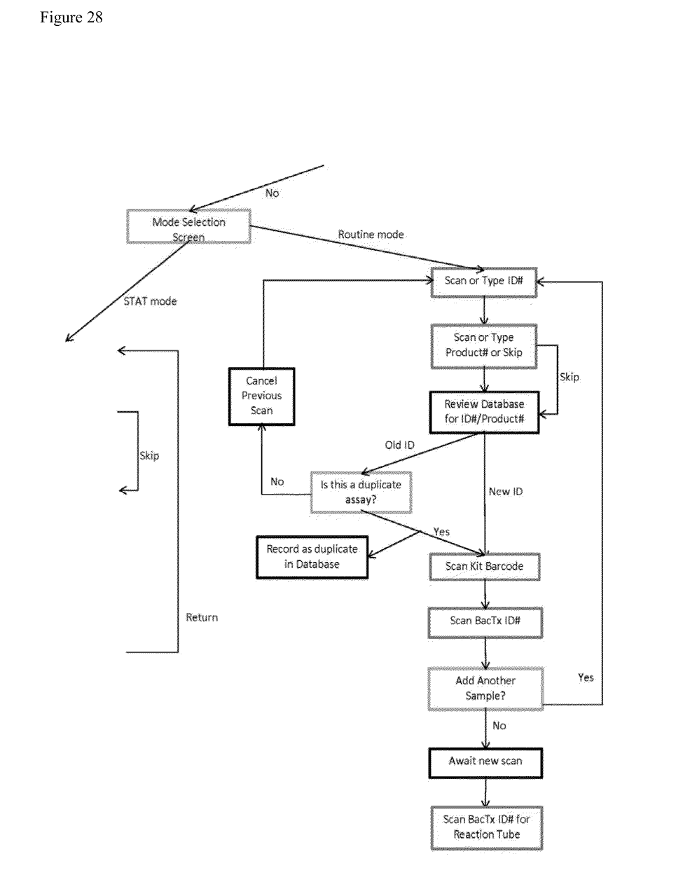

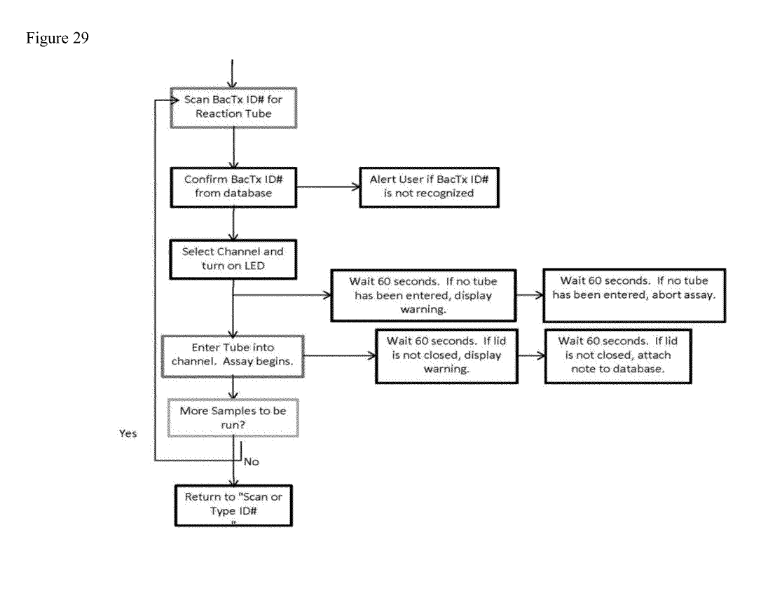

### DETAILED DESCRIPTION OF THE INVENTION

One aspect of the present invention features an optical reader apparatus for monitoring and data collection of colorimetric-based biochemical assays. The reader has an integrated mechanical agitation (sample mixing) function and is capable of measuring optical density of single or multiple samples simultaneously and independently, being fully random-access and asynchronous in operation. The optical system of the reader is solid state, with LED illumination emitting visible wavelengths for transmission through a test sample. Solid state detectors are used to monitor and quantify optical density changes in the sample via the level of light transmitted. The reader's optical system is self-calibrating and reads up to 2.0 standard OD units, and can discriminate between positive and negative samples in an automated fashion.

The reader is equipped with onboard single-board computer and data storage, a touchscreen monitor for user interaction, and standard USB 2.0 ports for connectivity and data retrieval. ISBT Barcodes (1D and 2D formats) can be used to identify and track test samples, by attaching a commercial barcode scanner to the reader through a USB port. Similarly, USB ports can be used to download data onto a flash drive or similar device. The user interface allows multiple simultaneous user logins with security features to protect assays in process and assay data, including screen locking/timeout and PIN-based login. Color coded labels having unique ID numbers, with corresponding onscreen display of sample-specific color is available for enhanced sample ID and tracking.

The assay reader is designed in both hardware and software to maximize utility of the BacTX® assay or similar colorimetric assays, in screening or clinical laboratory settings, and has ease-of-use features to streamline workflow and reduce the possibility of data entry or sample identity errors. The reader and its software are tailored for specific assays and achieve semi-automated operation relative to use of standard laboratory equipment, and consequently the reader requires much less input and hands-on manipulation from the user following an initial sample preparation step.

With specific application to ensuring the safety of platelets for transfusion and for the diagnosis of UTI and bacterial infection of the central nervous system, a rapid, sensitive, and specific assay device and system for the detection of bacteria in samples such as platelet units, urine, and cerebrospinal fluid (CSF) samples has been developed.

### I. Abbreviations:

“GUI” stands for graphical user interface.

“PIC” stands for Product Identification/Information Code. Secondary identifier provided by the platelet manufacturer to represent one of several potential products donated at the time of the DIN being assigned. The PIC is part of the ISBT label on the platelet bag that identifies the product type. Note: platelet bags could have the same DIN but different Product Information Codes. This is also referred to as the Product Code.

“ATP” stands for Manufacturing Acceptance Test Procedure

“DIN” stands for Donor Information Number. The DIN is part of the ISBT label on the platelet bag that uniquely identifies the donor. DIN refers to the unique ID provided by the platelet manufacturer to represent the donor at the time of platelet donation.

“IBC” stands for Interface Board Controller. The IBC is a microcontroller residing on the SCB PCB that interfaces to and controls the external devices, via firmware execution, required for the processing the assays.

“ISBT” stands for International Society of Blood Transfusions or International Standard for Blood and Transplants. ISBT is a system for identification, labeling and processing of human blood, tissue, and cellular therapy products using an internationally standardized system.

“SBC” stands for Single Board Computer.

“SCB” stands for System Control Board; interfaced to the SBC.

“PCB” stands for Printed Circuit Board.

“STAT” refers to the assay processing for a particular sample becomes a priority and must be processed immediately.

“USB” stands for Universal Serial Bus.

### II. Definitions:

“Absorbance” refers to the calculated measure of proportion of light intensity absorbed by a sample, defined as A=−log(I₀/Iₜ), where I₀=light intensity at time zero (initial reading) and Iₜ=light intensity at the current read time.

“ID” refers to a randomly assigned barcode used to track the specific sample being tested. This barcode contains a 3 digit Tag that links to the Reagent ID. The ID can be logically associated in software with the DIN and PIC of the platelet sample unit by using the integrated barcode scanner.

“Barcode” refers to a symbology, numerical, alphabetical, alphanumerical, symbolic, or biometric identification for a reagent ID, BacTx®, platelet unit barcode refers to a code, such as a bar code, that is engraved or imprinted on the reaction tube, reagents, or assay product components. The BacTx® ID, DIN, PIC, and ISBT are exemplified herein, but such exemplification is not intended to limit the intended scope of the disclosure. For example, the symbology is any code known or designed by the user. The symbols are associated with information stored on the on-board computer of the optical reading apparatus or a separate data storage device. For example, each reaction tube can be uniquely identified with an encoded symbology. It is contemplated herein that identifying and other information can be encoded in the barcode, which can be read by the barcode scanner attached to the optical reader apparatus when the reaction tube is read. Alternatively, the barcode or other symbology may be read by any of scanning device known to those of skill in the art.

As used herein, a barcode is a symbology, typically a field of alternating dark bars and reflective spaces of varying widths, that is affixed onto or associated with an item and provides identifying information about the item. Barcodes can placed on a reflective background, and the contrast between the dark bars and reflective spaces, or the reflectivity ratio, allows an optical sensor in a reader to discern the transitions between the bars and spaces in the symbol. Barcodes are electro-optically scanned, typically using a laser or LED, and generate a signal that is transmitted to an associated computer whose memory has digitally stored therein identifying information associated with the item. The item is thereby automatically identified by its barcode and can be tracked, or additional information can be added to the stored information associated with the encoded item.

Several barcode formats are available and are used for different purposes. A number of different bar code symbologies exist, these symbologies include UPC/EAN codes, Code 39, Code 128, Codeabar, Interleaved 2 of 5 and many others; two-dimensional codes, such as PDF 417, Code 49, Code 16K; matrix codes (Data Code, Code 1, Vericod); graphic codes; and any others known to those of skill in the art. Preferred herein are one-dimensional codes, such as the well-known Code 39 and Code 128, although two-dimensional codes (see, e.g., U.S. Pat. Nos. 5,243,655 and 5,304,786, also are suitable for use herein.

The 39 barcode was developed in 1974 to provide a fully alphanumeric bar code for data entry systems. This barcode is especially effective in applications that use alphanumeric data for item identification. The structure of 39 permits it to be printed by a wide variety of techniques, including offset, letterpress, fully-formed impact printers, dot matrix printers, and on-impact printing devices.

Current application areas include inventory control, manufacturing work-in-process, tracking, wholesale distribution, hospitals, government agencies and retail point of sale. Code 39 is the most widely used alphanumeric barcode. It has been accepted as a standard code by many companies and industries. Specification ANSI Draft MH10.X-1981, entitled, “Specifications for Bar Code Symbols on Transport Packages & Unit Loads,” describes three different bar code symbologies. Code 39 is called 3-of-9 code in the ANSI specification. Moreover, the Depae MIL-STD-1189, dated Jan. 4, 1982, defines 39 (called 3 of 9 code) as the standard symbology for marking unit packs, outer containers, and selected documents.

Code 39 includes 9 bits, at least three of which are always 1. Code 39 can be used to encode a set of 43 characters, including upper case alphabetic and numeric (0-9) characters, as well as seven special characters (−, ., *, $, /, + and %). The beginning and end characters are always an asterisk (*). The code uses narrow and wide bars along with narrow and wide spaces, and the encoding for a single character is made up of a pattern of bars and spaces. The code structure is three wide elements out of a total of nine elements, where an element is the area occupied by a bar or space). The nine elements include five bars and four spaces.

In Code 128, every character is constructed of eleven bars and spaces, and all 128 ASCII characters, i.e., numeric characters, upper and lower case characters, punctuation and control codes are encoded. There are three different character sets to select from: one set encodes all upper case characters and all ASCII control characters; another encodes all upper and lower case characters; and the third encodes all numeric characters. Through the use of special characters, it is possible to switch between character sets within a single code symbol. Code 128 uses four different bar and space widths. Each data character encoded in a Code 128 symbol is made up of 11 black or white modules. Three bars and three spaces are formed out of the 11 modules. There are 106 different three bar/three space combinations. Bars and spaces can vary between one and four modules wide. The stop character is made up of 13 modules. The symbol includes a quiet zone (10×-dimensions), a start character, the encoded data, a check character; the stop character and a trailing quiet zone (10×-dimensions) (see, e.g., U.S. Pat. No. 5,262,625).

The term “β-glucan” as used herein refers to β-1,3-glucan, a cell wall component of true fungi such as yeast and mold and a major polysaccharide component of fruit bodies of many basidiomycetes.

The terms “chromogenic phenoloxidase substrate” and “chromogenic substrate” as used herein refer to a substrate of phenoloxidase that generates a colored reaction product. Exemplary chromogenic phenoloxidase substrates are L-3,4-dihydroxyphenylalanine, 3,4-dihydroxyphenethylamine; (dopamine), 3,4-dihydroxyphenyl propionic acid, 3,4-dihydroxyphenyl acetic acid, or catechol.

“Demonstration” as used herein, is a term referring to a method of verification that is limited to readily observable functional operation to determine compliance with requirements. This method shall not require the use of special equipment or sophisticated instrumentation.

The term “hemolymph” as used herein refers to body fluid or plasma obtained from the hemocoel (primary body cavity) of an insect. Hemolymph may be isolated using the methods disclosed by Ashida in Insect Biochem. 11, 57-65 (1981), U.S. Pat. Nos. 4,970,152, 5,585,248, or 5,747,277. Hemolymph may be isolated from insects belonging to the orders including, but not limited to Lepidoptera (such as Manduca sexta (tobacco hornworm), Manduca quinquemaculata (tomato hornworm), Galleria mellonella, Hyalphoma ceropia, Bombyx mori (silkworm)), Diptera (such as Sarcophaga peregrina (flesh fly), Sarcophaga mucosa, Mucsa domestica (house fly)), Orthoptera (such as Locusta migratoria, Teleogryllus (e.g., Emma field cricket), Coleoptera (beetles) (such as Cerambyx and Acalolepa luxuriosa). Insects may be used at any stage of development and thus may be larvae or adult. Hemolymph isolated from insects may comprise peptidoglycan-binding proteins. The assay methods utilize a prophenoloxidase cascade system isolated from the hemolymph or plasma of the silkworm larvae, Bombyx mori. β-glucan may be detected using hemolymph or plasma from insects. β-glucan may be detected using the hemolymph or plasma of the silkworm larvae, Bombyx mori.

“Inspection” as used herein, is a term referring to a method of verification consisting of investigation, without the use of special laboratory appliances or procedures, to determine compliance with requirements. Inspection is generally nondestructive and includes (but is not limited to) visual examination, manipulation, gauging, Certificate of Compliance from the supplier, and measurement.

“Light source” as used herein may refer to full-spectrum, ultraviolet, visible, infrared, light emitting dioded (LED), or near infrared. The light source may include a photodetector adapted to read a reaction tube using reflected light, including fluorescence, or electromagnetic radiation of any wavelength. Reflectance detector can be detected using a photodetector or other detector, such as charge coupled diodes (CCD), silicon photodiode detector, gamma detector, Photodiode, Photo multiplier tube, IR-NIR arrays, Focal Plane Array, InGaAs photodetector; VisGaAs photodetector, InSb photodetector, Quantum Well Infrared photodetector, or combinations thereof. The light source may further included light-emitting diodes, optical fibers, a sensing head, including means for positioning the sensing head along the reaction tube, a control circuit to read the photodetector output and control the on and off operation of the light-emitting diodes, and a memory circuit for storing raw and/or processed data. The light source may further comprise filters, diffusers etc., which can be stationary or have the ability to move in any directions or angles, depending on how the user would desire the system to be configured. Examples of different configurations of the system comprise: stage movable, lens and/or sensor fixed; stage fixed, lens and/or sensor movable, lens fixed, sensor &/or stage movable; epi-illumination imaging, trans-illumination imaging, split-beam dual detector systems, diffuse axial illumination imaging, directional illumination imaging, glance illumination imaging, diffuse illumination imaging, darkfield illumination imaging, backlighting illumination imaging or any combinations thereof.

The term “L-3,4-dihydroxyphenylalanine” or “DOPA” refers to a phenoloxidase substrate. Quinones produced by phenoloxidase action on DOPA or another substrate may be detected as a colored complex with 3-methyl-2-benzothiazolinone hydrazone (MBTH) or derivative thereof. DOPA is also a chromogenic reagent that in turn may be converted into a colored melanin reaction product. The black melanin reaction product can be detected visually or spectrophotometrically at an absorbance in a wide range of wavelength. Absorption at 650 nm is typically used for detection of the melanin polymer.

The term “3-methyl-2-benzothiazolinone hydrazone” or “MBTH” refers to a chromogenic reagent that produces stable colored adducts with quinones. This reaction product can be detected visually or spectrophotometrically. Quinone-MBTH complexes are soluble and have an absorption maximum in a range of 450-510 nm depending on the substrate producing the quinone. Quinone-MBTH complexes visually have a red color. Spectrophotometric methods for determining phenoloxidase and tyrosinase activity using MBTH are described in Rodiquez-Lopez et al., Anal. Biochem. 216:205-12 (1994) and Winder, A. J., J. Biochem. Biophys. Methods 28:173-183 (1994).

The term “3-methyl-2-benzothiazolinone hydrazone derivative” or “MBTH derivative” refers to various compounds having the general structure:

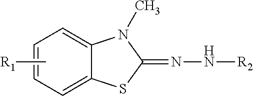

wherein R₁ represents H, alkyl, halide, —NO₂, —CO₂, or —SO₃; and;

R₂ represents H, or —SO₂R₃;

wherein R₃ represents alkyl, aryl, and heteroaryl.

The term “peptidoglycan” as used herein refers to a glycopeptide polymer that is a component of bacterial cell walls, including Gram-positive and Gram-negative bacteria. Peptidoglycan is generally characterized as containing N-acetylglucosamine or N-acetylmuramic acid and D- and L-amino acids.

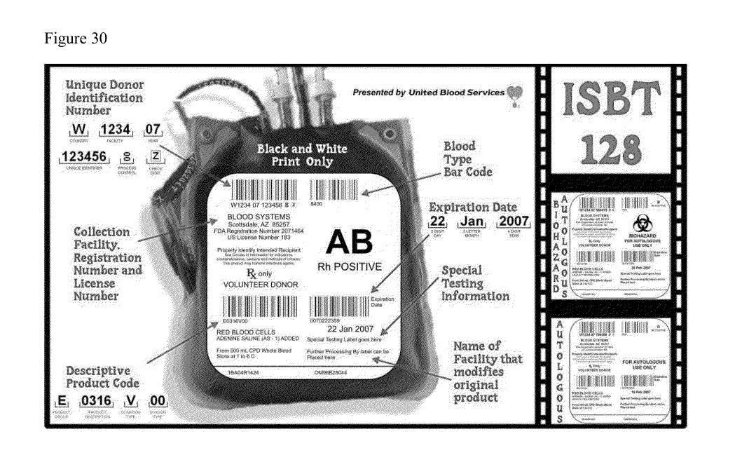

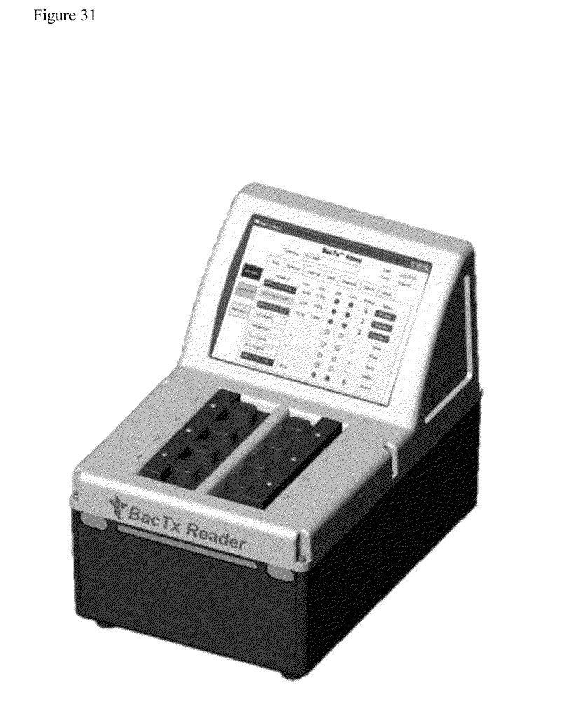

“Platelet Unit Barcode” refers to the ISBT 128 barcode and can be defined as the international standard for the transfer of information associated with human tissue transplantation, cellular therapy, and blood transfusion. It provides for a globally unique donation numbering system, internationally standardized product definitions, and standard data structures for bar coding and electronic data interchange. By complying with ISBT 128, collection and processing facilities provide electronically readable information that can be read by any other compliant system. An example of an ISBT barcode is shown in FIG. 30.

The term “prophenoloxidase cascade system” or “pro-POC system” as used herein refers to a serine proteinase cascade system that is present in the hemolymph and cuticle of the body wall of insects. A prophenoloxidase cascade system comprises a prophenoloxidase activating enzyme, prophenoloxidase, and a serine proteinase cascade. A pro-POC system may further comprise a peptidoglycan-binding protein(s) (PGBP) and/or a β-glucan-binding protein(s) (BGBP). The prophenoloxidase cascade system may additionally comprise components that remain to be identified. The prophenoloxidase cascade system from silkworm larvae plasma, however, represents a complete prophenoloxidase cascade system. In nature, the prophenoloxidase cascade system is one of the immune mechanisms in insects and is triggered by injury or minute amounts of peptidoglycan or β-glucan. Activation of the cascade begins from a specific recognition of peptidoglycan (PG) or β-1,3-glucan with a corresponding PGBP or BGPB. These specific complexes trigger a serine protease cascade which activates prophenoloxidase activating enzyme, a specific protease, which in turn activates prophenoloxidase through cleavage of an N-terminal portion of this enzyme, which generates phenoloxidase, the active form. Active phenoloxidase catalyzes two reactions: 1) the oxidation of monophenols to o-diphenols and 2) the oxidation of o-diphenols to quinones. Quinones produced by the action of phenoloxidase on L-3,4-dihydroxyphenylalanine (DOPA) may non-enzymatically polymerize the formation of a black melanin polymer. A prophenoloxidase cascade system may be obtained from silkworm larvae plasma as described by Ashida in Insect Biochem. 11, 57-65 (1981) or U.S. Pat. No. 4,970,152.

“Product Information Code (PIC)” refers to each platelet unit is assigned a product information number to identify individual product types that may be collected from a single donor (and therefore share a DIN). For apheresis platelets, this allows the user to recognize the difference between multiple bags collected at the same time.

“Reagent ID” refers for barcode present on exterior of the BacTx® Kit box that represents the unique lot number.

“Reaction tube” as used herein is the disposable assay tube which is inserted into the sample vial holder.

“Record” refers to data storage for operational parameters and/or states.

“Sample” as used herein may refer to biological or clinical samples that may be tested for bacterial and/or fungal contamination include, but are not limited to blood, blood products, platelet units/collections, platelet concentrates, serum, plasma, other blood fractions, tissue, tissue extracts, urine, lymph, hydration fluid (i.e., IV hydration fluids), dialysis fluid, cerebrospinal fluid (CSF), nutrient fluid, vaccines, anesthetics, pharmacologically active agents, stem cells for transplant, or imagining agents. Wound dressings may also be tested for bacterial and/or fungal contamination. A sample may be a suspension or a liquid. Bacteria or fungi present in the sample may be collected and optionally concentrated by centrifugation or filtration, but not in combination. Alternatively, the sample may be dried or evaporated. In addition, agricultural products, environmental products, and manufacturing products, including process samples, may be tested for bacterial and/or fungal contamination using the assay method. Non-limiting examples of agricultural products include food products and the water supply. Testing of the water supply may be extended from water that is consumed by humans and other animals to water that is used in recreational facilities including swimming pools and lakes. Non-limiting examples of environmental products include machinery that is used for processing a wide array of samples and products consumed and used by humans. Non-limiting examples of manufacturing samples include sterile products and their components and intermediates that are manufactured for medical uses.

“Silkworm larvae plasma (SLP)” is available commercially from Wako Chemicals, Inc, Richmond, Va. The technology of measuring peptidoglycan or β-glucan in an assay using SLP is covered by U.S. Pat. Nos. 4,970,152, 5,585,248, 5,747,277, 6,034,217, and 6,413,729 issued to Ashida et al., of Japan and is described in Kobayashi et al., FEMS Immunol. Med. Microbio. 28:49-53 (2000). The assay methods comprise a fraction obtained from the hemolymph (plasma) of an insect, such as a silkworm, which is capable of specifically reacting with peptidoglycan or β-glucan, and the production of purified recombinant peptidoglycan binding proteins.

“Test” as used herein, is a term referring to a method of verification that employs technical means, including (but not limited to) the evaluation of functional characteristics by use of special equipment or instrumentation, simulation techniques, and application of established principles and procedures to determine compliance with requirements.

“Timeout” refers to the maximum time over which absorbance is read for a given assay tube.

“Unique (DIN)” refers to a 13 character identifier built up from three elements, the first identifying the collection facility, the second the year, and the third a sequence number for the donation. For example: G151710600001 where: G1517 identifies the collection facility; 10 identifies the collection year as 2010; 600001 is the sequence number of the donation assigned by the collection facility. The two digits printed vertically allow individual bar codes in a number set to be discreetly identified hence providing an option to add process control into the collection process. An additional character is enclosed in a box at the end of the identifier. This is a checksum character used when a number is entered into a computer system through the keyboard to verify the accuracy of the keyboard entry.

### III. Assay Methods

Any colorimetric assay may be used in conjunction with the optical reader apparatus and assay systems of the present invention, such as any chemical assays, nucleic acid assays, fluorometric assays, chemiluminescent, bioluminescent assays, or ligand-based assays which may be adapted for detection in the assay device of the present invention. The preferred colorimetric assay method is the BacTx® assay method (described in U.S. Pat. Nos. 7,598,054 and 8,450,079, each of these patents are herein incorporated by reference). The assay method is a rapid, enzyme-based, chromogenic assay that detects peptidoglycan, a universal component of both gram-positive, gram-negative, aerobic, and anaerobic bacterial cell walls. Thus, peptidoglycan provides a useful broad-spectrum marker for the presence of microorganisms, such as pathogens, in samples. The assay method enables measurement of peptidoglycan either quantitatively or qualitatively, in either the presence or absence of other sample components, such as platelets. Peptidoglycan may be detected using hemolymph (plasma) from invertebrates. Peptidoglycan may be detected using plasma or hemolymph from insects.

The assay method detects peptidoglycan and is thus also distinct from two FDA approved automated platelet culture systems currently available. One conventional system, the Pall BDS, uses changes in oxygen concentration as a result of bacteria growth to provide a practical and reliable test. Since aerobic bacteria consume oxygen, abnormally low levels of oxygen in a platelet sample indicate the presence of bacteria. A small volume of platelet concentrate is incubated with an agent to promote the growth of a wide variety of bacteria species. Oxygen levels are measured and a simple pass or fail reading is obtained (Yomtovian, R. et al. (2001) AABB corporate evening Symposium; October 15). A second currently available system, the BioMerieux BacT/ALERT®, automatically detects the presence of bacteria by tracking their production of CO₂. A sensor at the bottom of a culture bottle containing the specimen indicates the presence of CO₂ by changing color, from gray to yellow (Brecher et al. (2002) Transfusion 42:774-779). Both of these systems require secondary instrumentation for sample analysis and require up to 30 hours for bacterial culture. See Table 1 for method comparison data.

TABLE 1
Comparison of Pall BDS and BacT/ALERT ® Methods

| | Pall BDS | BacT/ALERT ® |
|---|---|---|
| Detection Method | O₂ Depletion | CO₂ Production |
| Negative Predictive | | 99.97% |
| Value | | |
| Specificity | 100% | 99.8% |
| Sensitivity | 95.8-100% | |
| Assay Time | 24-72 hours | 9.2-26 hours |
| Sample Type | Whole blood/apheresis | Cleared for apheresis and |
| | platelets | whole blood platelets |

A third available system Verax Biomedical PGD Platelet® uses an immunoassay to detect bacterial contaminants.

In contrast, the assay methods utilize in the present invention detects peptidoglycan or β-glucan directly. Peptidoglycan is detected on contaminating bacteria. Contaminating bacteria may be Gram-positive and/or Gram-negative bacteria. Non-limiting examples of bacteria that may be detected in contaminated platelet units include Proteus vulgaris, Yersinia enterocolitica, Serratia marcescens, Enterobacter cloacae, Staphylococcus epidermidis, Staphylococcus aureus, Klebsiella pneumoniae, Bacillus cereus, Escherichia coli, Proteus mirabilis, Pseudomonas aeruginosa, and Salmonella cholerae. Bacteria may represent common skin flora, as listed above, as well as normal and pathogenic gut flora. Examples of pathogenic gut bacteria include, but are not limited to, strains of Salmonella, Shigella, Campylobacter, Yersina, Vibrio, Caostriduim difficile, and Escherichia coli. Other non-limiting examples of bacteria that may be detected using the assay method include a member of the genus Escherichia, Streptococcus, Staphylococcus, Bordetella, Corynebacterium, Mycobacterium, Neisseria, Haemophilus, Actinomycetes, Streptomycetes, Nocardia, Enterobacter, Yersinia, Fancisella, Pasteurella, Moraxella, Acinetobacter, Erysipelothrix, Branhamella, Actinobacillus, Streptobacillus, Listeria, Calymmatobacterium, Brucella, Bacillus, Clostridium, Treponema, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, and Borrelia.

Bacteria may be detected in the assay protocol as colony forming units (CFU)/ml as low as about 100 CFU/ml, e.g., about 100-200 CFU/ml, about 200-300 CFU/ml, about 300-600 CFU/ml, 600-1000 CFU/ml, about 1000-2500 CFU/ml, 2500-5000 CFU/ml or 5000-10,000 CFU/ml. The CFU/ml of bacteria detected in platelets will depend on the identity of the bacteria and the length of bacterial contamination. Bacterial species including both Gram-positive and Gram-negative bacteria may be detected at concentrations of approximately 100 CFU/ml, which is similar to the range detected by longer, more conventional culture procedures.

The assay methods may be used to detect β-glucan, a cell wall component of fungi, such as yeasts and molds. Yeast and other fungal cells include, but are not limited, to the genus Acremonium, Alternaria, Amylomyces, Arthoderma, Aspergillus, Aureobasidium, Blastochizomyces, Botrytis, Candida, Cladosporium, Crytococcus, Dictyostelium, Emmonsia, Fusarium, Geomyces, Geotrichum, Issatchenkia, Microsporum, Neurospora, Oidodendro, Paecilomyces, Penicillium, Pilaira, Pityrosporum, Rhizopus, Rhodotorula, Saccharomyces, Stachybotrys, Trichophyton, Trichoporon, and Yarrowia.

The assay method may be used in conjunction with the optical reader apparatus which reads the colored reaction product and provides a rapid and cost-effective approach to screening platelet units for bacterial and fungal contamination. The optical reader apparatus will generate and output indicating a positive or negative reading of bacterial or fungal contamination in the sample.

The features and benefits of the assay method include a sensitivity-detection of common pathogens to less than or equal to about 100 CFU/ml, a specificity of about 100%, a short assay time and the option of immediate readout using visual evaluation. The flexible format and simplicity of the assay lends itself easily to laboratory automation for batch testing in the blood bank or point of use, e.g. testing in the hospital, doctor's office, testing facility, clinical laboratory, manufacturing plant, or in the field (depending of course on the sample to be evaluated). Thus, the bacterial detection assay format is simple and straightforward.

The assay method is a kinetic assay. The assay is not a metabolic assay and differs from other assay protocols that are currently available. BacT/ALERT® and BDS systems look for metabolic signatures (markers of respiration). The assay method is amendable to testing about 1, 5, 10, 100, 500 or more samples. A maximum of eight samples may be tested asynchronously or simultaneously using the optical reader apparatus of the present invention. Use of non-sterile equipment is more cost efficient and allows the assay to be more transportable to non-clinical settings.

The assay method may be conducted in less than 1 hour, about 1-2, about 2-3, about 3-4, about 4-5, about 5-6, or about 6-7 hours. In exemplary embodiments, the assay may be conducted in about 30-45 minutes. Assay times over one hour may be required for slow growing bacteria such as S. epidermidis.

Peptidoglycan or β-glucan may be detected in a sample comprising incubating the sample with a prophenoloxidase cascade system, a phenoloxidase substrate that generates a quinone reaction product, and 3-methyl-2-benzothiazolinone hydrazone; and, detecting the formation of a colored prophenoloxidase reaction product, wherein formation of the reaction product indicates the presence of peptidoglycan or β-glucan in the sample. The formation of a colored reaction product further indicates the presence of bacteria or fungi in the sample.

The prophenoloxidase cascade system comprises a phenoloxidase activating enzyme, prophenoloxidase, and a serine proteinase cascade. In further embodiments, the prophenoloxidase cascade system may comprise a peptidoglycan binding protein or a β-glucan binding protein. A prophenoloxidase cascade system may be obtained from insect hemolymph or plasma. In an exemplary embodiment, a prophenoloxidase system is obtained from silkworm larvae plasma.

A phenoloxidase substrate that generates a quinone reaction product may be L-3,4-dihydroxyphenylalanine, L-3,4-dihydroxyphenolamine (dopamine), 3,4-dihyroxyphenyl propionic acid, 3,4-dihydroxyphenyl acetic acid, or catechol. A phenoloxidase substrate that generates a quinone reaction product is L-3,4-dihydroxyphenylalanine (DOPA) or 3,4-dihydroxyphenethylamine (dopamine).

Purified, partially digested peptidoglycan may be used as a standard. Further, a standard curve of peptidoglycan may be constructed by serially diluting purified peptidoglycan from about 10 ng to about 150 pg/ml in extracted and neutralized platelets. Approximately 200 μl of each dilution in extracted and neutralized platelets is incubated with SLP or a reconstituted PCS and incubated at room temperature for up to one hour.

Peptidoglycan, either in a platelet/bacterial sample or as a standard, may be detected in the assay at concentrations as low as about 0.156 ng/ml, and may range from about 0.100-0.200 ng/ml, 0.200-0.500 ng/ml, 0.500-1 ng/ml, 1-2.5 ng/ml, 2.5-5 ng/ml, 5-10 ng/ml, and 10-100 ng/ml. The concentration of peptidoglycan will be proportional to an absorbance reading at about 490 nm, corrected by the subtraction of background, read at 650 nm.

The colorimetric reaction is based on a coupling reaction between o-quinones produced from phenoloxidase o-diphenoloic substrates during the enzyme reaction and 3-methy-2-benzothiazolone hydrazone (MBTH). The MBTH-quinone complex is chromogenic and yields a bright red-colored reaction product that may be measured visually or spectrophotometrically. The reaction product has an absorbance maximum in the range of about 470-510 nm and a high molar absorbance coefficient in the range of 27,500-32,500 M⁻¹ cm⁻¹. Further, the products that are produced in the colorimetric reaction of MBTH with o-quinones are soluble and stable in acidic pH. Thus, the reaction may be stopped with acid, but need not be stopped, and centrifuged to remove aggregated material without significant loss of absorbing material in the supernatant. The cleared supernatant may be measured conveniently using photometric readers, such as spectrophotometers and ELISA readers or by simple visual examination. MBTH adducts in acidic conditions have slightly higher molar absorbency. Replacement of detection methods based on measuring melanin formation in a colorimetric reaction with a MBTH adduct has resulted in a 7 to 10 fold increase in the analytical sensitivity for detection of phenoloxidase activity. Further, by using a reference filter at 650 nm in combination with an analytical filter between 450 and 510 nm, an additional correction for low level residual light scattering can be made.

The assay method described above utilizes a centrifugation step and subsequent extraction step to separate platelets and any contaminating bacteria from plasma containing inhibitory components or non-specific activating substances/factors that may interfere with the SLP test. The extraction procedure removes non-specific activating substances/factors of plasma and simultaneously solubilizes platelets and bacterial cells, thus reducing the turbidity of the solution. The extraction procedure may be adapted to remove any inhibitory factors. Reduction of turbidity in the solution increases the accuracy of the sample readout. This is a significant improvement over other assay protocols that are currently available. In such protocols, the presence of particles, non-specific activating substances/factors, or inhibitory factors in the samples can easily lead to precipitation in the absence of agitation and can alter the measurement by increasing the turbidity leading to a false positive result. Previous attempts by others to eliminate non-specific activating substances/factors or inhibitory factors used extensive dilutions (e.g., 8 to 20 times) that resulted in a decrease in the sensitivity of bacterial detection.

The assay method may employ semi-selective porous physical barrier (filtration), as an alternative concentration step in the sample preparation, to separate bacteria in a sample from other components in the sample that may interfere with the assay. The sample may be filtered through a sterile filter to trap the bacteria present in the sample, the filter rinsed with a solution such as an alkaline solution, and then the filter back-flushed with a rinse solution such as an alkaline solution, thereby eluting the bacteria trapped on the filter. The eluted bacteria may be further processed as to detect the bacteria present in the sample.

The extraction step is an alkaline extraction. In certain embodiments, alkaline extraction may be performed at room temperature or an elevated temperature. Alkaline extraction, as practiced herein, results in approximately a 10-fold concentration of bacterial contaminants since the platelet/bacteria pellet may be prepared from 1 ml solution of the original platelet preparation, and can be efficiently extracted with 100 μl of sodium hydroxide solution. Further, as desired, a greater or lesser-fold concentration can be achieved. Moreover, alkaline extraction can significantly increase the accessibility of peptidoglycan from bacterial cell wall and can partially hydrolyze peptidoglycan polymer generating fragments, which are more accessible substrates for the prophenoloxidase cascade system. As a result, amplification in the sensitivity of detection of contaminating bacteria in platelet samples may be achieved through the extraction step.

Further, alkaline extraction alters the absorption spectrum of hemoglobin, which can be present as a contaminating factor in some platelet preparations. The alkaline extraction procedure shifts the absorbance of hemoglobin minimizing the overlap in absorbance with the MBTH reaction products.

Alkaline extracted platelets are neutralized with an acid buffering system prior to testing with the SLP reagent. In preferred embodiments, the acid buffering substance is MES containing MBTH reagent in an amount equal to the volume of sodium hydroxide solution used for extraction. A stable lyophilized form of MES/MBTH, which can be reconstituted in water on the day of testing, has been developed. Neutralization of the extracted platelets may be performed to optimize the pH and MBTH concentration for the SLP detection step. Neutralization may be performed with as little as a two-fold dilution of the concentrated platelet extract. The final concentration of platelets in an extracted and neutralized sample is five times that in the original platelet sample preparation. For example, in a typical assay, an aliquot of extracted and neutralized platelets (about 100-200 μl) may be added to a tube containing lyophilized SLP reagent and substrate (DOPA or DOPA/dopamine mixture). The reaction may proceed at 37° C. or room temperature for a sufficient period of time to observe a color change (e.g., 60 minutes or less). The samples may be read using a single filter or a two filter approach at 490 nm and 650 nm, as described above. Further, simple visual measurements may be made since a difference in color is used to determine a positive or negative result. The sample color is stable for at least several hours when DOPA is used as a substrate.

Platelets and any contaminating bacteria may be extracted using alternate approaches. Alternate extraction approaches include, but are not limited to, enzymatic extraction.

The binding of a peptidoglycan-binding protein to peptidoglycan may be leveraged though an enzymatic method, as binding triggers a prophenoloxidase enzymatic cascade in the assay system, which utilizes L-3,4-dihydroxyhenylalanine (DOPA) as a phenoloxidase substrate, which in turn may be measured as a colored melanin end product. The colored melanin product is chromogenic and may be measured by visual inspection or through an optical readout.

The pelleted platelets and any bacterial contaminants (natural or spiked) may be collected by dilution with water and centrifugation. Pelleted platelets may be resuspended in water for testing in a silkworm larvae plasma (SLP) reaction.

The foregoing exemplary method may be adapted with no more than routine experimentation for the detection of fungi, contaminants, diagnostic markers, analytes and the like. β-glucan may be detected on the cell wall of fungi. The detection of β-glucan in a platelet sample would indicate that the sample is contaminated with a fungus. Purified or partially purified β-glucan may serve as a control in the SLP test.

The assay methods are intended for use with biological samples, such as saliva, blood, serum, cerebral spinal fluid, cervicovaginal samples, for example. Other biological samples, such as food samples, which are tested for contamination, such as by bacteria or insects, also are contemplated. Target analytes include, but are not limited to: nucleic acids, proteins, peptides, and antigens or antibodies indicative of bacterial, which may be adapted for colorimetric assays.

### IV. Assay Kits

The assay methods described in Section “III” above may be provided in a BacTx® kit (described in pending U.S. application Ser. No. 13/898,683, and hereby incorporated by reference). The kit may consist of lyophilized peptidoglycan detection reagents, sample preparation reagents, controls and microfuge tubes. The kit for detecting peptidoglycan or β-glucan in a sample may comprise a prophenoloxidase cascade system, a phenoloxidase substrate that generates a quinone reaction product, and 3-methyl-2-benzothiazolinone hydrazone or derivative thereof. The prophenoloxidase cascade system is obtained from insect plasma or hemolymph, or obtained from silkworm larvae plasma. The prophenoloxidase cascade system used in the kit comprises prophenoloxidase activating enzyme, prophenoloxidase, and a serine proteinase cascade. The prophenoloxidase cascade system may further comprise a peptidoglycan binding protein or a β-glucan binding protein. Still further the kit comprises a phenoloxidase substrate that generates a quinone reaction product. The phenoloxidase substrate that generates a quinone reaction product may be L-3,4-dihydroxyphenylalanine (DOPA), dopamine, or other mono- or di-phenol compound.

The kit may further comprise a peptidoglycan standard, wherein the peptidoglycan standard is isolated bacterial peptidoglycan, whole bacterial extract, or inactivated whole bacteria. The kit may further comprise a β-glucan standard, wherein the β-glucan standard is isolated fungal β-glucan, whole fungal extract, or inactivated whole fungi. The kit may comprise an extraction solution. The extraction solution may be an alkaline extraction solution. The kit may also comprise a neutralization buffer. Alternatively, the kit may provide 3-methyl-2-benzothizolinone or derivative thereof dissolved in a neutralization buffer. In another alternative, the kit may further comprise a dry detection reagent containing MBTH or derivative thereof co-lyophilized with a prophenoloxidase cascade system and a phenoloxidase substrate that generates a quinone reaction product. The kit may still further comprise instructions for use with the optical reader apparatus and software. Reagents in the kit may be provided in individual containers or as mixtures of two or more reagents in a single container. Any of the reagents may be provided as a liquid or as a dry powder (e.g., lyophilized).

As used herein, the BacTx® kit consists of three liquid reagents for sample preparation (Lysis, Extraction, and Neutralization Reagents), disposable microcentrifuge tubes, positive and negative controls, and multiple, single-use BacTx® Reaction Tubes. The BacTx® kit is to be used in conjunction with the assay device described in section “V” of the present inventions.

### V. Assay Device

The assay device described herein comprises (1) an optical reader apparatus, (2) an optical reader apparatus software, and (3) barcode scanner utilizing the assay methods set forth in section “III”. The optical reader apparatus may be referred to herein as the “BacTx® reader”. The optical reader apparatus software may be referred to herein as the “BacTx® reader software”. The assay device is described below.

i. Optical Reader Apparatus, Software, and Scanner

The BacTx® reader is a bench-top laboratory instrument intended for use by technicians trained in the BacTx® procedure and who follow all instructions indicated in the package insert. Laboratory technicians manually perform sample processing which includes (1) sampling platelet units; (2) separating cellular membranes from supernatant; and (3) hydrolysis of membranes to liberate peptidoglycan. After sample processing is complete technicians transfer the indicated sample volume into BacTx® reaction tubes. The tubes are mixed and then placed into one of the vacant detection wells in the instrument. The instrument monitors all reaction tubes and indicates positive or negative results based on predefined rules.

Producibility—Fabricated components should be carefully evaluated for features, fabrication methods, and tolerances, to maximize component producibility.

Assembly—Where possible, the design should use top down assembly and adequate space for cables, user maintenance, and service personnel.

Modularity—Where possible, the design should use modular assemblies for manufacturing and service. The instrument should be designed so that removal of no more than one assembly is required to access any assembly.

Testability—The design should include built-in-test, embedded software utilities for test, and test points on PCBs to verify instrument operational status after user or preventive maintenance, or unscheduled service.

Reliability—Where possible, the design should minimize the number of active components, use high quality components, and de-rated components for long life.

Obvious symmetry—Assemblies and components that are similar in appearance should incorporate features of asymmetry in order to prevent the incorrect assembly, repair, or operation of the instrument.

Stability of assemblies—Instrument assemblies and subassemblies should contain integral features to prevent any damage when placed on a flat surface. This applies to assemblies that are accessible or removable by authorized service personnel.

System Requirements

The following requirements apply to the BacTx® reader.

Environment

The following environmental requirements apply to all components of the BacTx® reader, unless otherwise specified. If the Consumables impose narrower limits, additional or modified requirements will need to be developed.

Operating Conditions

The BacTx® reader shall operate between room temperatures of 19° C. and 26° C. The BacTx® reader shall operate between 20% and 80% relative humidity, non-condensing. The BacTx® reader shall operate between 0 to 2240 m (0 to 7,350 feet).

Storage & Transportation Conditions

The BacTx® reader shall have a storage and transportation temperature range of −15° C. to 65° C. while packaged. This excludes the Consumables. The BacTx® reader shall have a storage and transportation relative humidity range between 10% and 90%, non-condensing while packaged. The BacTx® reader shall have a storage altitude range between 0 to 2240 m (0 to 7,350 feet) while packaged. Note: this is based on the altitude of Mexico City. The BacTx® reader shall have a transportation altitude range between 0 to 6096 m (0 to 20,000 feet) at standard barometric pressure while packaged. Note: this is based on ground, ship, and air transportation.

Power

The BacTx® reader shall operate when powered with AC sources of 90-264 VAC at 47-63 Hz. The BacTx® reader shall consume no more than 94 Watts during steady state operation. Note: steady state is the average of power during the final 15 minutes processing fully populated tubes. The BacTx® reader shall be designed to utilize standard NEMA power cord connectors: Note: This is to facilitate configuration for worldwide markets. The power cord on the BacTx® reader shall be located in a position that precludes interference with instruments that are adjacent to its sides.

Fluid Spills and Breakage

The BacTx® reader shall contain the fluid spill within the instrument from one REACTION TUBE. The BacTx® reader shall be designed to facilitate removal of a broken reaction tube.

Acoustic Noise

During normal operation sound emitted from the system shall be not more than 65 dB (A) when measured 1 meter in front of the system/edge of bench at Instrument base height.

Physical Configurations

The BacTx® reader shall operate on a bench top. The BacTx® reader shall be a self-contained unit that is not expandable.

Dimensions

The BacTx® reader depth shall not exceed 18″ excluding the external power cord and any airflow/switch clearances. The BacTx® reader width shall not exceed 15″ excluding the external power cord and any airflow/switch clearances. The BacTx® reader height shall not exceed 20″.

Weight

The BacTx® reader shall weigh no more than 15 lbs.

Materials

The external surfaces of the BacTx® reader shall withstand wipe-down with cleaning solutions without change to visual or structural properties.

Installation

The BacTx® reader shall be capable of being installed by users.

System Performance

The BacTx® reader shall process up to 8 REACTION TUBEs simultaneously. The BacTx® reader shall process individual REACTION TUBEs asynchronously.

External Communications The BacTx® reader shall be capable of exporting data via a USB2 type A port. The BacTx® reader shall be in US English. The BacTx® reader hardware shall be capable of supporting a LIS. The BacTx® reader shall incorporate an Ethernet port (8P8C connector). The BacTx® reader shall be support an external bar code reader via a USB2 type A port. The BacTx® reader barcode reader shall read 1D and 2D barcodes.

The BacTx® reader barcode reader shall be capable of reading the ISBT barcodes on platelet bags. The BacTx® reader barcode reader shall be capable of reading Code 128 barcode symbology. Note: This is the barcode symbology used to print ISBT labels.

The BacTx® reader barcode reader shall be capable of reading the BACTX® ID label barcodes.

Safety & EMC

Instrument shall prevent user exposure under normal operating conditions to broken REACTION TUBEs.

User Maintenance & Decontamination

The BacTx® reader is intended to require minimal User maintenance, except for periodic cleaning and/or decontamination. The BacTx® reader shall withstand decontamination by wipe-down with cleaning solutions without damage.

Installation & Service

The BacTx® reader shall accept software upgrades via the USB port. The BacTx® reader shall be serviced by shipment to the manufacturer.

Reliability

The BacTx® reader will have a predicted MTBF of 24 months at release.

Manufacturing

The BacTx® reader shall be designed to incorporate modular testing and assembly.

Performance Requirements

Thermal Requirements

The BacTx® reader shall limit the thermal gradient between any two REACTION WELLs to less than 2° C. The BacTx® reader shall limit the thermal difference between the specified operational room temperature and the temperature at REACTION WELLS to less than 4° C.

Mechanical Requirements

The BacTx® reader shall allow random access loading of REACTION TUBEs. The BacTx® reader shall allow random access removal of REACTION TUBEs. The BacTx® reader shall provide a means to incubate a REACTION TUBE without mixing for the initial 15 minutes×10 seconds of an assay. The BacTx® reader shall provide a means to mix a REACTION TUBE for the final 15 minutes×10 seconds of an assay. The BacTx® reader shall mix the contents of the REACTION TUBEs for a 15×0.25 second interval of each minute. The BacTx® reader shall mix the contents of all REACTION TUBEs in the same manner. The BacTx® reader shall monitor the speed of the REACTION TUBE mix motor. The BacTx® reader shall monitor the mix frequency of each REACTION TUBE. The REACTION WELL shall accept REACTION TUBES with affixed manufacturing labels. The REACTION WELL shall accept REACTION TUBES with affixed manufacturer's BacTx® ID LABELS. The BacTx® reader shall provide a means to cover each REACTION WELL. Note: Protects the assay from external contaminants and stray light. The BacTx® reader shall detect the state of the REACTION WELL cover.

Optical Requirements

The BacTx® reader signal drift shall be less than 0.01 absorbance units over a 30 minute time frame. Note: The highest change in absorbance observed with negative samples (n=489) was 0.16. The lowest change in absorbance observed with positive samples (n=296) 296 was 0.80. The BacTx® reader signal drift due to a temperature change of 5 degrees C. shall be less than 1 milliabsorbance units. The BacTx® reader shall exhibit a maximum within-channel coefficient of variation (n=10) of:

| | |
|---|---|
| OD 0.1 | 1.5% |
| OD 1.0 | 0.4% |

The BacTx® reader shall exhibit a maximum between-channel coefficient of variation (n=8) of:

| | |
|---|---|
| OD 0.1 | 15% |
| OD 1.0 | 15% |

The BacTx® reader as part of its CALIBRATION routine shall measure Vdark (signal with light source off) for each OPTICAL CHANNEL without a REACTION TUBE. The BacTx® reader shall measure VDetectorLedOn (signal with light source on) for each OPTICAL CHANNEL without a REACTION TUBE as part of its CALIBRATION routine. The BacTx® reader shall CALIBRATE all OPTICAL CHANNELs on power-up. The BacTx® reader shall measure Vdark and VDetectLedOn in an OPTICAL CHANNEL prior to performing an assay. The BacTx® reader shall CALIBRATE an OPTICAL CHANNEL prior to performing an assay if Vdark drifts more more than 0.5% of the 100% transmittance value from its stored CALIBRATION value. The BacTx® reader shall CALIBRATE an OPTICAL CHANNEL prior to performing an assay if VDetectLedOn drifts by more than 1.25% of the stored CALIBRATION value. The BacTx® reader shall prevent the use of an OPTICAL CHANNEL that is not CALIBRATED. The BacTx® reader shall take an INITIAL READING within 10 seconds of REACTION TUBE DETECTION. The BacTx® reader shall take READINGS no sooner than 10 seconds after mixing samples. The BacTx® reader shall take READINGS at 30 second intervals during an assay. The BacTx® reader shall take READINGS for 30 minutes on all assays or until a sample is defined as a POSITIVE. The BacTx® reader shall exhibit a linear absorbance response from 0.0 to at least 1.2 OD.

The BacTx® reader shall measure Vdark and VDetectLedOn for each OPTICAL CHANNEL as part of power on self-test. The BacTx® reader shall use an LED with an emission midpoint at 505 nm±5 nm. The BacTx® reader shall use an LED with a luminous intensity of at least 9800 millicandelas (mcd). The BacTx® reader shall use a silicon PIN photodiode. The body of the LED in each OPTICAL CHANNEL shall be centered at the midpoint of a 300 microliter volume in the REACTION TUBE.

Electronic Module Requirements

The BacTx® reader shall terminate an assay if the initial absorbance reading is greater than the maximum absorbance that is linear minus (600 milliabsorance units+(3×between channel-to-channel standard deviation at maximum absorbance)). Note: The above value is driven by the need to ensure adequate dynamic range to detect a minimum absorbance change of 0.5. The BacTx® reader shall void a current assay if any ASSAY PARAMETERS are outside specified limits. The BacTx® reader shall void a current assay if any OPERATIONAL PARAMETERS are outside specified limits. The BacTx® reader shall prevent further use of an OPTICAL CHANNEL if any OPERATIONAL PARAMETERS are outside specified limits.

Note: Example—prevent the use of an OPTICAL CHANNEL if the initial reading demonstrates an absorbance value that is greater than the CALIBRATION VLedOn value. The BacTx® reader shall use a single LED and paired sensor per OPTICAL CHANNEL. The BacTx® reader shall have a means to track ASSAY TIMING to a maximum of 0.1 second resolution. The BacTx® reader shall save all assay data collected. The BacTx® reader shall not allow Technicians to delete any saved assay data.

System Level Requirements

The BacTx® reader shall be capable of continuously processing REACTION TUBEs. The BacTx® reader shall initiate processing of a REACTION TUBE only after a sample has been identified via a BACTX® SAMPLE ID label or manual input. The BacTx® reader shall provide functionality to require REAGENT KIT LOT NUMBER to be input before processing a REACTION TUBE. The BacTx® reader shall perform POSITIVE REACTION TUBE DETECTION. The BacTx® reader shall start assay timing (T=0) once a REACTION TUBE is detected in a REACTION WELL. The BacTx® reader shall emit an audible ALERT 60 seconds after the START ASSAY GUI FIELD is triggered if a REACTION TUBE has not been detected in a REACTION WELL. The BacTx® reader shall VOID an assay 120 seconds after the START ASSAY GUI FIELD is triggered if a REACTION TUBE has not been detected in a REACTION WELL. The BacTx® reader shall tag an assay result if the REACTION WELL COVER is not closed within 5 minutes of REACTION TUBE DETECTION. The BacTx® reader shall stale-date the BACTX® SAMPLE ID if the assay is not completed within 24 hours. The BacTx® reader shall not process a REACTION TUBE with stale-dated BACTX® SAMPLE ID. The BacTx® reader shall emit an ALERT when a REACTION TUBE is removed during processing. The BacTx® reader shall void an assay if a REACTION TUBE is removed during processing. The BacTx® reader shall void the BACTX® SAMPLE ID when an assay is voided. The BacTx® reader shall not process a REACTION TUBE with a voided BACTX® SAMPLE ID. The BacTx® reader shall provide functionality to read the BACTX® SAMPLE ID label. The BacTx® reader shall be able to process CONTROL samples simultaneously with platelet SAMPLES. The BacTx® reader shall allow users to enter the ISBT PRODUCT CODE barcode ID.

User Interface Requirements

The BacTx® reader shall have a touch screen that can be used with gloved hands. The BacTx® reader provides functionality to control system workflow.

GUI Requirements

The BacTx® reader GUI shall require a user to be logged in before initiating sample processing. The BacTx® reader GUI shall support switching between user accounts (logging out/logging in) without interrupting processing. The BacTx® reader GUI access control functionality shall provide the following levels of access: Factory Lab Manager/Supervisor Technician. The BacTx® reader GUI shall support multiple accounts at the Technician and Supervisor access level. The BacTx® reader shall provide the Supervisor rights to control the access to account setup, data fields and certain system operational parameters. The BacTx® reader GUI shall limit access to stored data by access level. The BacTx® reader GUI shall store all user credential information and account login/logout time stamps. The BacTx® reader GUI shall limit user login to 1 user at a time. The BacTx® reader GUI shall ALERT user if ASSAY PARAMETERS are outside specified limits. The BacTx® reader GUI shall ALERT the user if any OPERATIONAL PARAMETERS are outside specified limits. The BacTx® reader GUI shall provide functionality to display status information. The BacTx® reader GUI shall provide functionality to display the amount of time left in an ASSAY. The BacTx® reader GUI shall provide functionality to display the elapsed time for an ASSAY. The BacTx® reader GUI shall provide functionality to allow a USER to display processed SAMPLEs. The BacTx® reader shall provide functionality to display a message when USER attention is required. The BacTx® reader shall provide the capability for a SUPERVISOR to configure reports.

Usability Requirements

The BacTx® reader shall allow users to log out of the BacTx® reader while a SAMPLE is processing without affecting the processing of the SAMPLE. The BacTx® reader shall allow users to log into the BacTx® reader while samples are processing without affecting the processing of the SAMPLEs. The BacTx® reader shall have audible alarms to indicate when immediate attention is required. The BacTx® reader shall be user friendly. As a goal the BacTx® reader shall be capable of generating a WORKLIST from ISBT barcodes on platelet units and BACTX® SAMPLE ID labels. The BacTx® reader shall be designed to be operated by a user with gloved hands.

Consumables

Manufacturer will specify all other Consumables in other documents that will contain explicit interface requirements. The BacTx® reader shall be designed to hold Manufacturer REACTION TUBEs which have an outer diameter of 8.00 to 8.30 mm and are 43.5×1 mm tall (Pheonix Glass, LLC part number PX-157; Apr. 13, 2009). The BacTx® reader shall utilize BACTX® SAMPLE barcoded ID labels.

The software and functionality requirements, design, and the Software Verification and Validation activity are set forth below for the BacTx® reader (BacTX® reader.) The software is referred to herein as the “BacTx® reader software”. The BacTx® reader software has the following features and functions: 1) internationalized GUI interface; 2) multiple language support (English for first revision); 3) automatic calibration of instrument specific parameters; 4) accepting and responding to USER input via a barcode scanner and/or a touch screen; 5) user Accounts and access levels for a) Laboratory Technician, b) Lab Supervisor/Manager, c) Factory/Engineering, d) Account logout after a configurable number of minutes; 6) guide and manage assay workflow to correlate Donor Identification number (DIN) and Product Information Code (PIC) to the BacTx® ID and sample to Reagent Lot Number; 7) transparent instrument calibration to inform the USER only on a failure and to log calibration of date and time; 8) assay processing wherein a) assays can be performed in a random access mode, b) control and monitor agitation, c) monitor tube insertion/removal, d) monitor cap positions, e) user indicators, f) obtain measurements; 9) assay types such as a) controlled which requires USER to input IDs, b) Stat which is immediate processing and limited USER Id inputs, c) duplicate: split sample into two One Platelet Id and Two BacTx® Ids, d) QC: Positive and negative control samples; 10) process acquired data, display results, and store results; 11) gives a clear and final result of PASS or FAIL for all samples analyzed by the instrument; 12) respond to error conditions and alerting the USER via visual and audio cues; 13) software upgradable by supervisory and/or factory access; 14) work list: current assays in progress; 15) history of assays moved from work list to archive; 16) reports to select and export results to file or select and export log to file; 17) export data to move/copy log files or results to USB device or move/copy results; 18) software upgrade; 19) miscellaneous functions to set time, disable the audio part of an ALERT; and 20) self-test to check optical and mechanical components for proper operation, disable assay functionality on failure, and re-enable assay functionality on pass.

System Description

The BacTx® reader is designed to perform a single-wavelength colorimetric cutoff assay, for the detection of bacteria in platelet samples. Other modification can be made to the BacTx® reader to accommodate detection of contaminants, bacterial, fungal, pathogens, or analytes in a sample.

Systems Hardware Architecture

The BacTx® reader software controls the instrument. The hardware consists of two items, the Single Board Computer (SBC) and the Interface Board Controller (IBC). The SBC communicates to the IBC via a serial communications interface. User inputs of the instrument are obtained through a touch screen interface, a barcode reader, and a USB memory stick. User output will be displayed to the touch screen display. Files will be written to a USB memory stick for exporting data. Internal operational data and parameters will be store as records on the SBC.

The main function of the instrument is to process an assay. The instrument can process up to eight samples simultaneously in a random access format. The application software running on the SBC is responsible for controlling and obtaining data from the hardware devices, which are connected to the IBC, associated with processing. The SBC treats the IBC as an integrated device.

The following devices interface to the IBC:
Sensors used to monitor the state of each REACTION WELL cover.
Controls used to engage, independently, agitation for each REACTION TUBE.
Control used to engage the REACTION TUBE mix actuator.
Sensors used to monitor the speed of the REACTION TUBE mix actuator.
Sensors used to monitor the agitation speed of each REACTION TUBE.
Sensors used to monitor the presence of a REACTION TUBE in a REACTION WELL.
Controls used to engage the optical transmitters.
Sensors used to obtain the dark and light optical density measurements.
Indicators used to indicate the status of a REACTION WELL or to prompt an action associated with that REACTION WELL to the USER.

System Environmental Constraints

The system uses a configuration entity internally, known as the “Device Calibration Record” for storing calibration information. This is a dynamic table which must be managed in a way that enables a non-privileged user to perform the calibration procedures. However, this record is also protected by the system from unauthorized modifications; the record is saved as “read only”.

User Characteristics

The BacTx® reader system is assessable by two levels of USERS who are the intended operators of the BacTx® reader: Lab Supervisor/Manager and trained Laboratory Technicians. Lab Technicians are considered non privileged users. A third level of access exists, the FACTORY user who is a privileged user, which is reserved for engineering and diagnostic purposes.

Software Requirements

In this section the term software or firmware will be synonymous with the FCB firmware application.

Notations

Throughout this document, certain terms are synonymous. Tubes refer to the REACTION TUBES that are placed in a REACTION WELL channel. Light readings refer to the VDetectLedOn value; optical signal received with light source on. Dark readings refer to the Vdark value; optical signal received with light source off. The term LAB USER refers to LAB SUPERVISOR/MANAGER and the LAB TECHNICIAN.

The term software refers to the application software running on the SBC. The term firmware refers to the embedded controller code running on the IBC.

General Requirements

The software shall use English as the default Language. The software shall be coded with provisions to display text to the screen in languages other than English.

Software Initialization

Initialization is the period entered immediately after a reset. Upon initialization, the software application running on the SBC needs to successfully communicate with the IBC and insure its proper connectivity. There are two conditions to be met for the software to proceed beyond this stage: communications must be established to the IBC and the REACTION TUBE MIX actuator, located off the IBC, must be tested for proper operation.
Demonstrate communications to the IBC
Test the REACTION TUBE mix actuator for proper operation
Test each REACTION TUBE WELL channel for proper optical and mechanical operation

The SBC connects to the IBC via a hardline serial port connection; hence, no USB discovery is required. However, since the connection is a single point of failure, connectivity must be established before any commands/responses can be issued to /from the IBC.

The software shall detect the operation of the Interface Board Controller (IBC) by initiating communications with it. The software shall alert the user if there was a communications failure with the IBC. The software shall proceed with initialization once communication has been established with the IBC. The software shall ALERT the user if communication cannot be established with the IBC. The software shall disable all assay functions if communication cannot be established with the IBC. The software shall confirm the operation of the REACTION TUBE mix actuator by enabling agitation for a minimum of TBD seconds.

The software shall ALERT the USER of the REACTION TUBE mix actuator operational status if not operating properly. The software shall proceed with initialization if the REACTION TUBE mix actuator is operating properly. The software shall disable the REACTION TUBE mix actuator if not operating properly. The software shall disable all assay functions if the REACTION TUBE mix actuator is not operating properly. The software shall confirm the mechanical operation of each REACTION TUBE WELL channel. The software shall confirm the optical operation of each REACTION TUBE WELL. The software shall ALERT the USER of a REACTION TUBE WELL channel operational status if not operating properly.

The software shall proceed with initialization if a REACTION TUBE WELL channel is operating properly. The software shall disable each REACTION TUBE WELL channel that is not operating properly. The software shall disable each REACTION TUBE WELL channel if a REACTION TUBE is present. The software shall tag the results field of the results record as aborted if a REACTION TUBE is present in the REACTION TUBE WELL channel. (Note: during instrument initialization only.) The software shall provide a mechanism for the LAB SUPERVISOR/MANAGER to reinitialize a REACTION TUBE WELL channel. The software shall tag the results field of the results record as aborted if an assay was running when the instrument experienced a power loss. The software shall contain a reason field in the results record to indicate the cause of an aborted assay. The software shall not allow any LAB USER to force a REACTION TUBE WELL channel to be enabled if previously disabled.

Instrument Calibration

In order for the software to determine the correct absorbance readings, it needs to have some reference points for the signals it reads in the instrument. These reference points are used for calibration and are stored in a calibration record. The record containing the calibration is created automatically by the system when the user first operates the reader. The calibration record contains the instruments calibrated dark and light readings.

The device configuration record contains:
The dark and light range values used to compare with the measured readings.
The date and time of the last calibration

Since the functionality of the instrument is integrated, the SBC is hard wired to the IBC. There is no need to correlate the unique serial number to a calibration record.

During initialization, the software shall run a new calibration if a calibration record doesn't exist in the system. Software shall prevent the use of an OPTICAL CHANNEL when in calibration mode. Software shall prevent the use of an OPTICAL CHANNEL that is not calibrated. The software shall instruct the USER to remove tubes from the REACTION WELL channels prior to running the calibration. The software shall instruct the USER to cover the REACTION WELL channels prior to running calibration. The software shall ALERT the USER that the REACTION WELL channel is not enabled if the channel fails calibration. The software shall disable the REACTION WELL channel that failed calibration. The software shall store the calibration values in the calibration record at the time calibration is performed. The software shall store the calibration date/time that the calibration was performed. The software shall disable any OPTICAL CHANNEL that is not within the range of the calibration values.

Optical Channel Initialization

Upon successfully loading the calibration information for the reader device or a successful calibration, the software initializes the OPTICAL CHANNELs.

The software shall take and store a dark reading for each OPTICAL CHANNEL. The software shall take and store a light reading for each OPTICAL CHANNEL. The software shall compare the OPTICAL CHANNEL's dark reading to the stored calibration dark reading value. The software shall compare the OPTICAL CHANNEL's light reading to the stored calibration light reading value.

Data Connection (Serial Connection)

The IBC and the SBC are connected internally within the instrument via a serial port. The data connection between them must be continuous.

The software shall monitor the connection between the SBC and the IBC every 60 seconds±1 second. The software shall ALERT the LAB USER when communication is lost. The software shall ALERT the LAB USER when communication is restored, if previously lost. The software shall prevent the LAB USER from running new assays if communication is lost. The software shall abort running assays when communication is lost. The software shall log communication connection transitions.

USER Input

The system LAB USER is expected to load sample tubes, enter or scan sample identification, initiate the assay, and respond to GUI specific prompts. Assay data acquisition and timing are entirely controlled by the software.

User Interface Requirements

The software shall be capable of reading Donor Identification Number (DIN) barcodes using the bar code reader. The software shall be capable of entering DIN barcodes using the touch screen. The software shall be capable of reading BacTx® IDs using the bar code reader. The software shall be capable of entering the BacTx® IDs barcode ID using the touch screen. The software shall be capable of reading REAGENT LOT NUMBER using the bar code reader. The software shall be capable of entering the REAGENT LOT NUMBER barcode ID using the touch screen. The software shall be capable of reading PRODUCT INFORMATION CODE (PIC) using the bar code reader. The software shall be capable of entering the PRODUCT INFORMATION CODE barcode ID using the touch screen.

Access Control

The software shall support the ability for USERS to log in and out of the instrument. The software access control functionality shall provide LAB SUPERVISOR/MANAGER level of access. The software access control functionality shall provide LAB TECHNICIAN level of access. The software access control functionality shall provide a FACTORY level of access. The software shall support multiple USER accounts at each level of access. The software shall logout the USER after a configurable number of minutes of no activity. The software shall provide a method to configure the USER account inactivity logout time on a per user basis. The software shall support the ability for LAB SUPERVISOR/MANAGER USERS to add LAB SUPERVISOR/MANAGER accounts. The software shall support the ability for LAB SUPERVISOR/MANAGER USERS to add LAB TECHNICIAN accounts. The software shall support the ability for LAB SUPERVISOR/MANAGER USERS to delete LAB SUPERVISOR/MANAGER accounts. The software shall support the ability for LAB SUPERVISOR/MANAGER USERS to delete LAB TECHNICIAN accounts. The software shall support the ability for LAB SUPERVISOR/MANAGER USERS to modify LAB SUPERVISOR/MANAGER accounts. The software shall support the ability for LAB SUPERVISOR/MANAGER USERS to modify LAB TECHNICIAN accounts. The software shall log all user credential information including account login/logout time stamps. The software shall only initiate sample processing when a user is logged in. The software shall support switching between user accounts (logging out/logging in) without interrupting processing. The software shall allow a USER to log out of the Instrument while a SAMPLE is processing without affecting the processing of the SAMPLE. The software shall allow USER to log into the BacTx® reader while samples are processing without affecting the processing of the SAMPLEs.

Usability Requirements

ISBT, BacTx® ID or the Reagent Lot ID can be barcode scanned or entered via the touch screen. An ALERT has an audio and a user prompt component.

The software shall support audio alarms to indicate when immediate attention is required by the USER. The software shall provide functionality to display a message when USER attention is required by the USER. The software shall provide functionality to display the amount of time remaining in an ASSAY. The software shall provide functionality to allow a USER to search for processed SAMPLEs. The software shall provide functionality to display a message when a CALIBRATION is required. The software shall write the assay result metrics to a stored record. The software shall clear the assay result metrics from the display when storing a record. The software shall support an option in the LAB SUPERVISOR/MANAGER access account to require a supervisor's review to write assay result data to a stored record. The software shall indicate to the USER in which REACTION WELL to insert the REACTION TUBE. The software shall ALERT the USER when an assay has failed. The software shall allow the USER to turn off the audio portion of the ALERT. The software shall allow the USER to process a STAT assay which requires no DIN, PIC, or BacTx® Id. The software shall associate a DIN to the PIC by entering the PIC followed immediately by the BacTx® Id. The software shall associate a PIC with the BacTx® ID by entering the PIC followed immediately by the BacTx® ID. The software shall require the BacTx® ID to be associated with the DIN prior to insertion of the REACTION TUBE into the REACTION WELL. The software shall prompt the USER to enter the BacTx® ID prior to insertion of the REACTION TUBE into the REACTION WELL. The software shall provide functionality to set the date/time of the system. The software shall not allow the USER to enable the Daylight Savings Time operating systems auto update setting. The software shall not allow the USER to change the system time if any assays are running.

ATP Test Software

The ATP software shall provide functionality to perform absorbance readings in all optical channels. The ATP software shall provide functionality to activate the motor and report back rpm versus time. The ATP software shall provide the functionality to individually activate solenoids according to a predetermined or random sequence. The ATP software shall provide the functionality to individually oscillate REACTION WELLS according to a predetermined or random sequence. The ATP software shall provide the functionality to report the oscillation frequency for REACTION WELLS. The ATP software shall provide functionality to export data files. The ATP software shall provide the functionality to export a time stamp of all events required to complete an assay. The ATP software shall provide the functionality to export a time stamp of all events required to complete an assay. The ATP software shall provide the functionality to run CONTROL assays.

Agency Testing Software

The application software shall provide a function to run the system continuously during agency testing. The Agency Testing Software (ATS) application software shall provide functionality to read and mix up to 8 assays simultaneously.

The optical reader apparatus and software are integrated into a single unit. The unit may be interconnected via any suitable means including over a network, e.g. to another processor or computing device. The data export means may take the form of a portable processing device that may be carried by an individual user e.g. lap top, and data can be transmitted to or received from any device, such as for example, server, laptop, desktop, PDA, cell phone capable of receiving data, and the like. A wireless device can be used to receive data and forward it to another processor over a telecommunications network, for example, a text or multi-media message, or a medical hospital, patient record network, medical database.

The data may be sent or distributed among a plurality of processors, which may be interconnected over a network. Further, the information can be encoded using encryption methods, e.g. SSL, prior to transmitting over a network or remote user. The information required for decoding the captured encoded images taken from test objects may be stored in databases that are accessible to various users over the same or a different network.

The data is saved to a data storage device and can be accessed through a web site. Authorized users can log onto the web site, upload test data, and immediately receive results on their browser. Results can also be stored in a database for future, reviews.

The web-based service may be implemented using standards for interface and data representation, such as SOAP and XML, to enable third parties to connect their information services and software to the data. This approach would enable seamless data request/response flow among diverse platforms and software applications.

The test data may be shared with medical hospital, patient record network, medical database, testing facilities, quality control organizations and the like, which are then access means by third parties authorized at such facilities and organizations.

### VI. Assay Detection System

The assay detection system described herein comprises a (1) kit set forth in section “IV” and (2) an optical reader apparatus and software set forth in section “V” utilizing the assay methods set forth in section “III”.

Example 1 provides stepwise instructions of the assay detection system utilizing, by way of example, the BacTx® Kit and optical reader apparatus and software of the present invention. Example 1 details the various graphical user interface features for each step of the sample analyses from sample processing, bar code scanning, sample agitation, testing of positive and negative controls, testing of samples, to test data results, and data storage and export capabilities.

Example 2 further breakdowns in a chart-wise fashion the pre-processing and processing steps of the sample using the assay detection system of the present invention. The assay formats include manual processing, full-logged processing, partial log processing, STAT processing, and Retest scenarios.

The assay detection system detects the formation of a bright red reaction product which is the end-product of an enzyme cascade (a sequence of enzyme reactions) described in section “III”. In the absence of bacteria the serine protease cascade is inactive; in the presence of a biological polymer common to all bacteria (peptidoglycan) serine proteases in the reagent are activated. Activated serine proteases ultimately oxidize dopamine to dopaquinone which forms the bright red reaction product when it combines with a chemical indicator.

The assay detection method is distinct from clinical chemistry assays and immunoassays since it is based upon triggering an enzyme cascade. Once the cascade is activated, the concentration of active enzymes moves to saturation independent of the input concentration of the target (bacterially derived peptidoglycan). Thus, once the threshold is reached the final absorbance is not proportional to initial target concentration. That is, REACTION TUBEs with a high concentration of bacteria and REACTION TUBEs with a low bacterial load (>the lower limit of detection) can yield roughly equivalent signal output. The assay method is formulated such that a change of 0.5 absorbance units in 30 minutes indicates the presence of bacteria.

Assay Processing

Assays are conducted in glass tubes (although plastic tubes or plastic microtiter plates may be used); an eight-position reader measures optical signal independently for each REACTION TUBE, and transmits the signal to the SBC from the IBC. The assay's chemical biological reaction will cause an increase in absorbance of the monitored signal. This signal is monitored for a specified period of time.

The USER will enable an assay via a control in the GUI. The software will indicate to the USER which REACTION WELL channel to put the sample into. The USER will have 120 seconds to place the sample into the REACTION WELL channel before the assay is aborted. The USER will be ALERTed after 60 seconds to place the sample into the REACTION WELL channel if not done. The assay start time (T=0) is when the REACTION WELL TUBE is detected in the REACTION WELL channel. The first absorbance reading is taken after the REACTION WELL TUBE is detected in the REACTION WELL channel. The USER is ALERTed after 5 seconds of T=0 if the cover is open. The ALERT is removed by the software when the cover is closed. The software tags a field in assay result record if the cover is not closed within 5 minutes of T=0. The software aborts the assay if a REACTION WELL TUBE is removed before assay conclusion. Absorbance readings are taken at 30 second intervals starting from the first reading. Absorbance readings can never occur during agitation and within 10 seconds of agitating.

The software shall be capable of processing individual reaction tubes asynchronously. (random access) The software shall be capable of processing eight reaction tubes simultaneously The software shall confirm the REACTION TUBE mix actuator speed prior to engaging the REACTION TUBE Well channel to start the assay agitation. The software shall measure REACTION TUBE mix actuator speed at a minimum interval of 60 seconds. The software shall ALERT the USER at a minimum 5 second intervals if the cover is opened while the assay is running. The BacTx® reader shall terminate an assay if the initial absorbance reading is greater than the maximum absorbance that is linear minus (600 milliabsorance units+(3×between channel-to-channel standard deviation at maximum absorbance)). The software shall abort a current assay if any OPERATIONAL PARAMETERS are outside specified limits. The software shall disable an OPTICAL CHANNEL if any OPERATIONAL PARAMETERS are outside specified limits. Note: Example—prevent the use of an OPTICAL CHANNEL if the initial reading demonstrates an absorbance value that is greater than the CALIBRATION light reading value. The software shall have a means to track ASSAY TIMING to a maximum of 0.1 second resolution. The software shall be capable of continuously processing REACTION TUBEs. The software shall initiate processing of a REACTION TUBE only after a BACTX® SAMPLE ID label has been inputted. The software shall make inputting a BACTX® SAMPLE ID label prior to starting an assay as a configurable in the LAB SUPERVISOR/MANAGER account access. The software shall initiate processing of REACTION TUBEs only after a REAGENT KIT LOT NUMBER has been input. The software shall perform POSITIVE REACTION TUBE DETECTION. The software shall stale-date the BACTX® SAMPLE ID if the assay is not completed within 24 hours. The software shall not process a REACTION TUBE with stale-dated BACTX® SAMPLE ID. The software shall ALERT when a REACTION TUBE is removed during processing. The software shall void an assay if a REACTION TUBE is removed during processing. The software shall void the BACTX® SAMPLE ID when an assay is aborted. The software shall not process a REACTION TUBE with an aborted BACTX® SAMPLE ID. The software shall read an ISBT bar code from the BACTX® SAMPLE ID label. The software shall be capable of reading Code 128 barcode symbology. Note: This is the barcode symbology used to print ISBT labels. The software shall be capable of reading the BacTx® ID label barcodes in Data Matrix Format. The software shall write a reading to a record when a reading has been taken. The software shall take absorbance readings at a minimum of 14 seconds after stopping agitation. The software shall tag the state of the cover as open or closed in a field of the reading record after taking a reading. The software shall ALERT the USER at a minimum of 60 seconds after the USER starts the assay if a REACTION TUBE has not been inserted into the indicated REACTION WELL. The software shall abort an assay at 120 seconds±1 second after the USER starts the assay if a REACTION TUBE has not been detected in a REACTION WELL. The software shall tag a field an assay result record if the REACTION WELL COVER is not closed within 5 minutes±1 second of detecting the REACTION TUBE in the REACTION WELL.

Control Assay Processing

The software shall be able to process CONTROL samples simultaneously with platelet SAMPLES.

Stat Assay Processing

The software shall initiate processing of a STAT sample only after the SAMPLE ID label has been inputted.

Command Interlocks

Certain actions which could result in inconsistent or erroneous results shall be locked out. The software shall prevent setting the date/time after any assay has been started and before data from that assay have been saved. The software shall prevent aborting an assay after completion and prior to storing the results. The software shall set the completion date/time of the assay as the timestamp when storing the results.

Platelet Sample Agitation

In order to maintain the desired sample consistency, the platelet samples in the REACTION TUBES require periodic agitation during the assay. Agitation of the samples is accomplished by activating the REACTION TUBE mix actuator and engaging the REACTION TUBE WELL channels which then causes the sample tube to agitate. The assay starts, T=0, when a REACTION TUBE is detected in a REACTION WELL. The sample is then incubated for 15 minutes with no agitation; this is the incubation period. At T=15 minutes mixing (agitating) is performed every minute for 15 seconds until T=30 minutes or a positive absorbance reading. The total assay time could take up to 30 minutes. Readings are taken every 30 seconds. An assay can conclude with a positive result during the incubation period. The agitation parameter values of 15 minutes, 15 seconds and 1 minute cannot be changed by any LAB USER. The RPM of the REACTION TUBE mix actuator is monitored. If the RPM is determined to be out of specification, all running assays are aborted and REACTION WELL functionality is disabled. When agitating a sample, the REACTION TUBE channel is checked for proper operation. If agitation is determined to not be operating properly, the assay is aborted and the channel will be disabled.

The software shall agitate the platelet sample(s) periodically during the assay in order to maintain the desired sample consistency. The software shall start the REACTION TUBE mix actuator when there is at least one REACTION TUBE in a REACTION WELL. The software shall stop the REACTION TUBE mix actuator when there are no REACTION TUBES in the REACTION WELLs. The software shall agitate a sample by engaging the REACTION TUBE mix actuator to a REACTION WELL channel. The software shall start the assay time (T=0) when the REACTION TUBE is detected in the REACTION WELL. The software shall incubate a REACTION TUBE starting from the assay start time T=0 for 15 minutes±0.25 second.

The software shall start the first agitation cycle of a REACTION TUBE at T=15 minutes±0.250 seconds for 15 minutes starting from the assay start time. The software shall agitate a sample for duration of 15 seconds±0.5 seconds and then not agitate for 45 seconds±0.5 seconds at a period of 60 seconds±0.5 seconds for up to 30 minutes. The software shall perform a maximum of 15 agitation cycles. The software shall perform agitate cycles until a positive absorbance reading. The software shall not provide any facilities to allow the LAB USER to modify the agitation duration. The software shall not provide any facilities to allow the LAB USER to modify the agitation period. The software shall monitor the RPM of the REACTION TUBE mix actuator every 60 seconds±1 second. The software shall abort all running assays if the REACTION TUBE mix actuator is not agitating at a frequency of 925 RPM±25 RPM. The software shall check a REACTION TUBE for agitation at 7.5 seconds±0.5 seconds from the beginning of the agitation cycle. The software shall check a REACTION TUBE channel for no agitation at 37.5 seconds±0.5 seconds from the beginning of the agitation time. The software shall abort a running assay if a REACTION TUBE channel is not agitating when agitation is enabled. The software shall abort a running assay if a REACTION TUBE channel is agitating when it when agitation is enabled. The software shall ALERT the USER when agitation for REACTION TUBE channel is not detected when agitation is enabled. The software shall ALERT the USER if the REACTION TUBE mix actuator fails to agitate. The software shall ALERT the USER if the REACTION TUBE mix actuator agitates when agitation is disabled.

Data Processing and Results Handling

A sample which crosses the cutoff absorbance threshold (a saved parameter) is considered to be contaminated and is reported as a FAIL result.

The software shall set the sample result to FAIL when the absorbance of a sample goes above the threshold absorbance cutoff value. The software shall set the sample result to PASS if the absorbance of a sample stays below the threshold absorbance cutoff value after a maximum of 30 minutes. The software shall ALERT the USER if the result of the assay is FAIL. The software shall allow the USER to manually abort a running assay. The software shall set the sample result to ABORT when the user manually aborts an assay.

Data Storage and Processing

Assay results are saved to a result record. The records can be then saved to a file. Logging information is saved to a log record. These records can be then saved to a file. This section also states the format of the file name.

The software shall save the assay data results to a result record. The software shall write the date/time to the result record. The software shall write the BacTx® ID to the result record. The software shall write the sample test result to a result record. (Enumerated results are PASS/FAIL/ABORT) The software shall provide a default name with the current date and time as part of the name when the application prompts to write results to a file. The software shall allow the LAB USER the ability to provide a different unique name for the results file. The software shall continuously record raw data to a system log record during the operation. The purpose of this information is to aid in record-keeping. The software shall continuous log all user interactions, value readings, channel state transitions, among other general internal actions taken by the software. The software shall provide the ability to write the system log record to a file. The software shall export assay reports to an USB Memory Stick. The software shall export log files to a USB Memory Stick. The software shall capable of being installed by the LAB SUPERVISOR/MANAGER USER. The software shall capable of being installed by the FACTORY user. The software shall accept upgrades via a USB port. The IBC firmware shall be upgraded via a USB port. The SBC software shall be upgraded via a USB port.

Monitoring, Error Responses and Alarms

The application is intended to provide multiple safeguards which ensure that only valid results will be reported. The software shall ALERT the LAB USER if an invalid BacTx® ID is entered prior to inserting the REACTION TUBE into the REACTION WELL.

Unless defined otherwise, all technical and scientific terms used herein have the same meaning as is commonly understood by one of skill in the art to which this invention belongs. All patents and publications referred to herein are, unless noted otherwise, incorporated by reference in their entirety. In the event a definition in this section is not consistent with definitions elsewhere, the definition set forth in this section will control.

The invention will be further described with reference to the following non-limiting examples. It will be apparent to one skilled in the art that many modifications may be made to the embodiments described below without departing from the scope of the invention. It is to be understood that these examples are provided by way of illustration only and should not be considered limiting in any way.

### EXEMPLIFICATIONS

### Example 1

### Assay Detection System

Provided below are stepwise instructions of the assay detection system utilizing, by way of example, the BacTx® Kit and optical reader apparatus and software of the present invention. The description also provides details of the various graphical user interface features for each step of the sample analyses from sample processing, bar code scanning, sample agitation, testing of positive and negative controls, testing of samples, to test data results, and data storage and export capabilities.

These instructions for use described herein contain the necessary protocols for analyzing a test sample. In brief, to test platelets for the presence of bacterial peptidoglycan, a volume of platelets is sterilely sampled from the platelet bag and added to a microfuge tube containing Lysis Reagent and mixed. The microfuge tube is then briefly centrifuged to pellet insoluble platelet debris and bacterial cell wall fragments, if present. The peptidoglycan present at the bottom of the tube is then homogenized in Extraction Reagent. The alkaline Extraction Reagent effectively releases peptidoglycan from bacterial cell walls for optimal detection. Lastly, the suspension is added to a clean microfuge tube containing Neutralization Reagent and the tube is mixed by inversion.

From the resulting sample, an aliquot is added to a reaction tube containing lyophilized detection reagents and the tube is vortexed. The bar code on the reaction tube is scanned and the tube is then placed in the optical reader apparatus. The optical reader apparatus is a photometer which automatically monitors the detection reaction and interprets the result using software installed on the provided laptop PC. If bacteria are detected within the 30-minute reading time, a “Fail” result accompanied by an optional audible alarm is generated; otherwise, a “Pass” result will be recorded.

Document Overview

This document is written from the perspective of the user. The user may be a lab tech, lab supervisor, lab manager, scientist, testing facility personnel, factory personnel, or the like. The following lays out user interactions starting with instrument boot and power-on diagnostics. It then covers running assays and controls. Lastly, it covers administration/configuration.

Philosophy

The following philosophical points drive user interactions: data integrity is paramount, simplify user interaction, and provide the user with a consistent experience

Definitions

Informational: Information is conveyed to the user via some sort of message

Alert: User action is required now

Assay Processing

All users have access to BacTx® ID labels. All users will have access to an attached bar code reader. Platelet unit bar codes adhere to the ISBT standard.

Reagents

The primary reagent is delivered in unit doses in BacTx® Reaction Tubes (Part # CB-B005-032). Each reagent kit contains 32 sealed glass tubes; each reaction tube contains lyophilized detection reagent for a single test. Users will not mix different lots of reaction tubes. Users will not mix controls from one reagent lot with reaction tubes from another reagent lot. The control reagents are: Positive Controls (Part # CB-P012-000) which is peptidoglycan from B. subtilis in MOPS buffer; Negative Control (Part # CB-N031-000) which is MOPS buffer with gentamicin.

Sample Processing Disposables

Samples must be processed prior to transferring a sample to a reagent tube. Each kit is provided with a sufficient amount of materials to process all 32 BacTx® Reaction Tubes. This includes both (a) sterile 2 mL microcentrifuge tubes and (b) sterile 1.5 mL microcentrifuge tubes.

Bulk Reagents for Sample Workup

The user does not need to provide any information to the system for the following bulk reagents.
Lysis Reagent (Part # CC-L001-060) solution containing sodium hydroxide, sodium dodecyl sulfate (SDS), and n-butanol.
Extraction Reagent (Part # CC-E001-060) which is a solution of sodium hydroxide.
Neutralization Reagent (Part # CC-N001-060) which is 4-morpholinopropanesulfonate (MOPS) solution with gentamicin.

Instrument States

The instrument has the following states: off state, power-up state, operational state. Transition to power-up state is gated by a manual action, i.e. power switch. Transition to operational state is automatic.

Sample Processing

Before a sample can be processed it must be identified to the system. The instrument will process assays in a serial manner. That is, after the START ASSAY field is actuated, the instrument waits until the user loads a reaction tube into a reaction well indicated by the system.

General

Instrument graphical user interface (GUI)

The instrument is controlled by a GUI.

GUI

The GUI is supported by an embedded PC and is physically incorporated into the BacTx® reader.

Status Display

Users can see a status screen describing assay processing state without logging in. Users log into their account via the LOGIN screen. Account status (login/logout) is shown across the top of all screens. LOGOUT buttons are available on the top level of all screens. LOGIN buttons are available on the top of all screens if no one is logged in.

User Notifications

Alerts

As BacTx® reader runs the user may be notified (visual and/or audio) with a descriptive message, the user cannot silence the notification (without resolving the problem). At some point the user performs an action that fixes the problem and this clears notification. The Alert volume and duration is configurable by the laboratory supervisor in a software setup routine.

User is notified, does not resolve problem
1. Alert occurs (audible & visual).
2. User doesn't resolve the notification, Notification continues, unless duration is specified.

User is Notified, Silences and Clears
1. Alert occurs (audible & visual).
2. Alert data is stored in Instrument/Installation History.
3. Alert continues.
4. User acknowledges the Alert.
5. Audible component stops, popup is removed (silence).
6. Current status still describes the alert is ongoing.
7. Time passes.
8. User corrects problem.
9. The associated alerts caused by this problem are cleared.
10. Alert resolution data is stored in Instrument/Installation History

Configuration and Power-Up

Power-Up: BIT Failed

During power-up, software is used to perform built in tests. These tests could include hardware tests, connectivity monitoring, or software reliability and compatibility tests. Should a BIT test fail, the notification system reports the problem.

Power-Up: Calibration Failures

During power-up one or more optical channels fails calibration. The system notifies the user and locks out those optical channels that have failed. If all channels fail the system cannot be used.

Power-Up: SW Update

Software update is performed from within the power-up routine. The system checks the USB fob for the presence of a software update file, compares the versioning of the update file to the current software version and then proceeds to update the system if warranted.

Power-Up: SW Update, GUI

GUI applications can be either incompatible after an update, or simply not offer all of the new functionality supported. The GUI is updated by the user turning the system off and then on.

Instrument Configuration

The GUI will contain a page (e.g., Miscellaneous) that allows Supervisors to configure the GUI. There are two types of configuration fields: runtime and setup. A runtime configurable field can be implemented without rebooting. To change a setup configurable field the instrument must be rebooted.
1. Runtime configurable fields: Accounts (create, delete, edit), date, time, alert volume, audible alert duration.
2. Setup configurable fields: audible alarm status (on/off)

User Operations (Refer to Login Section)

Assays, Controls and Stat Samples

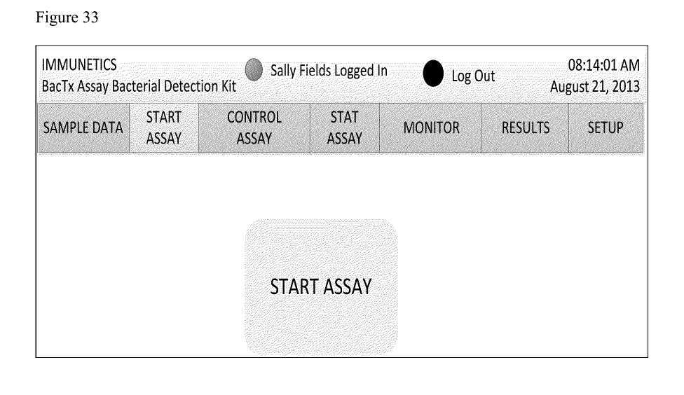

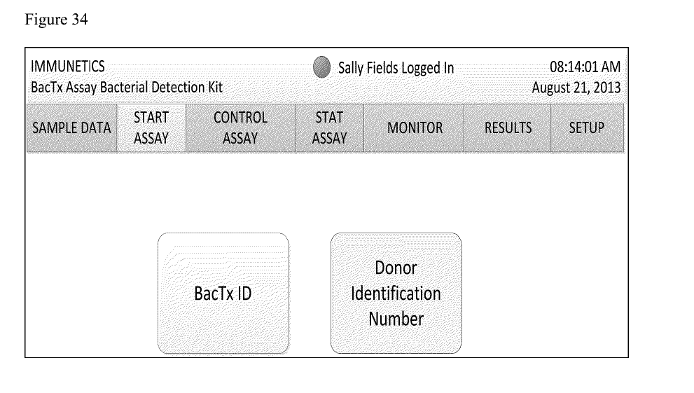

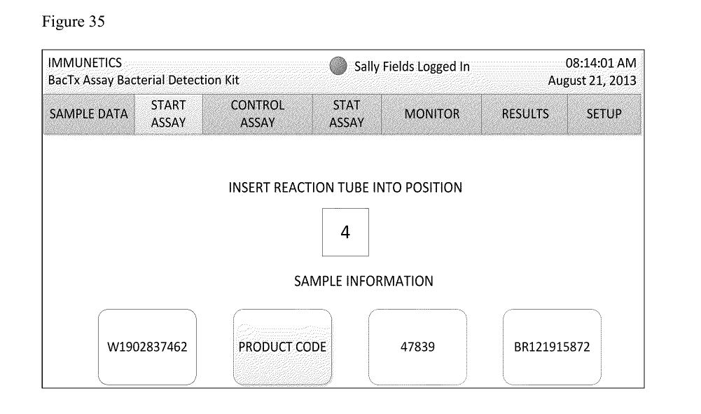

Run Assay: Nominal Use Case with BacTx® ID Label
1. New platelet units are received in the laboratory.
2. User places BacTx® ID labels on the (a) disposable pigtail used to aseptically collect sample from the platelet unit; (b) sterile 2 mL microcentrifuge tubes; (c) sterile 1.5 mL microcentrifuge tubes; and (d) reagent tubes.
3. Users scan the DIN code from a platelet unit (option to scan the PRODUCT INFORMATION CODE field aka PIC at this time as well) and then the affixed BacTx® ID label to associate the BacTx® ID label with the platelet sample.
4. User processes samples as directed by the BacTx® package insert.
5. User activates the START ASSAY field on the ASSAY GUI page. (see FIG. 33)
6. GUI presents the user with the following two fields: the BacTx® ID field and the field. (see FIG. 34)
7. User scans the BacTx® ID Label to populate both the BacTx® ID field and the field (populates the PIC field if it has been scanned in step 3).
8. User removes 300 μl of a processed sample and transfers it into a reagent tube.
9. User mixes the reaction tube for 3 seconds in a vortex.
10. User places the reaction tube in the reaction well indicated on the GUI. (see FIG. 35)
11. User closes the lid on the reaction well that contains the reaction tube.
12. Assay processing is initiated.

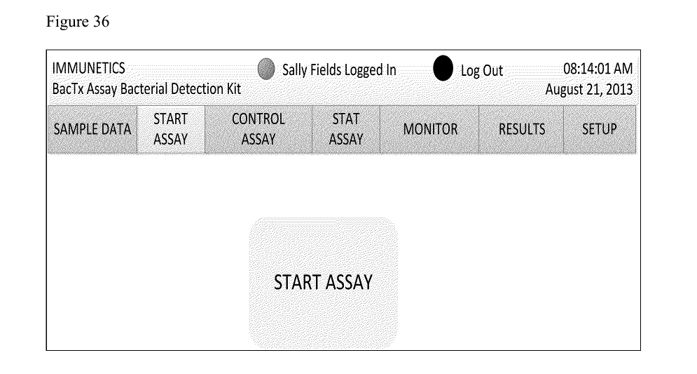

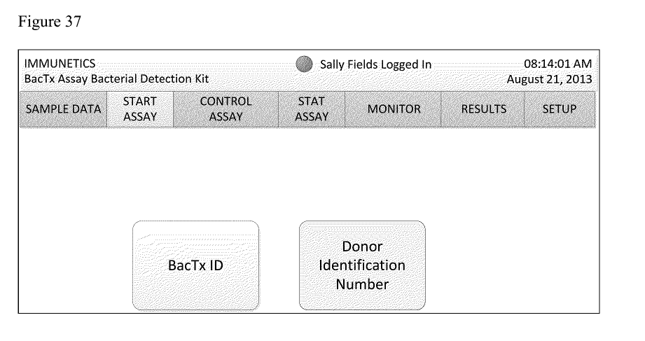

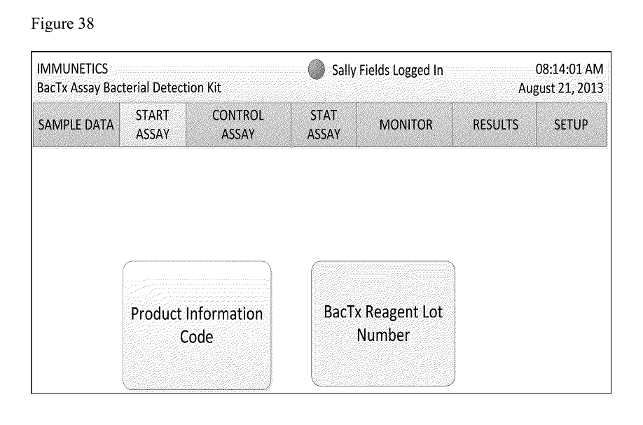

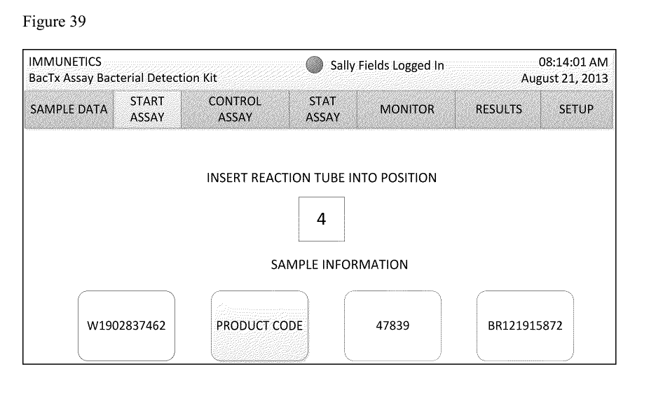

Run Assay: Populate with Scan of Platelet Unit DIN Label
1. New platelet units are received in the laboratory.
2. Users collect disposable sample in pigtail aseptically from the platelet unit.
3. User processes samples as directed by the manufacturer's package insert.
4. User activates the START ASSAY field on the ASSAY GUI page. (see FIG. 36)
5. GUI presents the user with the following two fields: the BACTX® ID LABEL field and the field. (see FIG. 37)
6. User scans the field of the platelet unit.
7. The GUI presents the user with the following two fields: the PRODUCT INFORMATION CODE field and the BACTX® REAGENT LOT NUMBER field. (see FIG. 38)
8. User populates the PRODUCT INFORMATION CODE field (this is optional).
9. User populates the BACTX® REAGENT LOT NUMBER by scanning the reagent tube lot ID label (this is optional).
10. User removes 300 μl of a processed sample and transfers it into a reagent tube. (see FIG. 39)
11. User mixes the reaction tube for 3 seconds in a vortex.
12. User places the reaction tube in the reaction well indicated on the GUI.
13. User closes the lid on the reaction well that contains the reaction tube.
14. Assay processing is initiated when the reaction tube is detected in the indicated reaction well.

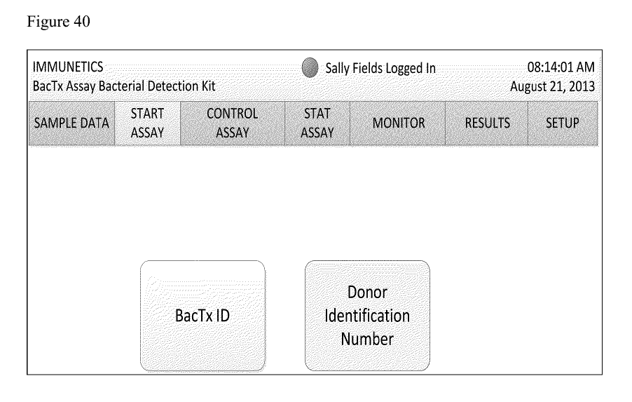

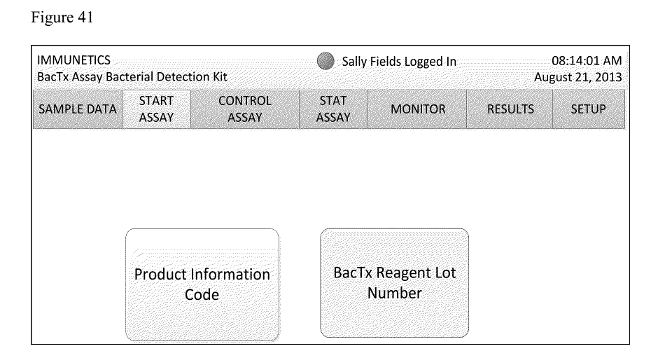

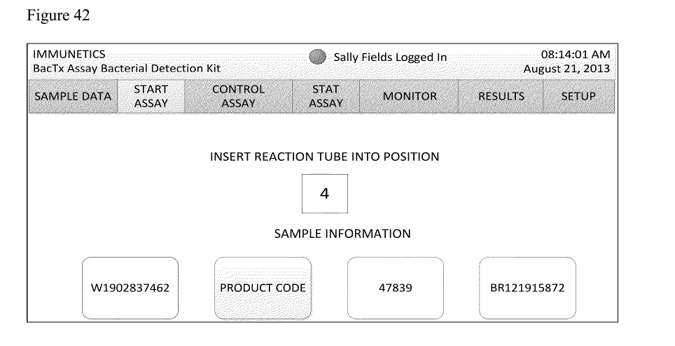

Run Assay: Populate Manually
1. Complete steps 1 to 5 in Use Case 0. (see FIG. 40)
2. User touches the field and populates the DIN using the onscreen keyboard. (see FIG. 41)
3. User populates the PRODUCT INFORMATION CODE field using the onscreen keyboard (this is optional).
4. GUI presents the BACTX® REAGENT LOT NUMBER field to user.
5. User populates the BACTX® REAGENT LOT NUMBER by scanning the reagent tube lot ID label (this is optional) or by using the on-screen keyboard.
6. User removes 300 μl of a processed sample and transfers it into a reagent tube. (see FIG. 42)
7. User mixes the reaction tube for 3 seconds in a vortex.
8. User places the reaction tube in the reaction well indicated on the GUI.
9. User closes the lid on the reaction well that contains the reaction tube.
10. Assay processing is initiated when the reaction tube is detected in the indicated reaction well.

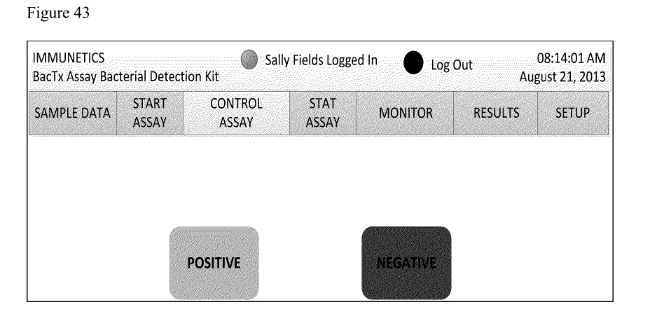

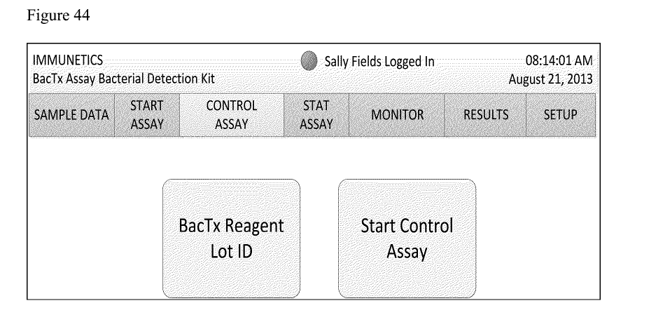

Run Control Assay
1. User activates the CONTROL ASSAY field on the ASSAY GUI page. (see FIG. 43)
2. Users selects the POSITIVE or NEGATIVE field.
3. User populates the BACTX® REAGENT LOT NUMBER by scanning the reagent tube lot ID label (this is optional). (see FIG. 44)
4. User removes 300 μl of a positive or negative control and transfers it into a reagent tube.
5. User mixes the reaction tube for 3 seconds in a vortex.
6. User places the reaction tube in the reaction well indicated on the GUI.
7. User closes the lid on the reaction well that contains the reaction tube.
8. Control processing is initiated when the reaction tube is detected in the indicated
reaction well.

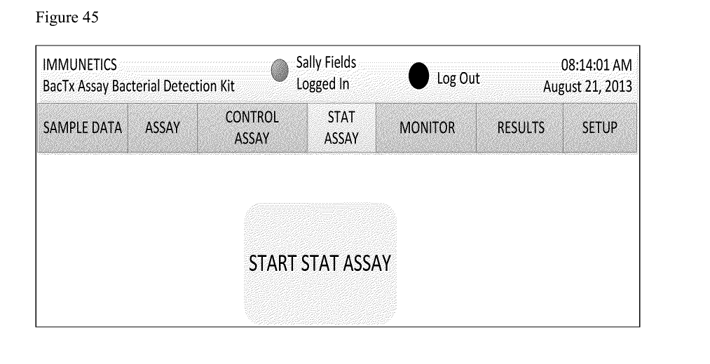

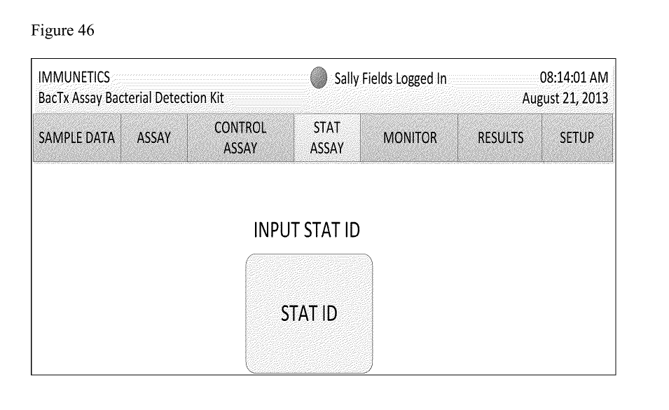

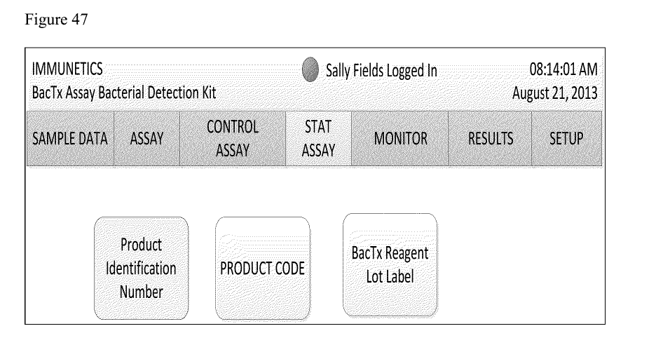

Run Stat Assay
1. User activates the STAT ASSAY field on the ASSAY GUI page. (see FIG. 45)
2. User activates the START STAT ASSAY field.
3. GUI requests user to populate the STAT ID field. (see FIG. 46)
4. The user populates the STAT ID field by one of the following methods:
   User keys in a Stat ID using the onscreen keyboard.
   Scan from platelet unit.
   User scans the BacTx® ID Label, if the label has previously been associated with, a STAT ID field will be populated.
5. User can then populate the PRODUCT IDENTIFICATION CODE and the BACTX® REAGENT LOT NUMBER (by scanning) but both of these fields are optional. (see FIG. 47)
6. User removes 300 μl of Stat sample and transfers it into a reagent tube.
7. User mixes the reaction tube for 3 seconds in a vortex.
8. User places the reaction tube in the reaction well indicated on the GUI.
9. User closes the lid on the reaction well that contains the reaction tube.
10. Stat processing is initiated.

Login/Logout

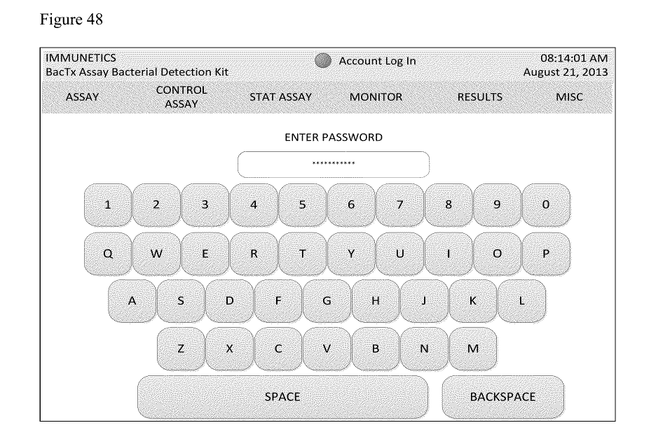

Login
1. User navigates to the ACCOUNT LOGIN page by LOGIN button that is present in top bar of several of the GUI screens. (see FIG. 48)
2. User activates the ENTER PASSWORD field.
3. User populates the ENTER PASSWORD field using the onscreen keyboard.
4. User logs in to their account.

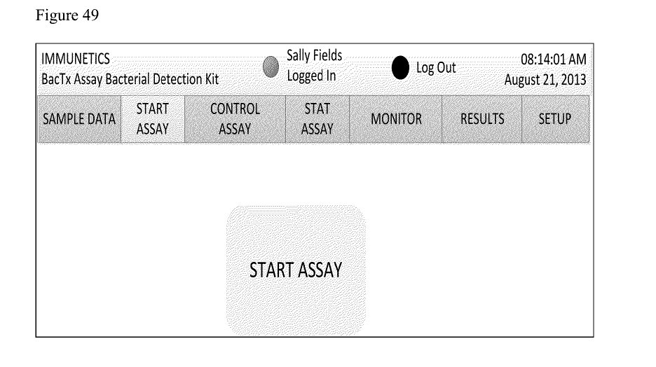

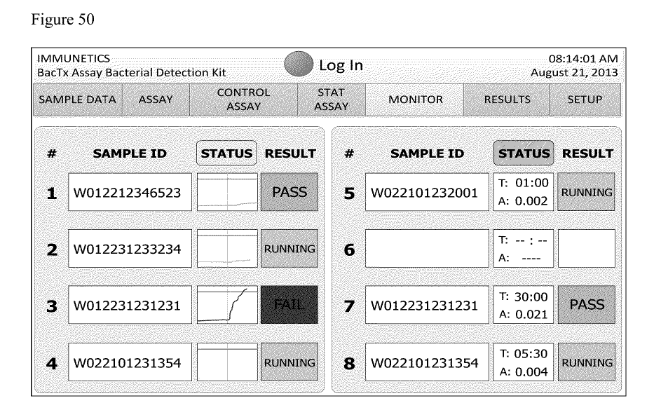

Log Out
1. User activates the LOGOUT button the top bar in the GUI. (see FIG. 49)
2. User is logged out of the GUI.
3. GUI displays the MONITOR page. (see FIG. 50)

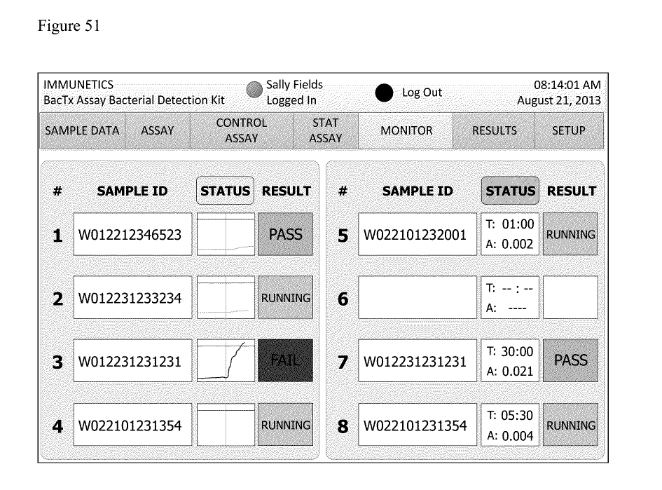

User Monitors Assays
1. The user activates the MONITOR field on the GUI to review assay status.
2. The GUI displays the MONITOR page. (see FIG. 51)
3. User monitors assay status for all current and last assays in well.

Results Page

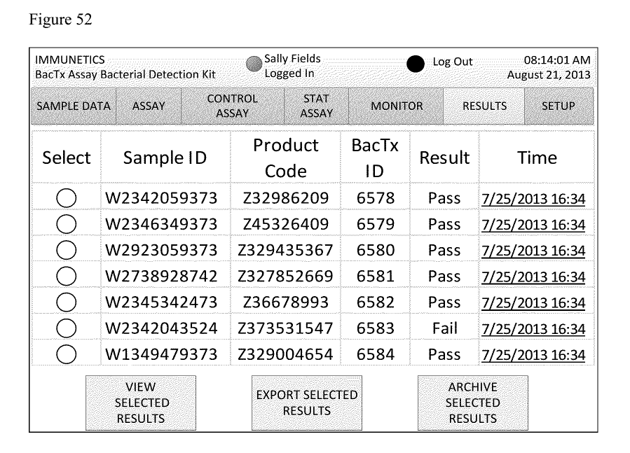

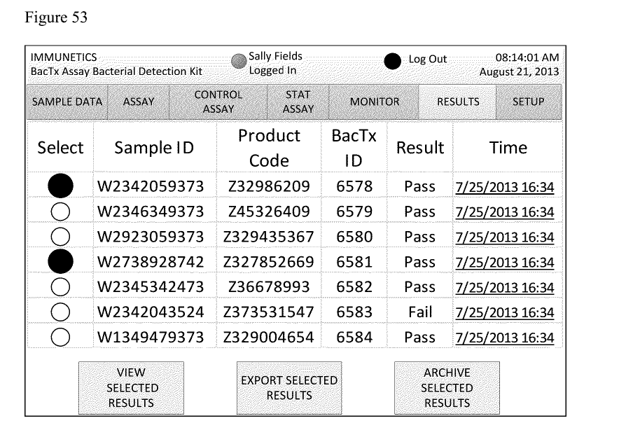

User Exports Results to USB FOB (or Via Other Communications Modules)
1. The user activates the RESULTS field on the GUI.
2. The GUI displays the RESULTS page which is scrollable.
3. The user inserts a formatted USB FOB into the front USB port of the BacTx® reader. (see FIG. 52)
4. The user selects the assay results of interest. (see FIG. 53)
5. The user activates the EXPORT SELECTED RESULTS field.
6. The system writes the selected results to the USB FOB.

User Archives Assay Results
1. The user activates the RESULTS field on the GUI.
2. The GUI displays the RESULTS page.
3. The user selects the assay results of interest.
4. The user activates the ARCHIVE SELECTED RESULTS field.
5. The system writes the selected results to memory.

User View Details of Assay Results
1. The user activates the RESULTS field on the GUI.
2. The GUI displays the RESULTS page.
3. The user selects the assay results of interest.
4. The user activates the VIEW SELECTED RESULTS field.
5. The GUI displays all absorbance readings for the selected assays in addition to the following fields: sample ID, product information code, result, time of assay start, name of account holder who initiated the assay, reagent lot number, control status (run/not run).
6. A print option may be provided.

Misc

Description of Misc from GUI, which may be accessed on the Setup Page by Lab Supervisors or Manager level users.

The MISCELLANEOUS page provides functionality that allows a supervisor to manage user accounts. The MISCELLANEOUS page provides functionality that allows a supervisor to configure certain aspects of GUI behavior. The MISCELLANEOUS page provides functionality that allows a technician to configure the following fields: Date, Time, Audible Alarm Volume and Audible Alarm Duration. Add screen display of MISC fields.

Fields in Miscellaneous Page (Table 1 below)

| | | Required to | | Configurable | |
|---|---|---|---|---|---|
| Screen | | Perform a | Account | R = runtime | Not Seen on |
| Function | Fields | Test | Rights | S = setup | GUI |
| LOG IN | User ID | X | | | |
| | Password | X | | | |
| START | DIN | X | | | |
| ASSAY | PIC | | | | |
| | BacTx ® ID | | | | |
| | BacTx ® Reagent | | | | |
| | Lot | | | | |
| | Insert Rxn Tube | X | | | |
| | Position | | | | |
| CONTROL | Positive | | | | |
| ASSAY | Negative | | | | |
| | BacTx ® Reagent | | | | |
| | Lot | | | | |
| | Insert Rxn Tube | X | | | |
| | Position | | | | |
| | Start Assay | | | | |
| STAT | DIN | X | | | |
| ASSAY | PIC | | | | |
| | BacTx ® ID | | X | | |
| | BacTx ® Reagent | | X | | |
| | Lot | | | | |
| | Insert Rxn Tube | X | | | |
| | Position | | | | |
| | Start Assay | | | | |
| MONITOR | Well Number | | | | |
| | DIN | | | | |
| | PIC | | | | |
| | STATUS | | | | |
| | Time Remaining | | | | |
| | Absorbance | | | | |
| | Current | | | | |
| | Result | | | | |
| | Pass | | | | |
| | Fail | | | | |
| | Running | | | | |
| | Aborted | | | | |
| | Alarm - per channel | | | | |
| RESULTS | Select | X | | | |
| | DIN | | | | |
| | PIC | | | | |
| | Result | | | | |
| | Time | | | | |
| | View Selected | | | | |
| | Results | | | | |
| | Export Selected | | | | |
| | Results | | | | |
| | Archive Selected | | | | |
| | Results | | | | |
| MISC | Self Test | | | | |
| | Auto Log Out Delay | | | S | |
| | Time | | | | |
| | ACCOUNTS | | X | | |
| | Add New | X | | | |
| | Account | | | | |
| | Delete Account | | X | | |
| | Modify Account | | X | | |
| | (ID, PW . . . ) | | | | |
| | Name First | | X | | |
| | Name Last | | X | | |
| | Account Type | | X | | |
| | Account Rights | | X | | |
| | Print Log Report to | | | | |
| | File | | | | |
| | Print User Account | | | | |
| | Report to File | | | | |
| | Export Reports to | | | | |
| | USB | | | | |
| | Date | | | R | |
| | Time | | | R | |
| | Audible Alarm | | | S | |
| | Status | | | | |
| | Alarm Volume | | X | S | |
| | Alarm Duration | | X | S | |
| | Instrument Logs | | | | |

### Example 2

### Sample Pre-Processing and BacTx® Processing Stages

Tables 2.1-2.5 depict a step-wise chart of the various pre-processing and BacTx® processing stages. The pre-processing stage comprises the steps of preparing the sample. The processing stage once the sample has been prepared, placed in a reaction tube, inserted into the optical reader apparatus, and testing is initiated. The various tables show various assay formats such as manual processing (Table 2.1), full-logged processing (Table 2.2), partial logged processing (Table 2.3), STAT processing (Table 2.4), and retesting processing (Table 2.5). Each table details the pre-processing step for preparing the sample per assay formats and the processing steps of the sample in the optical reader apparatus. Also provided are steps for troubleshooting and instructions regarding functional operation, error, and exceptions and comments and notes regarding same that may occur during the stages.

TABLE 2.1

| PROCESSING | STEP | | Manual Processing (Bench | Functional Operation and | |
|---|---|---|---|---|---|
| STAGE | NO. | STEP DESCRIPTION | Protocol) | Errors & Exceptions | COMMENTS & NOTES |
| Platelet Arrival | 0 | Event | NA | No Action | Platelet Unit from HOLD to INVENTORY Status |
| Pre-Processing | 0.1 | Label Tubes | Pre-label tube sets | Apply BacTx labels to processing tubes, | Preparation in advance of the testing procedure. |
| (Preparation for Testing) | | | | grouped by color and 3-digit code. | |
| | 0.2 | Log Into Cascade | No manual processing equivalent | Enter User-specific USERID and | Should allow multiple user logins: Supervisor |
| | | | | PASSWORD. Any samples entered once a | override and ability to log out other users. User |
| | | | | user is logged in will be tied to that user. | inactivity for xx minutes (Supervisor configurable |
| | | | | The record for those samples will then | setting) should act like a password-protected screen |
| | | | | report the user. | saver. |
| | 0.3 | Mode Selection | No manual processing equivalent | Three options given to the user: Control, | STAT mode can be deactivated by the Supervisor. In |
| | | | | Routine, STAT | this case, it will be inaccessable to the standard |
| | | | | | user. |
| | 0.4 | Scan/Enter Platelet ID | Record Unit Number AND Product Code | This field must recognize and accept the | Provision for barcode or manual entry of key data; |
| | | | (i.e bag-specific code) | platelet ID format only, unless Supervisor | ensure the procedure does not depend on a barcode |
| | | | | override. Touching field should bring up | or active/working reader |
| | | | | virtual keyboard, but using barcode | |
| | | | | reader should automatically populate | |
| | | | | field. | |
| BacTx Processing | 1.0 | Draw Platelet Sample | Draw sample Into tube on platelet bag | This field must recognize and accept the | May have Platelet ID or BacTxID (If label with |
| | | | and heat seal. | platelet ID format (9 digits?) and the | Platelet ID is not available) |
| | | | | BacTx ID format. Touching field should | |
| | | | | bring up virtual keyboard, but using | |
| | | | | barcode reader should automatically | |
| | | | | populate field. | |
| | 1.1 | Record Sample Tube ID | Record Sample Tube ID on Log Sheet or | Touching field should bring up virtual | Sample Tube ID may be the ISBT barcode duplicate |
| | | | Notebook, ensure this number | keyboard, but using barcode reader | from or derived from the Platelet bag or (if this does |
| | | | corresponds to the correct Platelet bag | should automatically populate field. | not exist), a BacTx barcode color-coded to the |
| | | | ID number. | | sample. |
| | 1.2 | Query for New/Additional | Record all additional Sample Tube IDs | | |
| | | Samples | with corresponding Platelet BagIDs | | |
| | 1.3 | Aliquot Lysis buffer into 2.0 ml | | | |
| | | tubes and Neutralization Buffer | | | |
| | | into 1.5 ml tubes | | | |
| | 1.4 | Transfer Platelet Test Sample to | For each test sample in turn, transfer | If the “sample set” barcode fields don't | Full barcode utilization (scanning both the source |
| | | LYSIS Tube | the sample from Sample Tube to the | match, an error message needs to be | and receiving tube) ensures the audit trail/chain of |
| | | | BacTxLysis sample processing tube. | generated during full logged processing. | custody/ID of the test material is preserved. for |
| | | | | | STAT, this procedure can be trimmed to allow |
| | | | | | entering just the Platelet ID and the BacTx substrate |
| | | | | | tube ID/barcode. That would be supervisor- |
| | | | | | configurable, as would full vs partial barcode |
| | | | | | tracking. |
| | 1.5 | Centrifugation and decanting of | | | |
| | | lysis buffer | | | |
| | 1.6 | Resuspend Pellet In Extraction | | | |
| | | buffer | | | |
| | 1.7 | Transfer Sample to 1.5 mL tube | For each test sample in turn, transfer | If the “sample set” barcode fields don't | |
| | | containing Neutralization Buffer | the sample from 2.0 mL Lysis Buffer | match, an error message needs to be | |
| | | | tube to the 1.5 mL Neutralization | generated during full logged processing. | |
| | | | Buffer tube | | |
| | 1.8 | Close Neutralization Buffer tube | | | |
| | | and invert 3 times to mix. | | | |
| | 1.9 | Transfer Sample to BacTx | For each test sample in turn, transfer | If the “sample set” barcode fields don't | For retests, the final 1.0 mL volume is sufficient to |
| | | Reaction Tube | the sample from the 1.5 mL | match, an error message needs to be | run the duplicate retesting. 0.3 ml × 2 |
| | | | Neutralization Buffer tube to the | generated during full logged processing | |
| | | | Reaction Tube | AND during partial logged processing. | |
| | 1.10 | Initiate BacTx Assay | Cascade Reader Indicates which | Possible error messages due to placing | Users have 1 minute to initiate an assay before a |
| | | | channel to use for each sample. | tube into incorrect channel, not placing | prompt pops up, and if the assay is not initiated |
| | | | | tube in channel quickly enough, or not | within another minute, that sample is ABORTED. |
| | | | | closing lid on the channel after | Once assay is initiated, users have 1 minute to close |
| | | | | initiation. | the lid before a prompt pops up, and if the lid is not |
| | | | | | closed within another minute, a note is attached to |
| | | | | | the database. |

TABLE 2.2

| PROCESSING | STEP | | Full Logged Processing | Functional Operation and | |
|---|---|---|---|---|---|
| STAGE | NO. | STEP DESCRIPTION | (Cascade Actions/Entries) | Errors & Exceptions | COMMENTS & NOTES |
| Platelet Arrival | 0 | Event | NA | No Action | Platelet Unit from HOLD to INVENTORY Status |
| Pre-Processing | 0.1 | Label Tubes | Pre-label tube sets | Apply BacTx labels to processing tubes, | Preparation In advance of the testing procedure. |
| (Preparation for Testing) | | | | grouped by color and 3-digit code. | |
| | 0.2 | Log Into Cascade | Enter USERID and PASSWORD | Enter User-specific USERID and | Should allow multiple user logins: Supervisor |
| | | | | PASSWORD. Any samples entered once a | override and ability to log out other users. User |
| | | | | user is logged in will be tied to that user. | inactivity for xx minutes (Supervisor configurable |
| | | | | The record for those samples will then | setting) should act like a password-protected screen |
| | | | | report the user. | saver. |
| | 0.3 | Mode Selection | Select “Routine Mode” | Three options given to the user: Control, | STAT mode can be deactivated by the Supervisor. In |
| | | | | Routine, STAT | this case, it will be inaccessable to the standard |
| | | | | | user. |
| | 0.4 | Scan/Enter Platelet ID | Touch field to activate for scan or | This field must recognize and accept the | Provision for barcode or manual entry of key data; |
| | | | manual entry - 2 fields (Unit Number | platelet ID format only, unless Supervisor | ensure the procedure does not depend on a barcode |
| | | | and Product Code). These two fields | override. Touching field should bring up | or active/working reader |
| | | | form the Composite Key (unique | virtual keyboard, but using barcode | |
| | | | identifier) for the Test Article. | reader should automatically populate | |
| | | | | field. | |
| BacTx Processing | 1.0 | Draw Platelet Sample | Bench action; no Cascade action | This field must recognize and accept the | May have Platelet ID or BacTxID (If label with |
| | | | required. | platelet ID format (9 digits?) and the | Platelet ID is not available) |
| | | | | BacTx ID format. Touching field should | |
| | | | | bring up virtual keyboard, but using | |
| | | | | barcode reader should automatically | |
| | | | | populate field. | |
| | 1.1 | Record Sample Tube ID | Touch ID field to activate, and enter | Touching field should bring up virtual | Sample Tube ID may be the ISBT barcode duplicate |
| | | | manually or scan barcode to | keyboard, but using barcode reader | from or derived from the Platelet bag or (if this does |
| | | | populate field. | should automatically populate field. | not exist), a BacTx barcode color-coded to the |
| | | | | | sample. |
| | 1.2 | Query for New/Additional | Follow procedures 1.0 and 1.1 for | | |
| | | Samples | each sample. | | |
| | 1.3 | Aliquot Lysis buffer into 2.0 ml | Bench action; no Cascade action | | |
| | | tubes and Neutralization Buffer | required. | | |
| | | into 1.5 ml tubes | | | |
| | 1.4 | Transfer Platelet Test Sample to | Scan Sample Tube ID barcode, | If the “sample set” barcode fields don't | Full barcode utilization (scanning both the source |
| | | LYSIS Tube | withdraw sample volume for | match, an error message needs to be | and receiving tube) ensures the audit trail/chain of |
| | | | transfer, scan the lysis tube and | generated during full logged processing. | custody/ID of the test material is preserved. for |
| | | | deliver the sample. | | STAT, this procedure can be trimmed to allow |
| | | | | | entering just the Platelet ID and the BacTx substrate |
| | | | | | tube ID/barcode. That would be supervisor- |
| | | | | | configurable, as would full vs partial barcode |
| | | | | | tracking. |
| | 1.5 | Centrifugation and decanting of | Bench action; no Cascade action | | |
| | | lysis buffer | required. | | |
| | 1.6 | Resuspend Pellet In Extraction | Bench action; no Cascade action | | |
| | | buffer | required. | | |
| | 1.7 | Transfer Sample to 1.5 mL tube | Scan Lysis Buffer Tube ID barcode, | If the “sample set” barcode fields don't | |
| | | containing Neutralization Buffer | withdraw sample volume for | match, an error message needs to be | |
| | | | transfer, scan the Neutralization | generated during full logged processing. | |
| | | | Buffer tube and deliver the sample. | | |
| | 1.8 | Close Neutralization Buffer tube | Bench action; no Cascade action | | |
| | | and invert 3 times to mix. | required. | | |
| | 1.9 | Transfer Sample to BacTx | Scan Neutralization Buffer Tube ID | If the “sample set” barcode fields don't | For retests, the final 1.0 mL volume is sufficient to |
| | | Reaction Tube | barcode, withdraw sample volume | match, an error message needs to be | run the duplicate retesting. 0.3 ml × 2 |
| | | | for transfer, scan the BacTx Assay | generated during full logged processing | |
| | | | Reaction tube and deliver the | AND during partial logged processing. | |
| | | | sample. | | |
| | 1.10 | Initiate BacTx Assay | Cascade Reader Indicates which | Possible error messages due to placing | Users have 1 minute to initiate an assay before a |
| | | | channel to use for each sample. | tube into incorrect channel, not placing | prompt pops up, and if the assay is not initiated |
| | | | | tube in channel quickly enough, or not | within another minute, that sample is ABORTED. |
| | | | | closing lid on the channel after | Once assay is initiated, users have 1 minute to close |
| | | | | initiation. | the lid before a prompt pops up, and if the lid is not |
| | | | | | closed within another minute, a note is attached to |
| | | | | | the database. |

TABLE 2.3

| | | | Partial Logged | | |
|---|---|---|---|---|---|
| PROCESSING | | | Processing (Cascade | Functional Operation and | |
| STAGE | STEP NO. | STEP DESCRIPTION | Actions/Entries) | Errors & Exceptions | COMMENTS & NOTES |
| Platelet Arrival | 0 | Event | NA | No Action | Platelet Unit from HOLD to INVENTORY Status |
| Pre-Processing | 0.1 | Label Tubes | Pre-label tube sets | Apply BacTx labels to processing | Preparation In advance of the testing |
| (Preparation for Testing) | | | | tubes, grouped by color and 3-digit | procedure. |
| | | | | code. | |
| | 0.2 | Log into Cascade | Enter USERID and PASSWORD | Enter User-specific USERID and | Should allow multiple user logins; Supervisor |
| | | | | PASSWORD. Any samples entered | override and ability to log out other users. |
| | | | | once a user is logged in will be tied | User inactivity for xx minutes (Supervisor |
| | | | | to that user. The record for those | configurable setting) should act like a |
| | | | | samples will then report the user. | password-protected screen saver. |
| | 0.3 | Mode Selection | Select “Routine Mode” | Three options given to the user: | STAT mode can be deactivated by the |
| | | | | Control, Routine, STAT | Supervisor. In this case, it will be inaccessable |
| | | | | | to the standard user. |
| | 0.4 | Scan/Enter Platelet ID | Touch field to activate for scan | This field must recognize and accept | Provision for barcode or manual entry of key |
| | | | or manual entry - 2 fields (Unit | the platelet ID format only, unless | data; ensure the procedure does not depend |
| | | | Number and Product Code). | Supervisor override. Touching field | on a barcode or active/working reader |
| | | | These two fields form the | should bring up virtual keyboard, but | |
| | | | Composite Key (unique | using barcode reader should | |
| | | | Identifier) for the Test Article. | automatically populate field. | |
| BacTx Processing | 1.0 | Draw Platelet Sample | Bench action; no Cascade action | This field must recognize and accept | May have Platelet ID or BacTx ID (if label with |
| | | | required. | the platelet ID format (9 digits?) and | Platelet ID is not available) |
| | | | | the BacTx ID format. Touching field | |
| | | | | should bring up virtual keyboard, but | |
| | | | | using barcode reader should | |
| | | | | automatically populate field. | |
| | 1.1 | Record Sample Tube ID | Touch ID field to activate, and | Touching field should bring up | Sample Tube ID may be the ISBT barcode |
| | | | enter manually or scan barcode | virtual keyboard, but using barcode | duplicate from or derived from the Platelet |
| | | | to populate field. | reader should automatically | bag, or (if this does not exist), a BacTx barcode |
| | | | | populate field. | color-coded to the sample. |
| | 1.2 | Query for New/Additional | Follow procedures 1.0 and 1.1 | | |
| | | Samples | for each sample. | | |
| | 1.3 | Aliquot Lysis buffer into 2.0 ml | Bench action; no Cascade action | | |
| | | tubes and Neutralization | required. | | |
| | | Buffer into 1.5 ml tubes | | | |
| | 1.4 | Transfer Platelet Test Sample | Transfer sample, no barcode | If the “sample set” barcode fields | Full barcode utilization (scanning both the |
| | | to LYSIS Tube | tracking | don't match, an error message needs | source and receiving tube) ensures the audit |
| | | | | to be generated during full logged | trail/chain of custody/ID of the test material is |
| | | | | processing. | preserved. For STAT, this procedure can be |
| | | | | | trimmed to allow entering just the Platelet ID |
| | | | | | and the BacTx substrate tube ID/barcode. That |
| | | | | | would be supervisor-configurable, as would |
| | | | | | full vs partial barcode tracking. |
| | 1.5 | Centrifugation and decanting | Bench action; no Cascade action | | |
| | | of lysis buffer | required. | | |
| | 1.6 | Resuspend Pellet in | Bench action; no Cascade action | | |
| | | Extraction buffer | required. | | |
| | 1.7 | Transfer Sample to 1.5 mL | Transfer sample, no barcode | If the “sample set” barcode fields | |
| | | tube containing | tracking | don't match, an error message needs | |
| | | Neutralization Buffer | | to be generated during full logged | |
| | | | | processing. | |
| | 1.8 | Close Neutralization Buffer | Bench action; no Cascade action | | |
| | | tube and invert 3 times to | required. | | |
| | | mix. | | | |
| | 1.9 | Transfer Sample to BacTx | Withdraw sample volume for | If the “sample set” barcode fields | For retests, the final 1.0 mL volume is |
| | | Reaction Tube | transfer, scan the BacTx Assay | don't match, an error message needs | sufficient to run the duplicate retesting. 0.3 mL × 2 |
| | | | Reaction tube and deliver the | to be generated during full logged | |
| | | | sample. | processing AND during partial logged | |
| | | | | processing. | |
| | 1.10 | Initiate BacTx Assay | Cascade Reader indicates which | Possible error messages due to | Users have 1 minute to initiate an assay before |
| | | | channel to use for each sample. | placing tube into incorrect channel, | a prompt pops up, and if the assay is not |
| | | | | not placing tube in channel quickly | initiated within another minute, that sample |
| | | | | enough, or not closing lid on the | is ABORTED. Once assay is initiated, users |
| | | | | channel after Initiation. | have 1 minute to close the lid before a prompt |
| | | | | | pops up, and if the lid is not closed within |
| | | | | | another minute, a note is attached to the |
| | | | | | database. |

TABLE 2.4

| PROCESSING | | | STAT Processing | Functional Operation and | |
|---|---|---|---|---|---|
| STAGE | STEP NO. | STEP DESCRIPTION | (Cascade Actions/Entries) | Errors & Exceptions | COMMENTS & NOTES |
| Platelet Arrival | 0 | Event | NA | No Action | Platelet Unit from HOLD to INVENTORY Status |
| Pre-Processing | 0.1 | Label Tubes | Pre-label tube sets | Apply BacTx labels to processing | Preparation In advance of the testing |
| (Preparation for Testing) | | | | tubes, grouped by color and 3-digit | procedure. |
| | | | | code. | |
| | 0.2 | Log into Cascade | Enter USERID and PASSWORD | Enter User-specific USERID and | Should allow multiple user logins; Supervisor |
| | | | | PASSWORD. Any samples entered | override and ability to log out other users. |
| | | | | once a user is logged in will be tied | User inactivity for xx minutes (Supervisor |
| | | | | to that user. The record for those | configurable setting) should act like a |
| | | | | samples will then report the user. | password-protected screen saver. |
| | 0.3 | Mode Selection | Select “STAT Mode” | Three options given to the user: | STAT mode can be deactivated by the |
| | | | | Control, Routine, STAT | Supervisor. In this case, it will be inaccessable |
| | | | | | to the standard user. |
| | 0.4 | Scan/Enter Platelet ID | Touch field to activate for scan | This field must recognize and accept | Provision for barcode or manual entry of key |
| | | | or manual entry-2 fields(Unit | the platelet ID format only, unless | data; ensure the procedure does not depend |
| | | | Number and Product Code). | Supervisor override. Touching field | on a barcode or active/working reader |
| | | | These two fields form the | should bring up virtual keyboard, but | |
| | | | Composite Key (unique | using barcode reader should | |
| | | | Identifier) for the Test Article. | automatically populate field. | |
| BacTx Processing | 1.0 | Draw Platelet Sample | Bench action; no Cascade action | This field must recognize and accept | May have Platelet ID or BacTx ID (if label with |
| | | | required. | the platelet ID format (9 digits?) and | Platelet ID is not available) |
| | | | | the BacTx ID format. Touching field | |
| | | | | should bring up virtual keyboard, but | |
| | | | | using barcode reader should | |
| | | | | automatically populate field. | |
| | 1.1 | Record Sample Tube ID | Touch ID field to activate, and | Touching field should bring up | Sample Tube ID may be the ISBT barcode |
| | | | enter manually or scan barcode | virtual keyboard, but using barcode | duplicate from or derived from the Platelet |
| | | | to populate field. | reader should automatically | bag, or (if this does not exist), a BacTx barcode |
| | | | | populate field. | color-coded to the sample. |
| | 1.2 | Query for New/Additional | Follow procedures 1.0 and 1.1 | | |
| | | Samples | for each sample. | | |
| | 1.3 | Aliquot Lysis buffer into 2.0 ml | Bench action; no Cascade action | | |
| | | tubes and Neutralization | required. | | |
| | | Buffer into 1.5 ml tubes | | | |
| | 1.4 | Transfer Platelet Test Sample | Transfer sample, no barcode | If the “sample set” barcode fields | Full barcode utilization (scanning both the |
| | | to LYSIS Tube | tracking | don't match, an error message needs | source and receiving tube) ensures the audit |
| | | | | to be generated during full logged | trail/chain of custody/ID of the test material is |
| | | | | processing. | preserved. For STAT, this procedure can be |
| | | | | | trimmed to allow entering just the Platelet ID |
| | | | | | and the BacTx substrate tube ID/barcode. That |
| | | | | | would be supervisor-configurable, as would |
| | | | | | full vs partial barcode tracking. |
| | 1.5 | Centrifugation and decanting | Bench action; no Cascade action | | |
| | | of lysis buffer | required. | | |
| | 1.6 | Resuspend Pellet in | Bench action; no Cascade action | | |
| | | Extraction buffer | required. | | |
| | 1.7 | Transfer Sample to 1.5 mL | Transfer sample, no barcode | If the “sample set” barcode fields | |
| | | tube containing | tracking | don't match, an error message needs | |
| | | Neutralization Buffer | | to be generated during full logged | |
| | | | | processing. | |
| | 1.8 | Close Neutralization Buffer | Bench action; no Cascade action | | |
| | | tube and invert 3 times to | required. | | |
| | | mix. | | | |
| | 1.9 | Transfer Sample to BacTx | Transfer sample, no barcode | If the “sample set” barcode fields | For retests, the final 1.0 mL volume is |
| | | Reaction Tube | tracking | don't match, an error message needs | sufficient to run the duplicate retesting. 0.3 mL × 2 |
| | | | | to be generated during full logged | |
| | | | | processing AND during partial logged | |
| | | | | processing. | |
| | 1.10 | Initiate BacTx Assay | Cascade Reader indicates which | Possible error messages due to | Users have 1 minute to initiate an assay before |
| | | | channel to use for each sample. | placing tube into incorrect channel, | a prompt pops up, and if the assay is not |
| | | | | not placing tube in channel quickly | initiated within another minute, that sample |
| | | | | enough, or not closing lid on the | is ABORTED. Once assay is initiated, users |
| | | | | channel after Initiation. | have 1 minute to close the lid before a prompt |
| | | | | | pops up, and if the lid is not closed within |
| | | | | | another minute, a note is attached to the |
| | | | | | database. |

TABLE 2.5

| PROCESSING | | | | Functional Operation and | |
|---|---|---|---|---|---|
| STAGE | STEP NO. | STEP DESCRIPTION | RETEST Scenario | Errors & Exceptions | COMMENTS & NOTES |
| Platelet Arrival | 0 | Event | Failed First Test | No Action | Platelet Unit from HOLD to INVENTORY Status |
| Pre-Processing | 0.1 | Label Tubes | Pre-label duplicate tube sets | Apply BacTx labels to processing | Preparation in advance of the testing |
| (Preparation for Testing) | | | | tubes, grouped by color and 3-digit | procedure. |
| | | | | code. | |
| | 0.2 | Log into Cascade | Enter USERID and PASSWORD | Enter User-specific USERID and | Should allow multiple user logins; Supervisor |
| | | | | PASSWORD. Any samples entered | override and ability to log out other users. |
| | | | | once a user is logged in will be tied | User inactivity for xx minutes (Supervisor |
| | | | | to that user. The record for those | configurable setting) should act like a |
| | | | | samples will then report the user. | password-protected screen saver. |
| | 0.3 | Mode Selection | Processed as per first run, | Three options given to the user: | STAT mode can be deactivated by the |
| | | | selecting the same mode. | Control, Routine, STAT | Supervisor. In this case, it will be inaccessable |
| | | | | | to the standard user. |
| | 0.4 | Scan/Enter Platelet ID | Touch field to activate for scan | This field must recognize and accept | Provision for barcode or manual entry of key |
| | | | or manual entry. The system | the platelet ID format only, unless | data; ensure the procedure does not depend |
| | | | should flag this as already | Supervisor override. Touching field | on a barcode or active/working reader |
| | | | tested, and ask to confirm the | should bring up virtual keyboard, but | |
| | | | Product Code. | using barcode reader should | |
| | | | | automatically populate field. | |
| BacTx Processing | 1.0 | Draw Platelet Sample | Bench action; no Cascade action | This field must recognize and accept | May have Platelet ID or BacTx ID (if label with |
| | | | required. | the platelet ID format (9 digits?) and | Platelet ID is not available) |
| | | | | the BacTx ID format. Touching field | |
| | | | | should bring up virtual keyboard, but | |
| | | | | using barcode reader should | |
| | | | | automatically populate field. | |
| | 1.1 | Record Sample Tube ID | Touch ID field to activate, and | Touching field should bring up | Sample Tube ID may be the ISBT barcode |
| | | | enter manually or scan barcode | virtual keyboard, but using barcode | duplicate from or derived from the Platelet |
| | | | to populate field. | reader should automatically | bag, or (if this does not exist), a BacTx barcode |
| | | | | populate field. | color-coded to the sample. |
| | 1.2 | Query for New/Additional | Follow procedures 1.0 and 1.1 | | |
| | | Samples | for each sample. | | |
| | 1.3 | Aliquot Lysis buffer into 2.0 ml | Bench action; no Cascade action | | |
| | | tubes and Neutralization | required. | | |
| | | Buffer into 1.5 ml tubes | | | |
| | 1.4 | Transfer Platelet Test Sample | Processed as per first run, | If the “sample set” barcode fields | Full barcode utilization (scanning both the |
| | | to LYSIS Tube | following the same | don't match, an error message needs | source and receiving tube) ensures the audit |
| | | | transfer/barcode standard. | to be generated during full logged | trail/chain of custody/ID of the test material is |
| | | | | processing. | preserved. For STAT, this procedure can be |
| | | | | | trimmed to allow entering just the Platelet ID |
| | | | | | and the BacTx substrate tube ID/barcode. That |
| | | | | | would be supervisor-configurable, as would |
| | | | | | full vs partial barcode tracking. |
| | 1.5 | Centrifugation and decanting | Bench action; no Cascade action | | |
| | | of lysis buffer | required. | | |
| | 1.6 | Resuspend Pellet in | Bench action; no Cascade action | | |
| | | Extraction buffer | required. | | |
| | 1.7 | Transfer Sample to 1.5 mL | Processed as per first run, | If the “sample set” barcode fields | |
| | | tube containing | following the same | don't match, an error message needs | |
| | | Neutralization Buffer | transfer/barcode standard. | to be generated during full logged | |
| | | | | processing. | |
| | 1.8 | Close Neutralization Buffer | Bench action; no Cascade action | | |
| | | tube and invert 3 times to | required. | | |
| | | mix. | | | |
| | 1.9 | Transfer Sample to BacTx | Processed as per first run, | If the “sample set” barcode fields | For retests, the final 1.0 mL volume is |
| | | Reaction Tube | following the same | don't match, an error message needs | sufficient to run the duplicate retesting, 0.3 mL × 2 |
| | | | transfer/barcode standard. | to be generated during full logged | |
| | | | | processing AND during partial logged | |
| | | | | processing. | |
| | 1.10 | Initiate BacTx Assay | Cascade Reader indicates which | Possible error messages due to | Users have 1 minute to initiate an assay before |
| | | | channel to use for each sample. | placing tube into incorrect channel, | a prompt pops up, and if the assay is not |
| | | | | not placing tube in channel quickly | initiated within another minute, that sample |
| | | | | enough, or not closing lid on the | is ABORTED. Once assay is initiated, users |
| | | | | channel after Initiation. | have 1 minute to close the lid before a prompt |
| | | | | | pops up, and if the lid is not closed within |
| | | | | | another minute, a note is attached to the |
| | | | | | database. |

### Example 3

### Clinical Testing Results of Assay Methods

The clinical data provided herein reflects use of the BacTx® Assay and kit detect bacteria in both Apheresis Platelets Leukocytes Reduced (LRAP), and pools of up to six (6) units of leukocyte reduced whole blood-derived platelets (LR-WBDP) that are pooled within four (4) hours of transfusion. Use of the assay detection system of the present invention is expected to produce substantially the same sensitivity, reproducibility, accuracy, and functionality as the data presented below.

### Interpretation of Results:

Result Interpretation
PASS: No bacteria detected above assay threshold
FAIL: Bacteria detected above assay threshold.
   The result should be confirmed by retesting in duplicate.

1. The assay detection system will interpret the result for each reaction tube as Pass or Fail automatically. For samples which have not generated a “FAIL” result during the 30 minute test period, the result will be interpreted as “PASS”. A FAIL result will be displayed as soon as it is detected, accompanied by an audible alarm. A result log will be created and saved for each run.

2. A “PASS” result means that no bacteria were detected in the sample above the assay threshold. A “PASS” result is valid for up to 24 hours post-sampling for LRAP. For pools of LR-WBDP the BacTx® Assay is performed within 4 hours prior to transfusion.

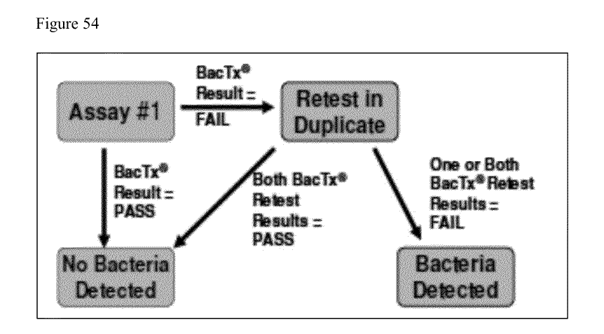

3. A “FAIL” result should be confirmed by re-testing in duplicate. If either of the retests also “FAIL,” this means that bacteria were detected at a concentration above the assay threshold. A flowchart of the BacTx® Assay Testing algorithm is shown in FIG. 54.

4. An immediate ABORT displayed by the Optical reader apparatus within 30 seconds of initiating an assay (i.e. within 30 seconds of pressing the “Start All Samples” button) may indicate a highly contaminated platelet unit that is too turbid to be read by the optical reader apparatus. In the case of an immediate ABORT, dilution and retesting of the sample is recommended as follows: Using a clean, sterile pipette, place 0.5 mL of Extraction Buffer into a 1.5 mL microfuge tube. Add 0.5 mL of Neutralization Buffer to the tube and pipette up and down 5 times to mix. Add 0.1 mL of the remaining platelet extract from the platelet sample that ABORTED. Close the lid of the tube and vortex for 3 seconds. Add 0.3 mL of the diluted sample to a new BacTx® Reaction Tube and perform the BacTx® Assay following the standard procedure.

5. Deviations from the procedure may lead to aberrant results. Results from assays with protocol deviations should be invalidated and the assay repeated.

6. For potentially interfering substances, see the “Performance Characteristics” section below.

### Analytical Sensitivity Study

### LRAP Study Description:

The limit of detection of the BacTx® Assay was determined for 10 species of bacteria (four Gram-positive aerobes, four Gram negative aerobes, and 2 anaerobes). Spiking studies were performed at two external sites. Three lots of BacTx® Kits were used during the analytical sensitivity testing. Bacterial concentrations in LRAPs were estimated by optical density to be between 1×10³ CFU/mL and 1×10⁵ CFU/mL. The actual titer was confirmed by quantitative plate culture. The lowest bacterial concentration at which 10 out of 10 replicates of the BacTx® Assay were positive for bacterial contamination (i.e. 10 out of 10 “FAIL” results) was recorded, and the higher value between the two clinical sites was taken to be the limit of detection (see Table 3.1).

### LR-WBDP Study Description:

The limit of detection of the BacTx® Assay was determined for 10 species of bacteria (four Gram-positive aerobes, four Gram negative aerobes, and 2 anaerobes). Spiking studies were performed at two external sites. Four lots of BacTx® Kits were used during the analytical sensitivity testing. Bacterial concentrations in the pooled platelets were estimated by optical density to be between 1×10³ CFU/mL and 1×10⁵ CFU/mL. The actual titer was confirmed by quantitative plate culture. The lowest bacterial concentration at which 10 out of 10 replicates of the BacTx® Assay were positive for bacterial contamination (i.e. 10 out of 10 “FAIL” results) was recorded, and the higher value between the two clinical sites was taken to be the limit of detection (see

TABLE 3.1
Analytical Sensitivity of the BacTx ®Assay

| | Gram- | | | | |
|---|---|---|---|---|---|
| | Positive | | | | |
| | (GP) or | | | Limit of | Limit of |
| | Gram- | Aerobe | | Detection with | Detection with |
| | Negative | or | ATCC | LR-WBDP | LRAP |
| Species | (GN) | Anaerobe | Number | (CFU/mL) | (CFU/mL) |
| Escherichia coli | GN | Aerobe | 25922 | 8.7 × 10³ | 7.6 × 10⁴ |
| Pseudomonas aeruginosa | GN | Aerobe | 27853 | 5.0 × 10⁴ | 2.7 × 10⁴ |
| Klebsiella oxytoca | GN | Aerobe | 43863 | 9.9 × 10³ | 1.6 × 10⁴ |
| Serratia marcescens | GN | Aerobe | 43862 | 5.8 × 10⁴ | 5.3 × 10³ |
| Bacillus cereus | GP | Aerobe | 11778 | 1.7 × 10³ | 1.9 × 10³ |
| Staphylococcus aureus | GP | Aerobe | 27217 | 4.0 × 10³ | 2.2 × 10³ |
| Staphylococcus epidermidis | GP | Aerobe | 49134 | 2.4 × 10³ | 1.3 × 10³ |
| Streptococcus agalactiae | GP | Aerobe | 12386 | 2.7 × 10⁴ | 4.5 × 10³ |
| Clostridium perfringens | GP | Anaerobe | 3629 | 4.5 × 10³ | 4.8 × 10³ |
| Propionibacterium acnes | GP | Anaerobe | 11827 | 7.2 × 10³ | 8.5 × 10³ |

### Time to Detection (Bacterial Growth) Study

### LRAP Study Description:

To determine the time to detection of bacteria growing in LRAP units, low titers (1.3-5.3 CFU/mL) of bacteria were spiked into LRAP units and allowed to proliferate for 7 days. The same bacterial strains used in the analytical sensitivity study above (See Table 3.1) were used for the Time to Detection Study. In order to ensure growth of bacteria in LRAPs, for each strain tested, four LRAP units were spiked, the LRAP that best supported growth was used for testing, and the other units were discarded. An LRAP spiked with sterile PBS were used as a negative control and also incubated for the 7 days. At approximately 48 hours after inoculation, a small volume of platelets was withdrawn from the contaminated and uncontaminated LRAPs. Ten samples from the contaminated LRAP and three samples from the uncontaminated LRAP were blinded and tested with the BacTx® Assay. If less than 10 of the contaminated samples were detected at the 48 hour time point, this testing was repeated at approximately 72 hours after inoculation. All LRAPs were also tested at approximately 7 days after inoculation. When BacTx® testing was performed, quantitative plate culture (QPC) was carried out to determine the bacterial titer in the contaminated LRAP unit at that time point. Culture plates made at 24 hours and 7 days after inoculation from the spiked units were submitted for bacterial identification to confirm the strain that proliferated in the LRAP was the same as the strain that was inoculated. BacT/ALERT® testing was performed at day 0 to confirm sterility of the LRAP unit. Testing was conducted at two sites with multiple lots of BacTx® Assay Kits.

### LRAP Study Results:

The results of the BacTx® testing and quantitative plate culture are shown in Table 3.2. Of the 8 aerobes tested, six species were detected at 48 hours at both sites. S. agalactiae, was detected at 48 hours at one site and at 72 hours at the second site. P. aeruginosa was detected at 72 hours at both sites. As expected, Clostridium perfringens did not grow during the Time to Detection study. Propionibacterium acnes was not detected by the BacTx® Assay or by Quantitative Plate Culture during the Time to Detection study. Based on these results, the optimal time to detection for all of the bacterial strains that proliferate in platelet units is 72 hours.

TABLE 3.2
Summary of BacTx ® testing and quantitative plate culture results for LRAP time-to-detection study

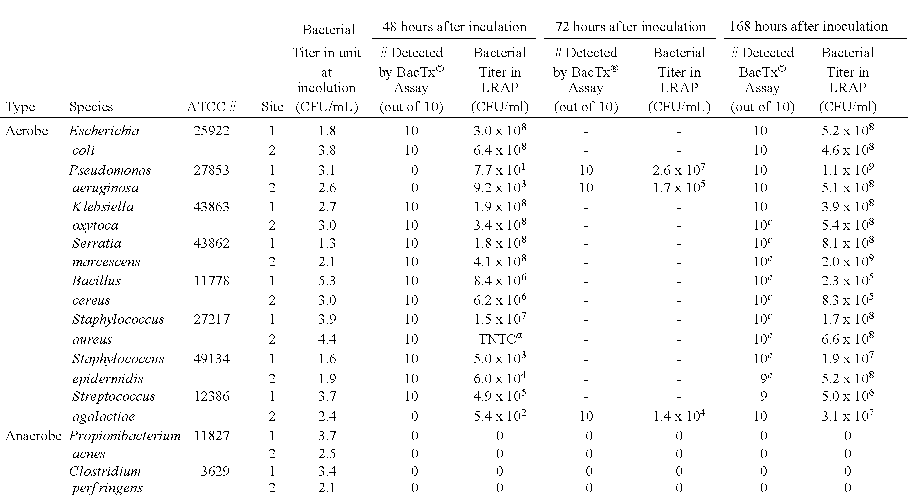

aTNTC = Too numerous to count after dilution placing.
bClostridium perfringens did not grow during the Time to Detection study. Propionibacterium acnes was not detected by the BacTx® Assay or by Quantitative
Place Culture during the Time to Detection study.
cOne or more processed samples gave an ABORT result, due to high sample turbidity. In these instances, the results represented are for an 11-fold dilution of the
processed sample, as described in the ation on INTERPRETATION OF RESULTS.
The first time point at which 10 out of 10 samples were detected by the BacTx® Assay is shaded in grey.

### LR-WBDP Study Description:

To determine the time to detection of bacteria growing in LR-WBDP units, low titers (0.6-5.0 CFU/mL) of bacteria were spiked into individual LR-WBDP units and incubated on a platelet shaker for 7 days. The same bacterial strains used in the analytical sensitivity study above (See Table 3.1) were used for the Time to Detection Study. Platelet units spiked with sterile PBS were used as a negative control and also incubated for the 7 days. At approximately 48 hours after inoculation, a small volume of platelets was withdrawn from the contaminated and uncontaminated units. These volumes were each combined with volumes from 5 other sterile LR-WBDP units in order to create contaminated and uncontaminated platelet pools, respectively. Ten samples from the contaminated pool and three samples from the uncontaminated pool were blinded and tested with the BacTx® Assay. If less than 10 of the contaminated samples were detected at the 48 hour time point, this testing was repeated at approximately 72 hours after inoculation. All units were also tested at approximately 7 days after inoculation. When BacTx® testing was performed, quantitative plate culture was carried out to determine the bacterial titer in the contaminated pool at that time point. Culture plates made at 24 hours and 7 days after inoculation from the spiked units were submitted for bacterial identification to confirm the strain that proliferated in the unit was the same as the strain that was inoculated. Testing was conducted at two sites with multiple lots of BacTx® Assay Kits.

### LR-WBDP Study Results:

The results of the BacTx® testing and quantitative plate culture are shown in Table 3.3. All titer values at 48, 72, and 168 hours reflect the concentration after pooling. Of the eight aerobic strains tested, seven of the strains were detected at 48 hours, with 159 of the 160 contaminated platelet detected by the BacTx® Assay at this time point. All 10 samples of the contaminated pool containing S. epidermidis were detected at the 72 hour time point. Neither of the two anaerobes tested (C. perfringens and P. acnes) exhibited any detectable growth in the aerobic environment of the platelet unit over the 7 day study, and were not detected by the BacTx® Assay. Based on these results, the time to detect all eight aerobes is 72 hours after collection.

TABLE 3.3
Summary of BacTx ® Testing and Quantitative plate culture results for LR-WBDP time-to-detection study
Table 3. Summary of BacTx ® Testing and Quantitative Plate Culture Results for LR-WBDP Time-to-Detection Study

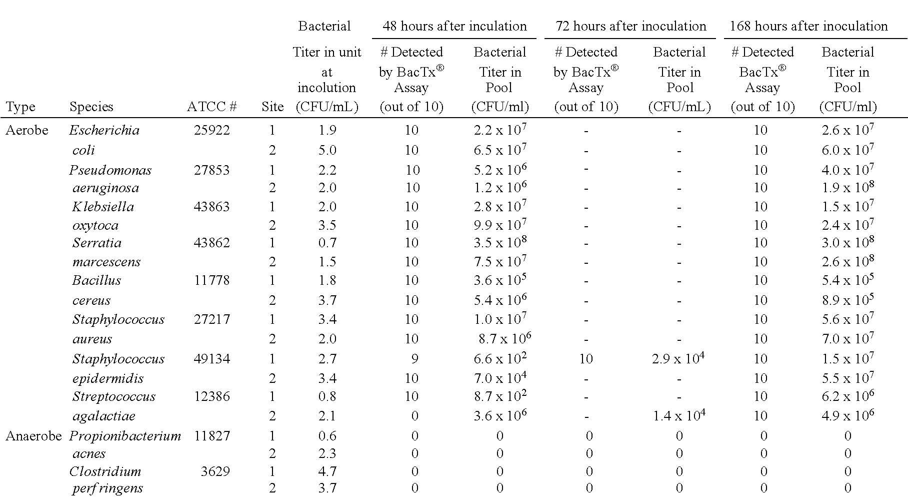

^{a}Second of two TTD studies performed at Site 1 with S. agalactiae. In the first attempt, S. agalactiae did not readily proriferate in the platelet unit, with measurement titers
of 60 and 660 CFU/mL at 48 and 72 hours after inoculation, respectively. A Time-to-Detection within the 5 day shelf-life of the platelet unit could not be deteremined and this
study was repeated.
The first time point at which 10 out of 10 samples were detected by the BacTx® Assays is shaded in grey.

### Specificity Study

### LRAP Study Description:

Specificity of the BacTx® Assay was tested at two external sites using six lots of BacTx® Assay Kits and 505 unique LRAP units. Sterility of the platelet units were confirmed by culture on blood agar plates. This study also served as a test of reproducibility of negative assays, as described in the “reproducibility study” section.

### LRAP Study Results:

505 LRAP units were tested, of which 501 were negative for the presence of bacteria in the BacTx® Assay (BacTx® Assay result=PASS.) Of the 4 LRAP units (0.79%) that were positive in initial testing, 3 were negative in duplicate retests. Thus 1 LRAP unit out of 505 was Repeat Reactive. This corresponds to a specificity, defined as (1—the frequency of Repeat Reactive samples) of 99.8%, with a lower one-sided 95% confidence limit of 99.1%.

### LR-WBDP Study Description:

Specificity of the BacTx® Assay was tested at two external sites using three lots of BacTx® Assay Kits and 432 unique, 6-unit platelet pools. Sterility of the platelet pools was confirmed sterile by plate culture. This study also served as a test of reproducibility of negative assays, as described in the “reproducibility study” section.

### LR-WBDP Study Results:

Out of the 432 BacTx® Assays, 431 were negative for the presence of bacteria in the BacTx® Assay (i.e. a “PASS” result), corresponding to a specificity of 99.8% (with a lower one-sided 95% confidence limit of 99.0%).

### Reproducibility Study

### LRAP Study Description:

Reproducibility of the BacTx® Assay Kit was determined inter-assay, inter-lot, and inter-site for both negative and positive (spiked) LRAPs. The analytical sensitivity study above served as an inter-assay reproducibility study for spiked LRAPs, as 10/10 positive BacTx® Assay results were required to be positive for the presence of bacteria at a given titer before the assay was validated as positive. For the inter-assay reproducibility of negative BacTx® assays, 21 unique, sterile LRAP units were tested with 3 kit lots at a single site with 10 replicates of the BacTx® Assay for a total of 210 assays. For the inter-lot and intersite reproducibility, a test panel was used for testing at three sites using three lots of BacTx® Assay Kits on three different days. The test panel consisted of 10 bacterial members and one negative member, and the composition of the positive panel members is listed in Table 4. The sterility of LRAPs used in the study was confirmed by either BacT/ALERT® culture or plate culture.

### LRAP Study Results:

For the inter-assay reproducibility of negative assays, 210 out of 210 assays gave the expected negative result (100% concordance with a lower one-sided 95% confidence limit of 98.7%). For the inter-lot and inter-site reproducibility of negative samples, as described in the Specificity Study above, 501 assays of 505 sterile LRAP units gave the expected negative result (reproducibility of 99.2%, with a lower one-sided 95% confidence limit of 98.2%). For the inter-lot and inter-site reproducibility testing with the 11-member test panel, the expected BacTx® result was observed with 396 out of 396 samples. No statistically significant difference in reproducibility was observed between the three sites or between the three lots (p=1.0, Fisher-Freeman-Halton test). All 360 bacterial panel members were successfully detected with the BacTx® Assay, as shown in Table 3.4, and all sterile samples were negative in the BacTx® Assay.

TABLE 3.4
Inter-lot/Inter-site Reproducibility Results for the LRAP Study
Table 4. Inter-lot/Inter-site Reproducibility Results for the LRAP Study

| | Logs | | | |
|---|---|---|---|---|
| | Above | Expected | | |
| | Limit of | BacTx ® | # Detected | Detection |
| Sample ID | Detection | Result | (out of 36) | Rate |
| Escherichia coli | 0.5 | FAIL | 36 | 100% |
| Staphylococcus aureus | 0.8 | FAIL | 36 | 100% |
| Bacillus cereus | 1.4 | FAIL | 36 | 100% |
| Staphylococcus | 1.0 | FAIL | 36 | 100% |
| epidermidis | | | | |
| Klebsiella oxytoca | 0.5 | FAIL | 36 | 100% |
| Pseudomonas aeruginosa | 0.8 | FAIL | 36 | 100% |
| Streptococcus agalactiae | 1.3 | FAIL | 36 | 100% |
| Serratia marcescens | 1.1 | FAIL | 36 | 100% |
| Clostridium perfringens | 0.7 | FAIL | 36 | 100% |
| Propionibacterium acnes | 1.2 | FAIL | 36 | 100% |

### LR-WBDP Study Description:

Reproducibility of the BacTx® Assay Kit was determined inter-assay, inter-lot, and inter-site for both negative and positive (spiked) assays. The analytical sensitivity study above served as an inter-assay reproducibility study for spiked pools, as 10/10 positive BacTx® Assay results were required to be positive for the presence of bacteria at a given titer before the assay was validated as positive. For the interassay reproducibility of negative BacTx® assays, 21 unique, sterile 6-unit LRWBDP pools were tested with 3 kit lots at a single site with 10 replicates of the BacTx® Assay for a total of 210 assays. For the inter-lot and inter-site reproducibility, a test panel was used for testing at three sites using three lots of BacTx® Assay Kits on three different days. The test panel consisted of 10 bacterial members and one negative member, and the composition of the positive panel members is listed in Table 3.5. Sterile 6-unit LR-WBDP pools were used for testing, verified sterile by plate culture.

TABLE 3.5
Inter-lot and Inter-site Reproducibility Test Panel for LR-WBDP Study

| | Logs | | | |
|---|---|---|---|---|
| | Above | Expected | | |
| | Limit of | BacTx ® | # Detected | Detection |
| Sample ID | Detection | Result | (out of 36) | Rate |
| Escherichia coli | 1.0 | FAIL | 36 | 100% |
| Staphylococcus aureus | 1.2 | FAIL | 36 | 100% |
| Bacillus cereus | 1.4 | FAIL | 36 | 100% |
| Staphylococcus | 1.0 | FAIL | 36 | 100% |
| epidermidis | | | | |
| Klebsiella oxytoca | 1.2 | FAIL | 36 | 100% |
| Pseudomonas aeruginosa | 0.8 | FAIL | 36 | 100% |
| Streptococcus agalactiae | 1.2 | FAIL | 36 | 100% |
| Serratia marcescens | 1.2 | FAIL | 36 | 100% |
| Clostridium perfringens | 0.8 | FAIL | 36 | 100% |
| Propionibacterium acnes | 1.2 | FAIL | 36 | 100% |

### LR-WBDP Study Results:

For the inter-assay reproducibility of negative assays, 209 out of 210 assays gave the expected negative result (99.5% concordance with a lower one-sided 95% confidence limit of 97.9%). For the inter-lot and inter-site reproducibility of negative samples, as described in the Specificity Study above, 431 assays of 432 sterile, 6-unit platelet pools gave the expected negative result (a specificity of 99.8% with a lower one-sided 95% confidence limit of 99.0%). No statistically significant difference in reproducibility was observed among the three lots (p=1.0, Fisher-Freeman-Halton test) used for specificity testing. For the inter-lot and inter-site reproducibility testing with the 11-member test panel, the expected BacTx® result was observed with 395 out of 396 samples. No statistically significant difference in reproducibility was observed between the three sites or between the three lots (p=1.0, Fisher-Freeman-Halton test). All 360 bacterial panel members were successfully detected with the BacTx® Assay, as shown in Table 3.5.

### Potentially Interfering Substances Study

Turbidity-Causing Substances

### LRAP Study Description:

In the assay detection system, a dedicated photometer is used to monitor the change in absorbance of green colored light that passes through the BacTx® Reaction Tube during the 30 minute assay. Since the determination of a “Fail” or “Pass” result in the BacTx® assay is based on whether the absorbance exceeds 0.5 during the assay, endogenous substances or specific platelet conditions that contribute to sample turbidity may potentially interfere with the BacTx® assay. These include hyperproteinemia, hypergammaglobulinemia, hemolysis, hypercholesterolemia and lipemia. In addition, specific platelet conditions may also interfere with the proper functioning of the Lysis, Extraction, or Neutralization Reagents used during sample preparation. These conditions include high and low pH, platelet concentration, and red blood cell concentration. The concentrations of interfering substance were tested at pathological levels compared to normal (or reference) levels.

To test each of the substances or conditions described above, 100 positive samples (10 samples for each of the 10 bacterial strains, in which the concentration of bacteria was 0.5-1.5 logs above the limit of detection (LOD) determined during the analytical sensitivity study for each strain) and 10 negative samples were prepared using LRAP containing the interfering substance or condition. All of the LRAP units used for this study were 5 days old or less. The concentrations of the ten bacterial strains used for interference testing are the same as for the reproducibility study. They are listed in Table 3.4. Three lots of BacTx® Bacterial Detection Kits were used to prepare and test 10 samples for each bacterial strain listed in Table 3.4, and three lots were also used to test 10 negative samples. For each set of 10 samples, 3 samples were prepared with one lot, another 3 samples with a second lot, and the remaining 4 samples with a third lot. A summary of the sample conditions tested can be found in Table 3.7. The concentrations of interfering substances were tested at pathological levels compared to normal (or reference) levels.

### LR-WBDP Study Description:

To test each of the interfering substances or conditions, a six-unit pool of LR-WBDPs containing the interfering substance was tested using the test panel of bacteria described in Table 3.6. Three kit lots were used to prepare and test the samples by three different users. 10 replicates of each test panel member were tested for each interfering substance. The concentration of the interfering substance was measured in the platelet pool and, if sufficient volume was available, prior to pooling. A summary of the sample conditions tested can be found in Table 3.7.

### LRAP Study Results:

Out of the 1300 positive samples tested with potential interferents, 100% were detected with the BacTx® Assay. Out of the 130 negative samples tested, no false positives were observed. Based on these results, the following substances and platelet conditions do not interfere with the BacTx® Assay: 50-200% normal platelet concentration, low, normal and high pH, 0.7% hematocrit, hemolysis, hyperproteinemia, hypoproteinemia, lipemia, hypercholesterolemia, and hypergammaglobinemia (IgA, IgG, and IgM).

### LR-WBDP Study Results:

Out of the 1300 positive samples tested, 100% were detected with the BacTx® Assay. Out of the 427 negative samples tested, no false positives were observed. Based on these results, the following substances and platelet conditions do not interfere with the BacTx® Assay: 50-200% normal platelet concentration, low and high pH, 0.7% hematocrit, hemolysis, hyperproteinemia, hypoproteinemia, lipemia, hypercholesterolemia, and hypergammaglobinemia (IgA, IgG, and IgM).

TABLE 3.6
Test Panel for LR-WBDP Interfering Substances Study

| | Logs Above |
|---|---|
| Sample | Limit of Detection |
| Escherichia coli | 1.0-1.3 |
| Staphylococcus aureus | 1.1-1.2 |
| Bacillus cereus | 1.4 |
| Staphylococcus epidermidis | 0.6-1.0 |
| Klebsiella oxytoca | 0.9-1.2 |
| Pseudomonas aeruginosa | 0.8-1.1 |
| Streptococcus agalactiae | 1.2-1.5 |
| Serratia marcescens | 0.8-1.2 |
| Clostridium perfringens | 0.5-0.8 |
| Propionibacterium acnes | 1.2-1.3 |

TABLE 3.7
Summary of Interfering Substances Conditions and Normal (Reference) Values

| | Test Concentration | Test Concentration | Normal (Reference) |
|---|---|---|---|
| Condition | for LR-WBDP Study | for LRAP Study | Values |
| Low Platelet Concentration | 50% of normal^{b} | 50% of normal^{a} | 3.0 × 10¹¹ platelets/ |
| | (4.2-6.1) × 10⁸ platelets/mL | (5.0-6.0) × 10⁸ platelets/mL | 250-300 mL for LRAP^{a} |
| High Platelet Concentration | 200% of normal^{b} | 200% of normal^{a} | 5.5 × 10¹³ platelets/ |
| | (1.7-2.4) × 10⁹ platelets/mL | (2.0-2.4) × 10⁹ platelets/mL | 45-65 mL for LR-WBDP^{b} |
| Low pH | 5.5-5.68 | 5.5 | 6.2^{b} |
| Normal pH | 7.15-7.36 | 7.16-7.21 | 7.38-7.42^{c} |
| High pH | 8.4-8.8 | 8.5 | |
| Red Blood Cells | 0.7% hematocrit | 0.7% hematocrit | 0-0.4%^{d} |
| Hemolysis | 0.25 g/dL hemoglobin | 0.25 g/dL hemoglobin | 0-0.13 g/dL^{e} |
| Hyperproteinemia | 10.1-10.7 g/dL | 10.4 g/dL | 6.0-8.3 g/dL^{f} |
| Hypoproteinemia | 2.3-3.3 g/dL | 3.2 g/dL | |
| Lipemia | 206-484 mg/dL | 1350 mg/dL | <150 mg/dL^{g} |
| Hypercholesterolemia | 212-436 mg/dL | 234 mg/dL | <200 mg/dL^{h} |
| Hypergammaglobulinemia (IgG) | 3600-4000 mg/dL | 3500 mg/dL | 560-1800 mg/dLⁱ |
| Hypergammaglobulinemia (IgM) | 730 mg/dL | 1000 mg/dL | 45-250 mg/dLⁱ |
| Hypergammaglobulinemia (IgA) | 1675 mg/dL | 2205 mg/dL | 100-400 mg/dLⁱ |

^{a}December 2007. “Guidance for Industry and FDA Review Staff: Collection of Platelets by Automated Methods” Rockville, MD: City, State: U.S. Department of Health and Human Services, Food and Drug Administration, Center for Biologics Evaluation and Research. (Downloaded on Jun. 22, 2011).
^{b}21 CFR, Chap 1, Subpart C, section 640.24 (4-1-12 Edition).
^{c}Normal range of blood serum from “Merck Manual of Diagnosis and Therapy, Section: Endocrine and Metabolic Disorders, Subject: Acid Base Regulation and Disorders. Topic: Introduction” http://www.merckmanuals.com/. James L. Lewis, III, MD (ed). July 2008. Merck Sharp & Dohme Corp. Accessed Jun. 22, 2011 <http://www.merckmanuals.com/professional/sec12/ch157/ch157b.html>.
^{d}Upper limit based on 2 mL of packed red blood cells in 500 mL plasma volume, as described in (b).
^{e}Upper limit based on complete hemolysis of 0.4% hematocrit.
^{f}Based on normal serum total protein level from: David C. Dugdale. “Total Protein”. http://www.nlm.nih.gov/medlineplus/medlineplus.html. David Zieve (Rev). May 2009. U.S. National Library of Medicine, U.S. Department of Health and Human Services, National Institutes of Health. Accessed Jun. 22, 2011 <http://www.nlm.nih.gov/medlineplus/ency/article/003483.htm>.
^{g}Based on serum triglyceride reference range classified as desirable from “Triglycerides”. http://www.labtestsonline.org. March 2011. American Association for Clinical Chemistry. Accessed Jun. 22, 2011 <http://www.labtestsonline.org/understanding/analytes/triglycerides/sample.html>.
^{h}Based on serum cholesterol reference range classified as desirable from “Cholesterol”. http://www.labtestsonline.org. March 2011. American Association for Clinical Chemistry. Accessed Jun. 22, 2011 <http://www.labtestsonline.org/understanding/analytes/cholesterol/test.html>.
ⁱBased on normal serum levels from: David C. Dugdale. “Quantitative Nephelometry”. http://www.nlm.nih.gov/medlineplus/medlineplus.html. David Zieve (Rev). June 2010. U.S. National Library of Medicine, U.S. Department of Health and Human Services, National Institutes of Health. Accessed Jun. 22, 2011 <http://www.nlm.nih.gov/medlineplus/ency/article/003545.htm>.

### LR-WBDP Study of Immunoassay Interferents:

### Description:

The BacTx® Assay is an enzyme-based assay and is technologically different than immunoassays such as ELISA or lateral-flow. Peptidoglycan in the prepared platelet sample is bound by a peptidoglycan recognition protein, which has a peptidoglycan binding domain that is similar in structure to T7 lysozyme. In contrast, lateral flow immunoassays typically use mono- and polyclonal antibodies from multiple sources (mouse, rabbit, goat) for both analyte capture and detection. Interference of immunoassays caused by endogenous substances is well-documented (Tate J et al., Interferences in immunoassay, Clin Biochem Rev. (2004) 25(2):105-120; Dimeski G., Interference testing.” Clin Biochem Rev. (2008) 29 Suppl 1:S43-48). Substances that interfere with antibody binding are heterophilic antibodies, autoimmune antibodies (such as rheumatoid factor and antinuclear antibodies), and human anti-animal antibodies. To demonstrate that the BacTx® Assay is not affected by these common immunoassay interferents, pooled platelets were resuspended in patient plasma or serum containing heterophilic antibodies, autoimmune antibodies, and human anti-mouse antibody (HAMA) and tested with the BacTx® Assay. The platelet samples containing the immunoassay interferent were tested using the test panel described in Table 3.6. Three kit lots were used to prepare and test the samples. 10 replicates of each test panel member were tested for each interfering substance. The immunoassay interferents tested are described in Table 3.8.

TABLE 3.8
Samples for Immunoassay Interference Testing in LR-WBDP Study

| Sample | Concentration |
|---|---|
| Heterophilic plasma | Positive heterophile antibody |
| | test |
| Autoimmune Antibodies | ANA (Positive, qualitative test) |
| | dsDNA (10.4-123 IU/mL) |
| | RF (67-1075 IU/mL) |
| Human anti-mouse antibody (HAMA) | 37-329 ng/mL |

### LR-WBDP Study Results:

Out of the 370 positive samples tested, all 370 were successfully detected with the BacTx® Assay. Out of the 91 negative samples tested, all 91 tested negative in the BacTx® Assay. Based on these results, the Immunoassay interference substances tested in this study do not interfere with the BacTx® Assay.

### LR-WBDP Study of Prozone (Hook Effect)

### Description:

The hook effect is a type of assay interference most commonly associated with immunoassays, in which the concentrations of antigen are in such excess that the capture antibodies and labeled antibodies do not simultaneously bind the same analyte unit and leads to falsely negative test results. While the BacTx® Assay is not an immunoassay, testing was performed to determine if a hook effect is present at high concentrations of bacteria present in platelet samples. Single LR-WBDP units (3-days-old) were inoculated with moderate concentrations (103-105 CFU/mL) of each of the eight aerobic bacterial strains tested in the analytical sensitivity study. The platelet units were incubated for five days to allow the bacteria to proliferate and reach stationary growth phase. Aliquots were withdrawn at 3, 4, and 5 days after inoculation for dilution plating on 5% sheep blood agar plates to determine if the bacteria in the unit had reached stationary phase. On the fifth day after inoculation, a platelet pool was made using the inoculated unit and 5 in-date, sterile (as determined by agar plating), BacTx-unreactive, LR-WBDP units. For each of the three BacTx Test lots, ten samples from the platelet pool were tested in the BacTx Test to determine if any falsely negative test results occur. Dilution plating on 5% sheep blood agar plates was performed on the platelet pool to determine final bacterial concentration in the pool. Since the two anaerobes (Clostridium perfringens and Propionibacterium acnes) do not grow to high concentrations in platelet concentrates, 4 mL volumes of high titer cultures of each strain were centrifuged and the bacteria pellets were resuspended in separate 4 mL volumes of platelets from single in-date, sterile, WBDP units. For each strain, a six-unit platelet pool was made by combining the bacterially contaminated platelets with platelets from 5 in-date, sterile (as determined by agar plating), BacTx® unreactive, LR-WBDP units (a 1:5 volume ratio). For each of the three kit lots, ten replicates from the platelet pool were tested in the BacTx® Assay to determine if any falsely negative test results occur. Dilution plating on anaerobic culture plates was performed on the platelet pool to determine the final bacterial concentration in the pool.

### Results:

Table 3.9 shows the growth of bacteria in LR-WBDPs, and the results of the Prozone testing with the BacTx® Assay. The bacteria titers recorded over the 5 day period for the aerobes indicated that the eight aerobes had either attained stationary phase growth or a very high titer (>1E9 CFU/mL) by day 5. A greater than expected decrease in the bacterial concentration was observed upon pooling of the bacteria-containing unit with the 5, in-date LR-WBDP units for almost all of the aerobes tested. This loss of viability was attributed to the susceptibility of the stationary phase bacteria to the bactericidal properties of in-date LR-WBDP platelets upon pooling. For each of the ten strains, none of the 30 samples tested with the BacTx® Assay were falsely negative. These results indicate that the BacTx® Assay is not affected by Prozone effects caused by high bacterial concentrations attainable in LR-WBDP pools.

TABLE 3.9
Prozone Testing Results for the LR-WBDP Study

| | | | | | | | Samples |
|---|---|---|---|---|---|---|---|
| | | Bacterial Concentration (CFU/mL) | | | | | Detected |
| | | Individual Inoculated WBDP Unit | | | | DAY 5 | by BacTx ® |
| BACTERIA | | DAY 0 | DAY 3 | DAY 4 | DAY 5 | (6-unit pool) | (out of 30) |
| Aerobe | Bacillus cereus | 2.1 × 10⁵ | n.d. | 2.6 × 10⁷ | 3.7 × 10⁷ | 9.0 × 10⁵ | 30 |
| | Escherichia coli | 1.3 × 10⁵ | 4.7 × 10⁸ | 4.4 × 10⁸ | 4.5 × 10⁸ | 1.1 × 10⁸ | 30 |
| | Klebsiella oxytoca | 7.3 × 10⁴ | 3.9 × 10⁸ | 1.0 × 10⁹ | 1.3 × 10⁹ | 1.6 × 10⁸ | 30 |
| | Pseudomonas aeruginosa | 3.0 × 10⁵ | 3.0 × 10⁹ | 3.0 × 10¹⁰ | 3.3 × 10⁹ | 2.2 × 10⁸ | 30 |
| | Streptococcus agalactiae | 4.0 × 10⁵ | n.d. | 2.4 × 10⁷ | 1.2 × 10⁸ | 3.9 × 10⁶ | 30 |
| | Staphylococcus aureus | 2.6 × 10⁵ | n.d. | 5.6 × 10⁸ | 5.0 × 10⁹ | 2.0 × 10⁸ | 30 |
| | Staphylococcus epidermidis | 5.0 × 10⁵ | 1.7 × 10⁹ | 2.6 × 10⁹ | 1.0 × 10⁹ | 3.7 × 10⁷ | 30 |
| | Serratia marcescens | 3.1 × 10⁴ | 8.0 × 10⁸ | 4.6 × 10⁹ | 3.7 × 10⁹ | 4.2 × 10⁸ | 30 |
| Anaerobe | Clostridium perfringens | | | | | 1.1 × 10⁷ | 30 |
| | Propionibacterium acnes | | | | | 3.4 × 10⁸ | 30 |

n.d. = not determined.

### Example 4

### Comparative Clinical Testing Results of Assay Methods

The clinical data provided herein reflects use of the BacTx® Bacterial Detection Kit for the detection of bacteria in Leukocyte Reduced Apheresis Platelets (LRAPs). BacT/ALERT® and the BacTx® were used as the predicate devices in these studies. The sensitivity, specificity, and time to detection were determined at three sites. Use of the assay detection system of the present invention is expected to produce substantially the same sensitivity, reproducibility, accuracy, and functionality as the data presented below.

### Procedures and Results:

### Analytical Sensitivity Testing:

### Overview:

As in Example 3 for Leukocyte Reduced Apheresis Platelets, sensitivity was determined in aliquots of LRAPs spiked with bacteria. The analytical sensitivity was determined for 10 bacterial species at two external sites. At each site, LRAPs were tested with bacterial titers between 1×10³ CFU/mL and 8×10⁴ CFU/ml. Target titers for testing are shown in Table 4.1, which also shows the spiking strategy. Ten BacTx® Assays were performed for each individual titer tested. Testing was initiated with the most dilute titer, and was continued with increasingly higher bacterial concentrations until 1 0 out of 10 BacTx® Assays were positive. Once 10/10 positive BacTx Assays were obtained for a given species, testing was stopped for that species, and higher titers were not tested. The concentration at which 10/10 BacTx® Assays were positive was taken to be the analytical sensitivity of the species. Analytical sensitivity results were compared between sites, and the analytical sensitivity claim made for the assay is based on the higher titer obtained between the sites. Sterility of the LRAP units used in analytical sensitivity training was established using BacT/ALERT® culture testing (BPA and BPN bottles.) Sensitivity data was collected only from LRAPs that were shown to be sterile by BacT/ALERT® culture. The bacterial titers of the cultures used for spiking were estimated based on Optical Density (OD) readings of bacteria cultures. The actual titers of the spiked mini-platelet pools were determined by quantitative agar plating.

### Methods:

The sensitivity of the BacTx® Assay was tested in aliquots of LRAPs. The FDA has authorized use of spiked aliquots for this testing. The bacterial strains tested are shown in Table 4.1.

TABLE 4.1
Bacterial Species for Clinical Performance Testing

| Species | ATCC Number |
|---|---|
| Gram Positives: | |
| Staphylococcus aureus | 27217 |
| Staphylococcus epidermidis | 49134 |
| Bacillus cereus | 11778 |
| Streptococcus agalactiae | 12386 |
| Gram Negatives: | |
| Serratia marcescens | 43862 |
| Pseudomonas aeruginosa | 27853 |
| Escherichia coli | 25922 |
| Klebsiella oxytoca | 43863 |
| Anaerobes: | |
| Clostridium perfringens | 3629 |
| Propionibacterium acnes | 11827 |

The spiking strategy is shown in Table 4.2. Each bacterial strain was diluted to the appropriate titer, and a 0.6 mL volume was spiked into a 12 mL aliquot from one LRAP unit. After spiking, the estimated bacterial titers in the 12 mL aliquot of LRAP were: 1×10³ CFU/mL, 5×10³ CFU/mL, 1×10⁴ CFU/mL, 2×10⁴ CFU/mL, 4×10⁴ CFU/mL, and 8×10⁴ CFU/ml. To verify that sterile and BacTx®—negative LRAPs were used for pooling, an aliquot was removed for BacT/ALERT® and BacTx® testing before spiking Three BacTx®—kit lots were used for each bacterial strain, Lots A, Band C. A BacTx®—assay was performed with unspiked LRAP for each kit lot, and the result on unspiked LRAPs had to be negative before testing was initiated with an aliquot of spiked LRAP. During testing of spiked LRAPs, a total of 10 BacTx®—assays were tested at each bacterial concentration, 4 BacTx®—assays using Lot A, 3 BacTx®—assays using Lot Band 3 BacTx®—assays using Lot C. Testing was initiated at the lowest bacterial concentration, and testing was stopped once 10 out of 10 replicates were positive for a given titer of bacteria. The actual bacterial concentration in each of the spiked LRAP aliquots was determined by quantitative culture on Blood Trypticase Soy Agar (TSA Blood Agar) plates.

TABLE 4.2
Spiking Protocol for Analytical Sensitivity Study

| | Inoculum Concentration | Estimated conc. in 12 mL |
|---|---|---|
| Order of Testing | in 0.6 mL volume | LRAP aliquot |
| 1^{st} | 2 × 10⁴ CFU/mL | 1 × 10³ CFU/mL |
| 2^{nd} (if necessary) | 1 × 10⁵ CFU/mL | 5 × 10³ CFU/mL |
| 3^{rd} (if necessary) | 2 × 10⁵ CFU/mL | 1 × 10⁴ CFU/mL |
| 4^{th} (if necessary) | 4 × 10⁵ CFU/mL | 2 × 10⁴ CFU/mL |
| 5^{th} (if necessary) | 8 × 10⁵ CFU/mL | 4 × 10⁴ CFU/mL |
| 6^{th} (if necessary) | 1.6 × 10⁵ CFU/mL | 8 × 10⁴ CFU/mL |
| Total # BacTx ® | Per species | 13-63 |
| Assays | All species | 130-630 |

For spiking, bacterial cultures were inoculated using 3-5 colonies from a freshly streaked (overnight) agar plate, and were grown up in appropriate (species-specific) growth medium in a microbiology shaker/incubator for a 2-4 hour period. The estimated titer of bacteria in the culture was determined by measuring Optical Density (OD) at 600 nm using predefined growth curves data. The linear regressions correlate optical density to bacterial titer in the culture. Since measurement of culture turbidity to determine bacterial concentration is an indirect and imprecise method, the quantity of bacteria spiked generated by the growth curves serves only as an estimate. The actual titer of bacteria in the platelet pool was determined by quantitative plating (in triplicate) on blood TSA agar plates (or other growth media, as appropriate for a given species). The estimated titer is usually accurate to within 2-4 fold of the actual titer. However, it is possible that in certain experiments, the actual titer will significantly deviate from the estimated titers. If the estimated titer was inaccurate, leading to a large gap between two consecutive actual titers tested, clinical sites were instructed to repeat the study to obtain performance data within the titer range missed in the initial study.

### 5.2 Results of Analytical Sensitivity Testing:

### Discussion:

The analytical sensitivity limit of detection determined at both sites, and the final claimed analytical sensitivity, are shown in Table 4.3.

TABLE 4.3
Analytical Sensitivity of the BacTx ® Assay (CFU/mL)

| | Site 1 | Site 2 | |
|---|---|---|---|
| | (Long Island) | (Cleveland) | |
| Species | Sensitivity | Sensitivity | Overall |
| Escherichia coli | 7.0 × 10⁴ | 3.3 × 10⁴ | 7.6 × 10⁴ |
| Pseudomonas aeruginosa | 2.7 × 10⁴ | 2.0 × 10⁴ | 2.7 × 10⁴ |
| Klebsiella oxytoca | 1.8 × 10⁴ | 7.3 × 10³ | 1.8 × 10⁴ |
| Serratia marcescens | 4.2 × 10³ | 5.3 × 10³ | 5.3 × 10³ |
| Propionibacterium acnes | 5.0 × 10³ | 8.5 × 10³ | 8.5 × 10³ |
| Staphylococcus aureus | 2.2 × 10³ | 1.1 × 10³ | 2.2 × 10³ |
| Staphylococcus epidermidis | 1.3 × 10³ | 6.3 × 10² | 1.3 × 10³ |
| Streptococcus agalactiae | 4.5 × 10³ | 3.3 × 10³ | 4.5 × 10³ |
| Clostridium perfringens | 9.4 × 10² | 4.8 × 10³ | 4.8 × 10³ |
| Bacillus cereus | 1.4 × 10³ | 1.9 × 10³ | 1.9 × 10³ |

In order to collect the complete data set, it was necessary to repeat the analytical sensitivity studies for two organisms:

1) Escherichia coli: Analytical sensitivity testing at Site 1 was repeated for E. coli because the highest titer tested on the first day of testing turned out to be of 2.7×10⁴ CFU/mL, which was too low of a titer for 10/10 BacTx® Assays to be positive for the presence of bacteria. The analytical sensitivity study for E. coli was repeated, and on the second day 10/10 assays were positive for the presence of E. coli at a titer of 7.6×10⁴ CFU/mL, which is the limit of detection reported for Site 1 in Table 4.3 for this species.

2) Proprionibacterilim acnes: Analytical sensitivity testing at Site 1 was repeated for Piopribnioacteriutn acnes because the culture was grown over the weekend, instead of overnight, and had reached the stationary phase. The testing protocol specifies that cultures for spiking must be in mid-log phase. The testing was repeated with an overnight culture in log phase, and an analytical sensitivity of 5.0×10 CFU/mL was observed, which is the limit of detection reported for Site 1 in Table 4.3 for this species.

### Conclusion:

The highest analytical sensitivity with LRAPs was observed for S. epidermidis at 1.3×10³ CFU/mL. The BacTx® Assay was least sensitive for E. coli at 7.6×10⁴ CFU/mL. Titers determined between sites varied by approximately 5-fold for C. perfringens. Titers between sites were virtually identical to each other of P. aeruginosa, S. marcescens, P. acnes and S. agalactiae. Titers were within 2-3-fold of each other between the two clinical sites for all other species.

### Time to Detection Testing:

### Overview:

The Time to Detection study was performed as in Example 3 for Leukocyte-Reduced Apheresis Platelets.” A bacterial growth study was performed in LRAPs to determine the earliest sampling time that the BacTx® Assay could successfully detect bacteria that were inoculated at very low titers (1.3-5.3 CFU/mL) and allowed to proliferate in LRAPs.

For each of the 10 bacterial species listed in Table 4.1, a total of 5 LRAP units were obtained. One of these LRAPs was to serve as a negative control and be spiked only with sterile saline. To insure that bacterial growth in platelets is successfully established for each strain, the other 4 LRAPs were inoculated with bacteria at a titer of 2-5 CFU/ml. Before any spiking was performed, aliquots from all 5 LRAPs was removed for BacT/ALERT® testing to establish the sterility of the units (BPA and BPN bottles), and all 5 LRAPs were tested by BacTx® Assay to establish that the units were not BacTx® Assay-reactive. The identity of the bacteria spiked into LRAPs at the beginning of the study for each strain, and the identity of the bacteria that grew up in the LRAP unit and was present at Day 7 (162-174 hours post-inoculation), was confirmed by the Clinical Microbiology Laboratories at both clinical sites. In all cases, for all strains, the proper strain was identified at inoculation and on day 7, except for the anaerobes, which did not grow in LRAPs, and were not identified on Day 7. Additionally, BacT/ALERT® testing was performed on Day 0, Day 1, Day 2, Day 3 (if necessary) and Day 7. Platelet samples were blinded prior to sample preparation. At each time point when 10 BacTx® Assays were to be performed on spiked LRAP units, three negative (unspiked, sterile) LRAP samples were prepared at the same time. The technician performing the assays was blinded as to which samples were spiked with bacteria and which were sterile.

### Method:

For each strain, 1 LRAP was spiked with sterile saline, 2 LRAPs were spiked at a titer of 2 CFU/mL, and two LRAPs were spiked at a titer of 5 CFU/mL, as measured and calculated by optical density readings using a spectrophotometer. Additionally, an aliquot was removed from the diluted cultures prior to spiking for quantitative plate culturing, so that the actual concentration of bacteria spiked into the LRAPs could be determined. On day 1 (22-26 hours post inoculation), sites were instructed to count the colonies from the quantitative plating, select two of the four spiked LRAPs for further analysis, and discard the other two LRAPs. The clinical sites were instructed to select LRAPs that were confirmed to be inoculated at a titer between 2-5 CFU/ml. The two units with the highest inoculated titer between 2-5 CFU/mL were selected to continue the study. If it was found that there were not two LRAPs inoculated within the range of 2-5 CFU/mL, sites were instructed to consult with manufacturer for selection of appropriate LRAP units. In four instances, LRAPs that were inoculated with titers between 1-2 CFU/mL were used for the growl h study. In only one instance was an LRAP used that was inoculated at a concentration higher than 5 CFU/mL used (B. cereus at 5.3 CFU/mL at Site 1.) See Table 4.4 for the actual titers spiked into LRAP units.

The LRAP unit inoculated with saline had to be negative for bacterial growth by quantitative plate culture in order for the study to continue for a given species. Also, on Day 1 (22-26 hours post-inoculation), an aliquot was removed from the saline inoculated LRAP and the two bacterially-inoculated LRAPs that were selected by the criteria described in the paragraph above, and the aliquots were subjected to quantitative plate culturing. On Day 2 (44-52 hours post-inoculation), the quantitative plates from Day 1 were counted, and the LRAP unit that best supported bacterial growth was selected for BacTx® Assay. Ten BacTx® Assay were performed on the selected LRAP unit with samples blinded as described above.

If 10/10 BacT X® assays were positive, the Time to Detection for the bacterial species was determined to be 48 hours, and BacTx® Assay was not performed at 72 hours. At each time point when 10 BacTx® Assays were performed, three kit lots were used (4 samples from one lot, 3 samples from the other two lots.) Similarly, the same three kit lots were used for the blinded negative samples that were run in parallel, one kit lot per negative sample. If 10/10 BacTx® Assays were NOT positive on Day 2, the testing was repeated on Day 3 (66-78 hours post-inoculation.) If 10/10 BacT X® assays were positive on Day 3, the Time to Detection for the bacterial species was determined to be 72 hours. In all cases, the spiked LRAP unit was tested on Day 7 (162-174 hours post-inoculation.) On any day in which BacT X® assays were-performed (Day 2, Day 3 and Glay 7), aliquots were removed from the LRAPs for quantitative plate counting so that the titer of bacteria present in the LRAP at time of BacT X® assay could be determined. As mentioned above, BacT/ALERT® testing on the inoculated LRAPs was also conducted on Day 0, Day 1, Day 2, Day 3 (if necessary—only performed if BacT X® assays were required to be run) and Day 7. In addition to testing of the 8 aerobic strains, the FDA requested that we attempt to grow the anaerobic strains (P. acnes and C. perfringens) in this manner, and prove that we could not grow them in LRAP units.

### Results of Time to Detection Testing:

### Discussion:

A summary of the BacTx® Assays, BacT/ALERT®, and quantitative plate culture results for the Time to Detection study performed at both sites is shown in Table 4.4. For each bacterial strain tested at each site, the earliest time point at which 10 out of 10 BacTx® Assays were positive is shaded in grey. Of the 8 aerobes tested, six species were detected at 48 hours. S. agalactiae was detected at 48 hours at one site and at 72 hours at the second site. P. aeruginosa was detected at 72 hours at both sites. As anticipated, we could not detect the presence of colonies on quantitative plate cultures in LRAPs inoculated with anaerobes, and BacTx® Assays were negative for LRAP inoculated with the anaerobic species.

### Conclusion:

Based on these results, the optimal time for detection of all of the bacterial strains that proliferate in platelet units is 72 hours. For each bacterial strain tested at both sites, the ability of the BacTx® Assay to detect bacteria in 10 out of 10 samples is supported by the plate culture results. For the aerobic bacteria, at each time point that 10 out of 10 samples were detected in the BacTx® Assay, the results of BacT/ALERT® culture testing were also positive for one or both types of bottles. For anaerobic bacteria, C. perfringens did not grow in platelets during the time to detection study and were not detected by BacTx® Assay, BacT/ALERT® or agar plate culture; P. acnes was not detected by the BacTx® Assay or by agar plating, and was also not detected by BAcT/ALERT® within the normal 5 day shelf life of platelets. The data indicates that BacTx® Assay performance in LRAPs is substantially equivalent to BK11 0054 and the BacTx® Assay yields equivalent results to automated culture methods.

TABLE 4.4
Summary of BacTx ® testing, BacT/ALERT ® , and QPC Results for the Time-to-Detection Study

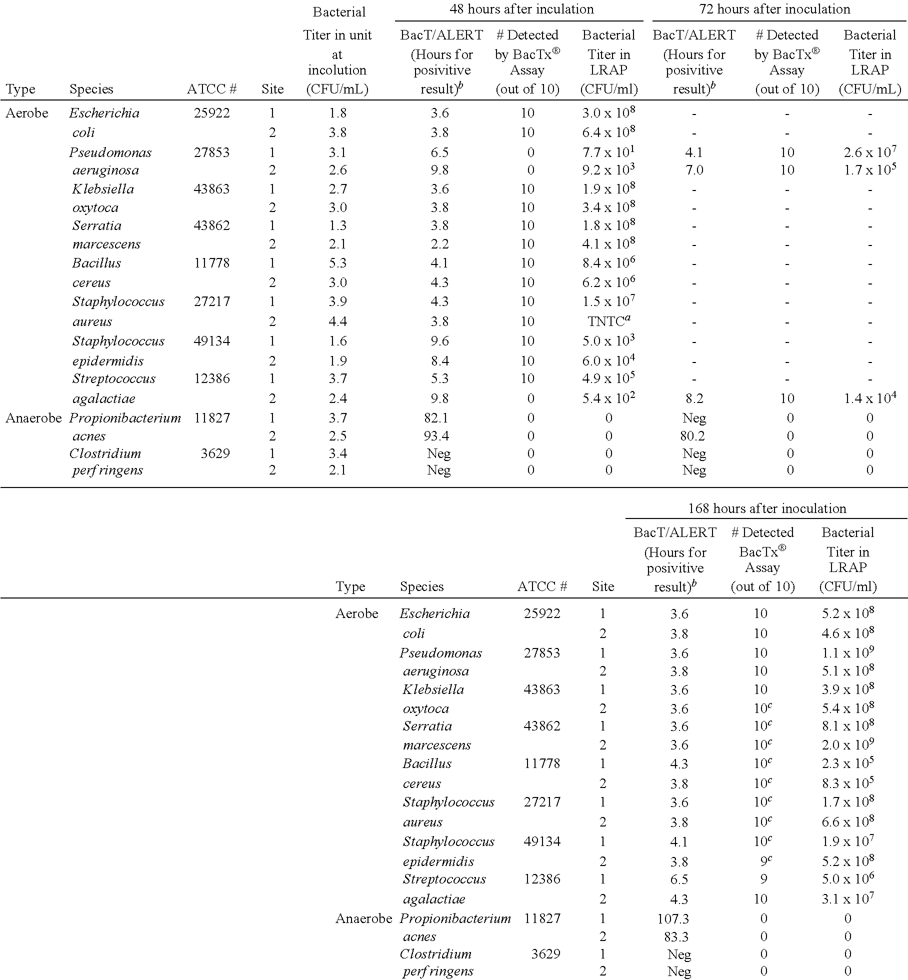

aTNTC = Top numerous to count after dilution plating.
bEarliest positive among BPN and BPA bottle pair (Neg = negative for 7 days).
cOne or more processed samples gave an abort result, due to high sample tubidity, 11-fold of the processed sample tested, as described in CD12004.

### Specificity Testing:

### Overview:

The Specificity study was performed as in Example 3 for Leukoreduced Apheresis Platelets.” Specificity of the BacTx® Assay on LRAPs was assessed with 6 lots of BacTx® Bacterial Detection Kits on 505 unique LRAP units at two external clinical sites.

### Methods:

Specificity of the BacTx® Assay for LRAPs was determined using 505 unique LRAP units and 6 BacTx® Kit lots. The specificity study was split between both external clinical study sites. At Site 2 409 LRAPs were tested, and 96 LRAPs were tested at Site 1. A two-tier testing algorithm was used. If an LRAP unit was found to be initially reactive, it was retested with two BacTx® Assays. If either of the retests were positive, the unit was considered “Repeat Reactive.” If both of the retests were negative in the BacTx® Assay, the unit was considered nonreactive. Users of the BacTx® Assay will be directed in the Instructions for Use to use this testing algorithm, and will be instructed that LRAPs that test negative in both retests may be considered negative for the presence of bacteria. The sterility of each LRAP unit tested during the study was established using blood agar plates.

### Results of Specificity Testing:

### Discussion:

A total of 505 unique sterile mini-pools were tested, of which 501 were negative for the presence of bacteria in the BacTx® Assay (BacTx® Assay result=PASS.) Of the 4 LRAP units that were positive in initial testing (0.79%), 3 were negative during retest. Thus 1 sample out of 505 was Repeat Reactive in the assay. This corresponds to a specificity, defined as (1—the frequency of Repeat Reactive samples) of 99.8%, with a lower one-sided 95% confidence limit of 99.1%.

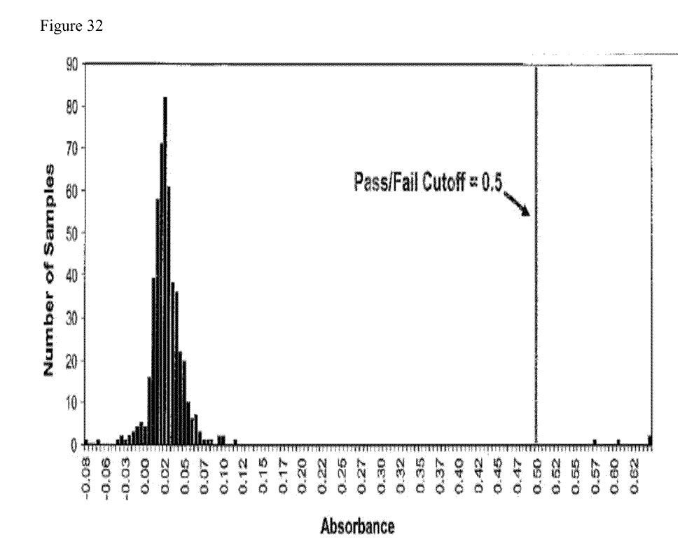

The BacTx® Assay measures the change in absorbance over a 30 minute period. The mean change in absorbance at the end of the 30 minute assay period for the 501 BacTx®—negative assays was 0.019, with a standard deviation of 0.020. The absorbance change threshold between a Pass and Fail result has been set at 0.500 (FIG. 32). The BacTx® Assay is not a quantitative assay. Once the activating threshold of peptidoglycan is detected, the prophenoloxidase cascade is activated and exponentially increasing amounts of phenoloxidase are created. The dramatic increase in phenoloxidase rapidly results in complete enzymatic conversion of the phenolic substrate to the colored product. Therefore, at the end of the 30 minute assay, the absorbance change of BacTx®—positive samples is not proportional to the input concentrations of peptidoglycan. Given the all-or-nothing nature of the assay system, the cutoff of the assay has been set as far from the mean negative absorbance as practical, in order to minimize the frequency of false positives. For LRAPs, the assay cutoff is 24 standard deviations from the mean absorbance change observed in the 501 BacTx® Assays of sterile units. Table 4.5 shows a breakdown of BacTx® Assay specificity testing at both clinical sites. No Repeat Reactive false positive LRAP units were observed at Site 2. One Repeat Reactive unit was observed in at Site 1.

TABLE 4.5
Assay Specificity at Clinical Sites

| | Site 1 | Site 2 | |
|---|---|---|---|
| | (Long Island) | (Cleveland) | Total |
| # of Assays | 96 | 409 | 505 |
| # Initially Reaction | 1 | 3 | 4 |
| # Repeat Reaction | 1 | 0 | 1 |
| % Specificity | 99.0% | 100% | 99.8% |
| Mean Absorbance Change | 0.018 | 0.019 | 0.019 |
| Standard Deviation | 0.032 | 0.016 | 0.020 |

### Conclusion:

Using the initially reactive results the BacTx® Assay has a specificity of 99.2% with LRAP units, which is similar to the specificity reported for LR-WBDPs of 99.8%. A comparison of these two results using Fisher's exact test has a p-value of 0.3813. One sample out of 505 was Repeat Reactive in the assay. This corresponds to a specificity, defined as (1—the frequency of Repeat Reactive samples) of 99.8%, with a lower one-sided 95% confidence limit of 99.1%. The selected assay cut-off is 24 standard deviations from the mean change in absorbance observed in the BacTx® Assays of sterile LRAPs tested in this study.

### Reproducibility Testing:

### Overview:

Reproducibility testing was performed as in Example 3 for Leukocyte Reduced Apheresis Platelet Samples.” Reproducibility of the BacTx® Assay kit was assessed inter-assay, inter-site and inter-lot, for both negative and positive samples. Inter-assay reproducibility of positive samples was assessed at external sites during the analytical sensitivity segment of the study, where 10 out of 10 BacTx® Assay results had to be positive before a claim for sensitivity at a given bacterial titer was made. For each bacterial species, at each site, 10/10 BacTx® Assay results were positive for a given titer. A total of 200 positive assays was required for this study (100 positive assays per external site.)

Inter-assay reproducibility of negative samples was assessed. Twenty-one unique, sterile LRAP units were obtained, and 10 platelet samples were prepared from each LRAP unit and tested using three lots of BacTx® Kits, for a total of 210 negative assays.

Inter-lot and inter-site reproducibility was assessed using the Reproducibility Test Panel described in Table 4.6 below. The panel consists of frozen bacterial pellets. There are 10 bacterial species and one negative control tube in each panel, for a total of 11 assays per panel. Three sites performed this testing. Each site conducted three days of testing, and tested three different lots of BacTx® Assay kits. Table 10 shows the schedule of lot testing at each site. On each day of testing, the Reproducibility Test panel was run twice for each lot tested that day (2 lots per day). Thus each day, 22 tests of each lot were performed, a total of 44 BacTx® Assays per day. A total of 132 BacTx® Assays were performed per site, and a total of 132 BacTx® Assays were performed per lot (44 BacTx® Assays per lot per site.) In this study 396 total BacTx® Assays were performed. A tube-to-tube repeatability analysis was also performed on the Time To Fail (TTF) values from the Inter-lot, intersite reproducibility study.

### Methods:

Inter-Assay Testing:

Inter-assay reproducibility of spiked units was evaluated based on results of analytical sensitivity testing of replicates (10 per spiked LRAP unit) as described in the analytical sensitivity study. For reproducibility of negative samples, 10 replicates were sterilely removed from sterile LRAP units. Twenty-one unique LRAPs were tested, and the sterility of the mini-pools was established by removing an additional 1 ml volume from the mini-pool for plating on blood agar. A total of 210 assays were performed.

Inter-Lot and Inter-Site:

The reproducibility study protocol for LRAPs is similar in design to that used for pooled LR-WBDP (BK110054). An eleven member bacteria panel consisting of the ten bacterial strains and a negative member of sterile was prepared, with the bacteria titer of each member between 0.5-1.5 logs of the limit of detection determined for LRAP in the Analytical Sensitivity Study once suspended in 1 ml of LRAP. The composition of the Reproducibility Panel is shown in Table 9. The panels were sent on dry ice to the external clinical sites, and testing was conducted at three sites (External Site 1 and Site 2, and also at lmmunetics) with one user per site. At each site, testing took place on three different days. For each day of testing, two kit lots were tested with one unique LRAP unit, as shown in Table 4.8 below. Each panel member was tested in 36 assays among the three sites. A total of 396 assays were performed. Sterility of the LRAP units used was verified by BacT/ALERT® at external Sites 1 and 2, and by blood agar plating at lmmunetics. The percent agreement with the expected result (binary outcome) along with 2-sided 95% confidence intervals calculated using the score method were determined for each kit lot and site

TABLE 4.6
Bacterial Test Panel for Reproducibility and Bench Studies

| | | Titer after | | |
|---|---|---|---|---|
| | | addition of | Logs above | |
| | | 1 mL | LoD | Expected |
| Panel | | of LRAP | (in 1 mL | BacTx ® |
| # | Species | (cfu/mL) | platelets) | Result |
| 1 | Bacillus cereus | 5.0 × 10⁴ | 1.4 | Fail |
| 2 | Clostridium perfringens | 2.6 × 10⁴ | 0.7 | Fail |
| 3 | Escherichia coli | 2.7 × 10⁵ | 0.5 | Fail |
| 4 | Klebsiella oxytoca | 5.5 × 10⁴ | 0.5 | Fail |
| 5 | Propionibacterium acnes | 1.3 × 10⁵ | 1.2 | Fail |
| 6 | Pseudomonas aeruginosa | 1.7 × 10⁵ | 0.8 | Fail |
| 7 | Serratia marcescens | 7.0 × 10⁴ | 1.1 | Fail |
| 8 | Staphylococcus aureus | 1.3 × 10⁴ | 0.8 | Fail |
| 9 | Staphylococcus epidermidis | 1.3 × 10⁴ | 1.0 | Fail |
| 10 | Streptococcus agalactiae | 1.0 × 10⁵ | 1.3 | Fail |
| 11 | Negative (sterile P85) | — | — | Pass |

Reproducibility testing of negative samples was conducted as part of the Specificity Study. Six kit lots were tested in a total of 505 negative assays.

### Results of Reproducibility Testing:

### Discussion:

### Inter-Lot and Inter-Site Reproducibility

Inter-lot and Inter-assay reproducibility of spiked samples was conducted using the 11 member reproducibility test panel of 10 frozen bacterial pellets and one negative sample. In total, 396 BacTx® Assays were performed, with all 396 giving the expected result, a concordance of 100% between the actual and expected results (see Table 4.7)

TABLE 4.7
Inter-Site Reproducibility of the BacTx ® Assay with the Test Panel

| | Site 1 | Site 2 | Site 3 |
|---|---|---|---|
| | (Long Island) | (Cleveland) | (Immunotics) |
| Total # of BacTx ® | 132 | 132 | 132 |
| Assays | | | |
| # of Assays Concondant | 132 | 132 | 132 |
| with Expected Result | | | |
| % Concondance with | 100% | 100% | 100% |
| Expected Result | | | |
| 2-sided 95% score | 97.2-100.0% | 97.2-100% | 97.2-100% |
| confidence intervals | | | |

Inter-site reproducibility of negative samples was determined during the Specificity Study. 505 sterile, unique LRAP units were tested with a total of 6 kit lots, of which 501 were not positive for the presence of bacteria in the BacTx® Assay. This corresponds to an overall reproducibility of 99.2%, with 2-sided 95% confidence intervals of 98.0%-99.7%. The lower one-sided 95% confidence limit is 98.2%. Inter-site reproducibility is shown in Table 4.8.

TABLE 4.8
BacTx ® Negative Assay Reproducibility at External Sites

| | | Total | |
|---|---|---|---|
| | # of Negative | Assays | Percent Negative |
| Site | BacTx ® Assays | Run | (2-sided 95% CI) |
| Site 1 (Long Island) | 95 | 96 | 99.0% (94.3%, 99.8%) |
| Site 2 (Cleveland) | 406 | 409 | 99.3% (97.9%, 99.8%) |
| Total | 501 | 505 | 99.2% (88.0%, 99.7%) |

### Time to Fail Reproducibility Analysis

The results of the variance component analysis are provided in Table 4.8. A separate analysis was performed for each organism in the panel. The estimated Time to Fail in minutes, overall mean and SD and CV for each of the estimated variance components are provided. Any variance component estimates that were negative were set to zero. The Total variance component was calculated as the square root of the sum of the squared individual components. Confounded sources of variability are shown in the column headings (e.g. Site Instrument and Operator).

TABLE 4.8
Variance component analysis

| | | Variance Components SD(CV %) | | | | | | | | | |
|---|---|---|---|---|---|---|---|---|---|---|---|
| | Mean | Total | | Site/Instr/Oper | | Lot | | Day/LRAP unit | | Reps | |
| Bacillus cerius | 16.6 | 0.84 | (5.1) | 0.22 | (1.3) | 0.56 | (3.4) | 0 | (0) | 0.58 | (3.5) |
| Clostridium perfringens | 16.7 | 0.93 | (5.6) | 0.5 | (3.0) | 0 | (0) | 0.21 | (1.3) | 0.76 | (4.5) |
| Escherichia coli | 16.3 | 0.84 | (5.1) | 0 | (0) | 0.48 | (3.0) | 0.42 | (2.6) | 0.53 | (3.3) |
| Klebsiella oxytoca | 18.0 | 2.13 | (11.8) | 0 | (0) | 1.28 | (7.1) | 0.93 | (5.2) | 1.43 | (8.0) |
| Propionibacterium acnes | 16.8 | 1.81 | (10.8) | 0 | (0) | 0.8 | (4.7) | 1.13 | (5.7) | 1.17 | (7.0) |
| Pseudomonas aeruginosa | 18.5 | 2.35 | (12.7) | 0 | (0) | 0 | (0) | 1.06 | (5.7) | 2.1 | (11.4) |
| Serratia marcescens | 18.6 | 1.62 | (8.7) | 0.59 | (3.2) | 0.27 | (1.5) | 0 | (0) | 1.48 | (8.0) |
| Staphylococcus aureus | 17.9 | 1.27 | (7.1) | 0.19 | (1.0) | 0.54 | (3) | 0.49 | (2.7) | 1.03 | (5.7) |
| Staphylococcus epidermis | 18.0 | 1.32 | (7.3) | 0.29 | (1.6) | 0.59 | (3.3) | 0.45 | (2.5) | 1.06 | (5.9) |
| Streptococcus agalactiae | 17.7 | 1.84 | (10.4) | 0 | (0) | 1.05 | (5.9) | 1.15 | (6.5) | 0.98 | (5.5) |

### Conclusion:

The inter-assay reproducibility of negative samples showed 100% reproducibility of BacTx® Assay testing of 10 replicates of 21 unique, sterile LRAP units using three BacTx® Kit lots The 2-sided 95% confidence intervals are 98.2%-1 00%, and the lower one-sided 95% confidence limit is 98.7%. In the Inter-lot and Inter-assay reproducibility study of spiked samples, 396 BacTx® Assays were performed using 3 kit lots at 3 test sites. All 396 BacTx® Assays yielded the expected result, a concordance of 100% between the actual and expected results. No statistically significant difference in reproducibility was observed between the three sites or between the three lots (p=1.0, Fisher-Freeman-Halton test). Inter-site and inter-lot reproducibility of negative samples was determined during the Specificity Study. 505 sterile, unique LRAP units were tested with a total of 6 kit lots, of which 501 were not positive for the presence of bacteria in the BacTx® Assay. This corresponds to an overall reproducibility of 99.2%, with 2-sided 95% confidence intervals of 98.0%-99.7%. The lower one-sided 95% confidence limit is 98.2%. The replicate precision for Time to Fail across the different organisms is generally below 10% CV, ranging from CVs of 3.3% to 11.4%. To put the Total SD variability into context of the assay, consider that by taking the largest mean, 18.6, and SO, 2.4, from across the 10 organisms, the assay duration of 30 minutes is still more than 4 standard deviations away. This indicates that the assay variation due to these sources is well controlled and unlikely to generate a false pass result.

### Potentially Interfering Substances Testing:

### Overview:

The Interfering Substances study was performed as in Example 3 for Leukocyte Reduced Apheresis Platelets. In the assay detection system, a dedicated photometer is used to monitor the change in absorbance of green colored light that passes through the BacTx® Reaction Tube during the 30 minute assay. Since the determination of a “Fail” or “Pass” result in the BacTx® Assay is based on whether the change in absorbance exceeds 0.5 during the assay, endogenous substances or specific platelet conditions that contribute to sample turbidity may potentially interfere with the BacTx® Assay. These include hyperproteinemia, hypergammaglobulinemia, hemolysis, hypercholesterolemia and lipemia. In addition, specific platelet conditions may also interfere with the proper functioning of the Lysis, Extraction, or Neutralization Reagents used during sample preparation. These conditions include high and low pH, platelet concentration, and red blood cell concentration. The concentrations of interfering substance were tested at pathological levels compared to normal (or reference) levels.

### Methods:

To test each of the substances or conditions described above, 100 positive samples (1 0 samples for each of the 10 bacterial strains, in which the concentration of bacteria was 0.5-1.5 logs above the limit of detection (LOD) determined during the analytical sensitivity study for each strain) and 10 negative samples were prepared using LRAP containing the interfering substance or condition. All of the LRAP units used for this study were 5 days old or less. The concentrations of the ten bacterial strains used for interference testing are listed in Table 4.6. Three lots of BacTx® Bacterial Detection Kits were used to prepare and test 10 samples for each bacterial strain listed in Table 4.6 and 10 negative samples. Of these 10 samples, 3 samples were prepared with one lot, another 3 samples with a second lot, and the remaining 4 samples with a third lot. Specific details for preparing the samples for each condition are described below. A summary of the sample conditions tested can be found in Table 4.9. The concentrations of interfering substan-ceswere tested at pathological levels compared to normal (or reference) levels.

TABLE 4.9
Summary of Inetrfering Substance Conditions and
Normal (Reference) Values

| | | Normal (Reference) |
|---|---|---|
| Condition | Test Concentration | Values |
| Low Platelet Concentration | 50% of normal^{a} | 3.0 × 10¹¹ platelets/ |
| | (5.0-6.0) × 10⁸ | 250-300 mL Unit^{a} |
| | platelets/mL | |
| High Platelet Concentration | 200% of normal^{a} | |
| | (2.0-2.4) × 10⁸ | |
| | platelets/mL | |
| Low pH | 5.5 | 6.2^{b} |
| Normal pH | 7.16-7.21 | 7.38-7.42^{c} |
| High pH | 8.5 | |
| Red Blood Cells | 0.7% hematocrit | 0-0.4%^{d} |
| Hemolysis | 0.25 g/dL hemoglobin | 0-0.13 g/dL^{e} |
| Hyperproteinemia | 10.4 g/dL | 6.0-8.3 g/dL^{f} |
| Hypoproteinemia | 3.2 g/dL | |
| Lipemia | 1350 mg/dL | <150 mg/dL^{g} |
| Hypercholesterolemia | 234 mg/dL | <200 mg/dL^{h} |
| Hypergammaglobulinemia | 3500 mg/dL | 560-1800 mg/dLⁱ |
| (IgG) | | |
| Hypergammaglobulinemia | 1000 mg/dL | 45-250 mg/dLⁱ |
| (IgM) | | |
| Hypergammaglobulinemia | 2205 mg/dL | 100-400 mg/dLⁱ |
| (IgA) | | |

^{a}December 2007. “Guidance for Industry and FDA Review Staff: Collection of Platelets by Automated Methods” Rockville, MD: City, State: U.S. Department of Health and Human Services, Food and Drug Administration, Center for Biologics Evaluation and Research. (Downloaded on Jun. 22, 2011).
^{b}21 CFR, Chap 1, Subpart C, section 640.24 (4-1-05 Edition).
^{c}Normal range of blood serum from “Merck Manual of Diagnosis and Therapy, Section: Endocrine and Metabolic Disorders, Subject: Acid Base Regulation and Disorders. Topic: Introduction” http://www.merckmanuals.com/. James L. Lewis, III, MD (ed). July 2008. Merck Sharp & Dohme Corp. Accessed Jun. 22, 2011 <http://www.merckmanuals.com/professional/sec12/ch157/ch157b.html>.
^{d}Upper limit based on 2 mL of packed red blood cells in 500 mL plasma volume, as described in (b).
^{e}Upper limit based on complete hemolysis of 0.4% hematocrit.
^{f}Based on normal serum total protein level from: David C. Dugdale. “Total Protein”. http://www.nlm.nih.gov/medlineplus/medlineplus.html. David Zieve (Rev). May 2009. U.S. National Library of Medicine, U.S. Department of Health and Human Services, National Institutes of Health. Accessed Jun. 22, 2011 <http://www.nlm.nih.gov/medlineplus/ency/article/003483.htm>.
^{g}Based on serum triglyceride reference range classified as desirable from “Triglycerides”. http://www.labtestsonline.org. March 2011. American Association for Clinical Chemistry. Accessed Jun. 22, 2011 <http://www.labtestsonline.org/understanding/analytes/triglycerides/sample.html>.
^{h}Based on serum cholesterol reference range classified as desirable from “Cholesterol”. http://www.labtestsonline.org. March 2011. American Association for Clinical Chemistry. Accessed Jun. 22, 2011 <http://www.labtestsonline.org/understanding/analytes/cholesterol/test.html>.
ⁱBased on normal serum levels from: David C. Dugdale. “Quantitative Nephelometry”. http://www.nlm.nih.gov/medlineplus/medlineplus.html. David Zieve (Rev). June 2010. U.S. National Library of Medicine, U.S. Department of Health and Human Services, National Institutes of Health. Accessed Jun. 22, 2011 <http://www.nlm.nih.gov/medlineplus/ency/article/003545.htm>.

### Platelet Concentration:

To simulate an LRAP unit with an abnormally high platelet concentration, an LRAP unit was split into two volumes and one volume was centrifuged at low speed in a clinical centrifuge. The plasma was decanted and the platelets were gently resuspended in the second volume of platelets. The resulting sample had twice the normal concentration of platelets. To simulate a platelet pool with an abnormally low platelet concentration, an LRAP unit was split into two volumes and one volume was centrifuged at low speed in a clinical centrifuge. The plasma was transferred to the second volume of platelets. The resulting sample had half the normal concentration of platelets.

### High, Low, and Normal pH:

To simulate a LRAP unit with abnormally high or low pH, 1 N NaOH or 1 N HCl, respectively, was added to LRAP and the pH was measured using a pH meter. The low pH range tested was 5.5, which is below the pH threshold (6.2) used for quality control of manufactured platelets at time of issue or expiry. The upper pH range tested was 8.5, which is almost a full pH unit above the normal pH range of serum. For LRAP at normal pH, the pH was measured using a calibrated pH meter and found to be 7.16 and 7.21 on two different days.

### Red Blood Cell Contamination (Hematocrit):

To simulate LRAP with abnormally high amounts of red blood cells, a volume of packed RBCs (70.5% hematocrit) was added to LRAP to yield a final hematocrit of 0.7%. The hematocrit of the packed RBCs was approximated by multiplying the measured hemoglobin concentration (in g/dL) in the packed RBCs by three. The hemoglobin concentration was measured using a commercially available kit (based on Drabkin's reagent). A hematocrit of 0.7% is almost twice the allowable level for manufactured platelets (0.4%).

### Hemolysis:

To simulate a hemolytic LRAP, hemolyzed red blood cells were first produced by diluting several milliliters of packed red blood cells (RBCs) with an equal volume of sterile PBS. The diluted RBCs were lysed by intermittent sonication using a probe sonicator. The RBC lysate was separated from the intact RBCs by centrifugation at 2000×g for 10 minutes. The hemoglobin content of the supernatant (RBC lysate) was measured using a commercially available kit (based on Drabkin's reagent). A volume of lysed RBCs was added to LRAP to yield a final hemoglobin concentration of 0.25 g/dL, almost twice the acceptable upper limit (0.13 g/dL) for manufactured platelets.

Hyoerproteinemia/Hypergammaglobulinemia (lgG):

To simulate a hyperproteinemic and hypergammaglobulinemic LRAP, LRAP was centrifuged at low speed in a clinical centrifuge. The plasma was decanted and replaced with an equal volume of hyperproteinemic plasma, consisting of purified human gammaglobulins (Sigma-Aldrich) dissolved in human plasma (isolated from LRAP) at a concentration of 40 mg/mL. The platelets were gently resuspended in the hyperproteinemic plasma. The protein concentration of the hyperproteinemic LRAP was determined by Biuret assay and found to be 10.4 g/dL, which is above the normal range for serum (6.0-8.3 g/dL). The lgG concentration in the platelets was determined by commercially available human lgG ELISA kit and found to be 3500 mg/dL, which is almost twice the upper limit observed for normal serum level (1800 mg/dL) for adults.

### Hypoproteinemia:

To simulate a hypoproteinemic LRAP, LRAP was centrifuged at low speed and half of the plasma was replaced with sterile PBS. The platelets were gently resuspended in the diluted plasma with a serological pipet. The protein concentration of the resulting hypoproteinemic platelet pool was measured by Biuret assay and found to be 3.2 g/dL, which is approximately half the lower limit that is considered normal (6.0 g/dL).

### Lipemia:

To simulate a lipemic LRAP, a volume of LRAP was centrifuged at low speed to separate the platelets and plasma. After decanting the plasma fraction, the platelets were gently resuspended in the same volume of lipemic plasma from a single donor. The triglycerides concentration in the lipemic LRAP was determined using a commercially available kit and found to be 1350 mg/dL, which is nine times greater than the desirable reference level of 150 mg/dl for adults.

### Hypercholesterolemia:

To prepare a hypercholesterolemic LRAP, a volume of LRAP was centrifuged at low speed in a clinical centrifuge. The plasma was decanted and the platelets were gently resuspended in an equal volume of hypercholesterolemic serum, pooled from 115 individual donors. The cholesterol concentration in the hypercholesterolemic LRAP was 234 mg/dL, which is considered high.

Hypergammaglobulinemia (lgM):

To simulate hypergammaglobulinemic platelet pool with abnormally high levels of lgM, a volume of LRAP was centrifuged at low speed. 20% of the plasma supernatant volume was replaced with hypergammaglobulinemia plasma (commercially available lgM-positive myeloma patient plasma at 5000 mg/dL) and the platelets were gently resuspended using a serological pipet, to yield LRAP with lgM level of 1000 mg/dL, which is four times the normal serum level (250 mg/dL) for adults.

Hypergammaglobulinemia (lgA):

To simulate hypergammaglobulinemic platelet pool with abnormally high levels of lgA, a volume of LRAP was centrifuged at low speed. A volume of the plasma supernatant was replaced with hypergammaglobulinemia plasma (commercially available lgA-positive myeloma patient plasma at 2205 mg/dl) and the platelets were gently resuspended using a serological pipet. to yield LRAP with lgA level of 2205 mg/dL. which is more than five times the normal serum level (400 mg/dl) for adults. Results of Potentially Interfering Substances Testing

### Discussion:

The BacTx® Assay results for the interfering substances study are shown in Table 4.10. Out of the 1300 positive samples tested, 100% were detected with the BacTx® Assay. Out of the 130 negative samples tested, no false positives were observed. Based on these results, the following substances and platelet conditions do not interfere with the BacTx® Assay: 50-200% normal platelet concentration, low and high pH, 0.7% hematocrit, hemolysis, hyperproteinemia, hypoproteinemia, lipemia, hypercholesterolemia, and hypergammaglobinemia (lgA, lgG, and lgM).

### Conclusion:

No conditions were tested that generated false results in the BacTx® Assay. One hundred percent of 1300 positive BacTx® Assays gave the expected result, as did 100% of 130 negative samples tested. Based on these results, the following substances and platelet conditions do not interfere with the BacTx® Assay: 50-200% normal platelet concentration, low and high pH, 0.7% hematocrit, hemolysis, hyperproteinemia, hypoproteinemia, lipemia, hypercholesterolemia, and hypergammaglobinemia (lgA, lgG, and lgM).

TABLE 4.10
Summary of Interference Testing Results

| | # BacTx ® Assay Results Yielding the Expected Results | | | | | |
|---|---|---|---|---|---|---|
| | 50% Patient | 200% Patient | Low | Normal | High | Hematocrit |
| Bacteria | Concentration | Concentration | pH | pH | pH | (0.7%) |
| Escherichia coli | 10/10 | 10/10 | 10/10 | 10/10 | 10/10 | 10/10 |
| Staphylococcus aureus | 10/10 | 10/10 | 10/10 | 10/10 | 10/10 | 10/10 |
| Bacillus cerius | 10/10 | 10/10 | 10/10 | 10/10 | 10/10 | 10/10 |
| Staphylococcus epidermis | 10/10 | 10/10 | 10/10 | 10/10 | 10/10 | 10/10 |
| Klebsiella oxytoca | 10/10 | 10/10 | 10/10 | 10/10 | 10/10 | 10/10 |
| Pseudomonas aeruginosa | 10/10 | 10/10 | 10/10 | 10/10 | 10/10 | 10/10 |
| Streptococcus agalactiae | 10/10 | 10/10 | 10/10 | 10/10 | 10/10 | 10/10 |
| Serratia marcescens | 10/10 | 10/10 | 10/10 | 10/10 | 10/10 | 10/10 |
| Clostridium perfringens | 10/10 | 10/10 | 10/10 | 10/10 | 10/10 | 10/10 |
| Propionibacterium acnes | 10/10 | 10/10 | 10/10 | 10/10 | 10/10 | 10/10 |
| Negative Assays | 10/10 | 10/10 | 10/10 | 10/10 | 10/10 | 10/10 |

| | # BacTx ® Assay Results Yielding the Expected Results | | | | | | |
|---|---|---|---|---|---|---|---|
| | | Hypo- | | Hyper- | Hypoproteinemia/ | | |
| Bacteria | Hemolysis | proteinemia | Lipemia | cholesterol | Hyper-IgG | Hyper-IgA | Hyper-IgM |
| Escherichia coli | 10/10 | 10/10 | 10/10 | 10/10 | 10/10 | 10/10 | 10/10 |
| Staphylococcus aureus | 10/10 | 10/10 | 10/10 | 10/10 | 10/10 | 10/10 | 10/10 |
| Bacillus cerius | 10/10 | 10/10 | 10/10 | 10/10 | 10/10 | 10/10 | 10/10 |
| Staphylococcus epidermis | 10/10 | 10/10 | 10/10 | 10/10 | 10/10 | 10/10 | 10/10 |
| Klebsiella oxytoca | 10/10 | 10/10 | 10/10 | 10/10 | 10/10 | 10/10 | 10/10 |
| Pseudomonas aeruginosa | 10/10 | 10/10 | 10/10 | 10/10 | 10/10 | 10/10 | 10/10 |
| Streptococcus agalactiae | 10/10 | 10/10 | 10/10 | 10/10 | 10/10 | 10/10 | 10/10 |
| Serratia marcescens | 10/10 | 10/10 | 10/10 | 10/10 | 10/10 | 10/10 | 10/10 |
| Clostridium perfringens | 10/10 | 10/10 | 10/10 | 10/10 | 10/10 | 10/10 | 10/10 |
| Propionibacterium acnes | 10/10 | 10/10 | 10/10 | 10/10 | 10/10 | 10/10 | 10/10 |
| Negative Assays | 10/10 | 10/10 | 10/10 | 10/10 | 10/10 | 10/10 | 10/10 |

### EQUIVALENTS

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

1-85. (canceled)

86. A method of analyzing a sample, said method comprising:
a) processing the sample;
b) adding the sample to a reaction tube;
c) scanning a barcode;
d) inserting the reaction tube to the reaction well of an assay device;
wherein said device comprises:
i. an optical reader apparatus, said apparatus comprises:
1) a light source;
2) at least one lens in optical alignment with light reflected, transmitted through, or emitted from the sample;
3) a detector for capturing the reflected light from the sample, light transmitted through the sample, or emitted from the sample;
4) a sample mixing subsystem comprising at least one reaction well;
5) an onboard computer;
6) a touchscreen monitor comprising a graphical user interface;
ii. a processor running software, said software comprises an algorithm for:
1) calculating an absorbance of the sample;
2) matching a barcode to sample; and
iii. a barcode scanner
e) initiating a light source on the sample in the reaction tube;
f) detecting the light reflected from the sample, emitted or transmitted through the sample;
g) processing and analyzing the data from step f);
h) generating a test result.

87. The method according to claim 86, wherein the absorbance are quantitatively assessed.

88. The method according to claim 86, further comprising the step of affixing a BacTx ID manufacturer's barcode to the reaction tube.

89. (canceled)

90. The method according to claim 86, further comprising the step of mixing the reaction tube prior to step e).

91. The method according to claim 86, further comprising the step of standardizing the test data or results.

92. The method according to claim 86, further comprising a calibration step prior to step a).

93. (canceled)

94. The method according to claim 86, wherein the test result is stored internally in the assay device or transmitted to an external device, electronically, via a handheld device, or wirelessly.

95. (canceled)

96. (canceled)

97. (canceled)

98. (canceled)

99. The method according to claim 86, further comprising the step of matching the ISBT sample ID to a BacTx ID.

100. (canceled)

101. The method according to claim 86, wherein said test result of “PASS” indicates compliance of said sample and a test result of “FAIL” indicates lack of compliance of said sample.

102. (canceled)

103. The method according to claim 86, where the test results are matched to barcodes identifying sample ID, BacTx ID, test run, and test date.

104. The method according to claim 86, further comprising the step of notifying the user, medical practitioner, third party, or laboratory manager of the test results.

105. The method according to claim 104, further comprising the step of using the test data in diagnosing, prognosing, or treating of urinary tract infections, bacterial meningitis, bacterial infections of the CNS, bacterial infections, viral infections, and fungal infections.

106. The method according to claim 105, wherein the diagnosis, prognosis, or treatment is based on the patient's own previous pharmacodynamic, pharmacokinetic, or pharmacogenetic profiles.

107. The method according to claim 106, further comprising using the test data to monitor patient diagnosis, prognosis or therapy.

108. The method according to claim 86, further comprising the step of comparing the absorbance present in the sample and determining the absence or presence of bacteria, fungi, viruses, or contaminants in the sample.

109. The method according to claim 86, further comprising the step of cross-referencing the test result to medical records of a patient with the at least one pharmacological parameter to assistant a clinician in providing an individualized medical treatment.

110. The method according to claim 106, further comprising the step of correlating the absorbance to establish compliance or non-compliance thresholds.

111. The method according to claim 86, wherein the test results provide biometric identification of a compliant or noncompliant blood sample to a provider or user of the sample.

112. The method according to claim 86, further comprising a pre-processing step before step a).

113. The method according to claim 86, wherein the processing step a) comprises: incubating the sample with a prophenoloxidase cascade system, a phenoloxidase substrate that generates a quinone reaction product, and 3-methyl-2-benzothiazolinone hydrazone; and, detecting the formation of a colored phenoloxidase reaction product, wherein formation of the reaction product indicates the presence of a bacterial, fungal, or viral contaminant in the sample.

114. The method according to claim 86, wherein the processing step a) comprises: (a) extracting the sample in an alkaline extraction solution, (b) incubating the sample with silkworm larvae plasma, L-3,4-dihydroxyphenolalanine, and 3-methyl-2-benzothiazolinone hydrazone, wherein the 3-methyl-2-benzothiazolinone hydrazone is dissolved in neutralization buffer, and (c) detecting the formation of a colored prophenoloxidase reaction product.

115. The method according to claim 113, wherein formation of the reaction product indicates the presence of fungi in the sample.

116. The method according to claim 113, wherein the sample is a clinical sample, an environmental sample, an agricultural sample, a manufacturing sample, or a medical product.

117. The method according to claim 116, wherein the clinical sample is a hydration fluid, nutrient fluid, blood, blood product, vaccine, anesthetic, pharmacologically active agent, platelets, or an imaging agent.

118. (canceled)

119. (canceled)

120. (canceled)

121. The method according to claim 116, wherein the sample is a suspension or a liquid and processed by centrifugation wherein bacteria or fungi present in the sample are pelleted during centrifugation.

122. The method according to claim 113, wherein the prophenoloxidase cascade system comprises prophenoloxidase activating enzyme, prophenoloxidase, and a serine proteinase cascade.

123. The method according to claim 122, wherein the system further comprises a peptidoglycan binding protein, β-glucan binding protein, a peptidoglycan standard, a β-glucan standard, a bacterial standard, or bacterial fragment standard; said peptidoglycan standard is selected an isolated bacterial peptidoglycan, whole bacterial extract, or inactivated whole bacteria.

124. (canceled)

125. The method according to claim 122, wherein the prophenoloxidase cascade system is obtained from insect plasma, silkworm larvae plasma, or hemolymph.

126. (canceled)

127. The method according to claim 113, wherein the phenoloxidase substrate that generates a quinone reaction product is L-3,4-dihydroxyphenolalanine dopamine, 3,4-dihydroxyphenyl propionic acid, 3,4-dihydroxyphenyl acetic acid, a dihydroxyphenol, a monophenol, or catechol.

128. The method according to claim 86, further comprising the step of exposing the sample to an alkaline extraction solution, prior to incubating the sample with the prophenoloxidase cascade system, the phenoloxidase substrate that generates a quinone reaction product, and 3-methyl-2-benzothiazolinone hydrazone.

129. (canceled)

130. The method according to claim 86, further comprising the step of exposing the sample to a neutralization buffer prior to incubating the sample with the prophenoloxidase cascade system, the phenoloxidase substrate that generates a quinone reaction product, and 3-methyl-2-benzothiazolinone hydrazone.

131. The method according to claim 86, further comprising the step of exposing the sample to a neutralization buffer containing 3-methyl-2-benzothizolinone dissolved therein prior to incubating the sample with the prophenoloxidase cascade system, the phenoloxidase substrate that generates a quinone reaction product, and 3-methyl-2-benzothiazolinone hydrazone.

132-135. (canceled)

136. The method according to claim 86, which is adapted to run a colorimetric assay, and further comprising instructions for spectrophotometric detection or a color coded scale for visual evaluation.

137. The method according to claim 86, wherein the assay reader further comprises a sterile or aseptic sample receptacle.
